(19) United States
(12) Patent Application Publication
(10) Pub. No.: US 2026/0077088 A1
(43) Pub. Date: Mar. 19, 2026

(54) SELF-DEGRADING ENZYME LOADED BIOLOGICALLY DERIVED PARTICLES

(71) Applicant: CRANNMED LIMITED, Galway (IE)

(72) Inventors: Sankalp AGARWAL, Co. Galway (IE); Masum PANDEY, Co. Galway (IE); Andrew LEWIS, Galway (IE)

(73) Assignee: CRANNMED LIMITED, Galway (IE)

(21) Appl. No.: 19/109,241

(22) PCT Filed: Sep. 1, 2023
(86) PCT No.: PCT/IB2023/058680

§ 371 Date: Mar. 6, 2025

Related U.S. Application Data
(60) Provisional application No. 63/374,833, filed on Sep. 7, 2022.

(51) Int. Cl. A61L 24/08; A61L 24/00; C12N 9/88

(52) U.S. Cl. CPC A61L 24/08; A61L 24/0015; A61L 24/0042; C12N 9/88; A61L 2300/254; A61L 2300/622; A61L 2400/06; A61L 2430/36; C12Y 402/02003; C12Y 402/02009

(57) ABSTRACT

The present disclosure provides compositions including enzyme loaded biologically derived microspheres capable of self-degradation upon rehydration. The present disclosure also provides methods of making the enzyme loaded biologically derived microspheres. The present disclosure also provides methods of using the disclosed microspheres to induce an embolism in a subject as well as methods of using the disclosed microspheres to treat a disease or disorder in a subject.

### CROSS-REFERENCE TO RELATED APPLICATIONS

The present application claims priority to U.S. Provisional Application No. 63/374,833, filed Sep. 7, 2022, which is incorporated herein by reference in its entirety.

### FIELD

The present disclosure provides compositions comprising self-degrading enzyme loaded biologically derived particles. The disclosure further provides methods of making the enzyme loaded biologically derived particles, including methods of crosslinking the particles using divalent ions and/or photo-crosslinking. In some aspects, post-preparation processing (e.g., sterilization and dehydration) of the enzyme loaded biologically derived particles is used for improving shelf-life. The disclosure further provides methods of using the enzyme loaded biologically derived particles to treat a disease or disorder in a subject that would benefit from the administration of these self-degrading particles. The disclosure further provides methods of using the enzyme loaded biologically derived particles in an embolization procedure in a subject in need thereof.

### BACKGROUND

Artificial blocking of a blood vessel, or embolization, in an organ may be used, for example, (a) to control bleeding caused due to trauma, (b) to prevent blood flow into abnormal blood vessels such as aneurysms, and/or (c) to treat an organ (e.g., to excise a tumor, for transplant, or for surgery). In many circumstances, the permanent embolization of blood vessels is not required. For such medical interventions, using temporary and bioresorbable embolic agents is desirable. For example, IMP/CS (Imipenem/Cilastatin) antibiotic particles of size ranging from 10 μm to 80 μm have been used as a temporary embolic agent, however this material can require nearly a month to become absorbed completely (see, e.g., Okuno, et al.; “Midterm Clinical Outcomes and MR Imaging Changes after Transcatheter Arterial Embolization as a Treatment for Mild to Moderate Radiographic Knee Osteoarthritis Resistant to Conservative Treatment”, J. Vasc. Interv. Radiol. 2017; 28:995-1002). Similarly, other embolic agents such as Gelfoam®, collagen, and thrombin have also been used (see, e.g., Vaidya, et al.; “An overview of embolic agents”, Semin. Intervent. Radiol. 2008; 25:204-15). However, existing agents have numerous drawbacks such as unpredictable resorption rate, lack of agent(s) that selectively degrade abovementioned matrices, and/or migration of the embolic agents causing non-specific occlusion (see, e.g., U.S. Patent Application Publication No. 20130211249). Furthermore, some embolic agents require a processing or preparation step before their use within the body. For example, Gelfoam has to be cut up into pledgets or slurried. Likewise, autologous blood clots have to be collected formed and re-injected.

Temporary, self-degrading agents also have uses outside of embolization and may be used to treat various diseases or disorders in a subject in need thereof. For example, such agents may be used in tissue bulking applications, such as cosmetic fillers and sphincter bulking materials, to provide temporary mechanical support during bone healing, and organ-spacing materials (such as SpaceOAR™ which is used as a temporary spacer between prostate and bowel to protect the bowel during radiotherapy for prostate cancer treatment). More generally, self-degrading agents may be used for degradable implant materials for drug delivery.

Accordingly, there is a need for self-degrading agents that can exhibit a predictable dissolution rate as well as methods of making these self-degrading agents. Additionally, there is a need in the art for methods of using these self-degrading agents to treat a disease or disorder in a subject or in an embolization procedure in a subject without creating any non-specific occlusion in vivo.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY OF THE DISCLOSURE

In one aspect, the present disclosure provides a microsphere capable of self-degradation upon rehydration, comprising: an enzyme pre-treated by varying temperature, by varying pH, and/or with a metal-ion enzyme inhibitor; and a crosslinked biomaterial; wherein: the crosslinked biomaterial forms a biologically derived microsphere encapsulating the enzyme; and

the microsphere is substantially free of water and/or sterilized. In one embodiment, at least one of (i)-(iv) applies: (i) the enzyme is an enzyme that acts on the biomaterial, degrading the biomaterial; (ii) the biomaterial comprises a polysaccharide, a protein, or a glycoprotein; (iii) the microsphere further comprises a photoinitiator and the biomaterial comprises a photo-crosslinkable moiety which is photo-crosslinked; or (iv) the biomaterial is crosslinked by a divalent metal ion. In one embodiment, the self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial. In one embodiment, the enzyme activity is between about 0.0075 U/mg to 0.25 U/mg of the biomaterial and the microsphere degrades over a period of greater than about 20 minutes to less than about 4 hours; the enzyme activity is between about 0.005 U/mg to about ≥0.0025 U/mg of the biomaterial and the microsphere degrades over a period of greater than about 5 days to less than about 30 days; or the enzyme activity is less than about 0.0025 U/mg of the biomaterial, and the microsphere degrades over a period of greater than about 30 days. In one embodiment, at least one of (i)-(iv) applies: (i) the residual water content of the microsphere is between about 1% by mass and about 10% by mass; (ii) the microsphere is lyophilized or dehydrated using super critical CO₂; (iii) the microsphere is sterilized with about 6-10 kGy of gamma radiation; or (iv) the microsphere further comprises an anti-inflammatory agent, a chemotherapeutic agent, an antioxidant, a corticosteroid, or a combination thereof. In one embodiment, the biomaterial comprises alginate and the enzyme is alginate lyase, the biomaterial comprises pectin and the enzyme is pectinase, the biomaterial comprises hyaluronic acid and the enzyme is hyaluronidase, the biomaterial comprises gelatin and the enzyme is a matrix metalloproteinase or protease, the biomaterial comprises albumin and the enzyme is peptidase, the biomaterial comprises collagen and the enzyme is protease, the biomaterial comprises fibrinogen and the enzyme is plasmin, the biomaterial comprises silk fibrin and the enzyme is protease, the biomaterial comprises starch and the enzyme is amylase, the biomaterial comprises chitosan and the enzyme is chitosanase or lysozyme, the biomaterial comprises agar/agarose and the enzyme is agarase, the biomaterial comprises carrageenan and the enzyme is carrageenase, the biomaterial comprises pullulan and the enzyme is pullulanase, the biomaterial comprises dextran and the enzyme is dextranase, the biomaterial comprises b-glycan and the enzyme is b-glycanase, the biomaterial comprises cellulose and the enzyme is cellulase, or the biomaterial comprises lignin and the enzyme is ligninase.

In another aspect, the present disclosure provides a method of preparing a microsphere capable of self-degradation upon rehydration, the method comprising: forming droplets from a precursor solution, the precursor solution comprising: (i) an enzyme pre-treated by varying temperature, by varying pH, and/or with a metal-ion enzyme inhibitor; and (ii) a biomaterial;
contacting the droplets with a gelling bath comprising a cryoprotectant and a divalent metal ion, thereby cross-linking the biomaterial to form a biologically derived microsphere encapsulating the enzyme; and dehydrating, and optionally sterilizing, the microsphere thereby substantially removing water from the microsphere. In one embodiment, at least one of (i)-(v) applies: (i) the precursor solution further comprises one or more cryoprotectants; (ii) the enzyme is an enzyme that acts on the biomaterial, degrading the biomaterial; (iii) the biomaterial comprises a polysaccharide, a protein, or a glycoprotein; (iv) the microsphere comprises: alginate particles encapsulating alginate lyase, pectin particles encapsulating pectinase, gelatin particles encapsulating a matrix metalloproteinase or protease, or carrageenan particles encapsulating carrageenase; or (v) the microsphere self-degrades in less than about 4 hours, over a period of greater than about 2 days to less than about 5 days, over a period of greater than about 5 days to less than about 30 days, or greater than about 30 days. In one embodiment, the self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial. In one embodiment, the droplets are contacted with the gelling bath for a range of 10 minutes to 1 hour and the resulting microspheres degrade over a period of greater than about 20 minutes to less than about 4 hours; the droplets are contacted with the gelling bath for a range of 1 hour to 12 hours and the resulting microspheres degrade over a period of greater than about 5 days to less than about 30 days; or the droplets are contacted with the gelling bath for a range of 12 hours to 24 hours and the resulting microspheres degrade over a period of greater than about 30 days. In one embodiment, at least one of (i)-(iv) applies: (i) the residual water content of the microsphere is between about 1% by mass and about 10% by mass; (ii) the dehydrating comprises lyophilizing the microsphere or drying the microsphere using super critical CO₂; (iii) the sterilizing comprises irradiating the microsphere with 6-10 kGy of gamma radiation; or (iv) the precursor solution and/or the gelling bath further comprise an anti-inflammatory agent, a chemotherapeutic agent, an antioxidant, or a combination thereof.

In yet another aspect, the present disclosure provides a method of preparing a photopolymerized microsphere capable of self-degradation upon rehydration, the method comprising: forming droplets from a precursor solution, the precursor solution comprising: (i) an enzyme pre-treated by varying temperature, by varying pH, and/or with a metal-ion enzyme inhibitor; (ii) a biomaterial comprising a photo-crosslinkable moiety; (iii) a photoinitiator; irradiating the droplets, thereby cross-linking the biomaterial to form a photo-polymerized biologically derived microsphere encapsulating the enzyme; and dehydrating, and optionally sterilizing, the microsphere thereby substantially removing water from the microsphere. In one embodiment, at least one of (i)-(v) applies: (i) the photo-crosslinkable moiety is selected from an acrylate group, a methacrylate group, a vinyl group, and an allyl group; (ii) the precursor solution further comprises one or more cryoprotectants; (iii) the enzyme is an enzyme that acts on the biomaterial, degrading the biomaterial; (iv) the biomaterial comprises a polysaccharide, a protein, or a glycoprotein; or (v) the microsphere self-degrades in less than about 4 hours, over a period of greater than about 2 days to less than about 5 days, over a period of greater than about 5 days to less than about 30 days, or greater than about 30 days. In one embodiment, the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase. In one embodiment, the self-degradation of the microsphere is controlled by one or more of: the pre-treatment of the enzyme, the concentration of the enzyme in the microsphere, the enzyme activity, the predetermined molecular weight of the biomaterial, and the amount of time that the droplets are irradiated. In one embodiment, at least one of (i)-(iv) applies: (i) the residual water content of the microsphere is between about 1% by mass and about 10% by mass; (ii) the dehydrating comprises lyophilizing the microsphere or drying the microsphere using super critical CO₂; (iii) the sterilizing comprises irradiating the microsphere with 6-10 kGy of gamma radiation; or (iv) the precursor solution further comprises an anti-inflammatory agent, a chemotherapeutic agent, an antioxidant, or a combination thereof.

In yet another aspect, the present disclosure provides a method of preparing a microsphere capable of self-degradation upon rehydration, the method comprising: forming droplets from a precursor solution, the precursor solution comprising: (i) a biomaterial comprising a covalently crosslinkable moiety; and (ii) a homo-bifunctional crosslinking agent or a heterobifunctional crosslinking agent; covalently cross-linking the biomaterial to form a biologically derived microsphere; swelling an enzyme that has been pre-treated by varying temperature, by varying pH, and/or with a metal-ion enzyme inhibitor into the microsphere such that the biologically derived microsphere encapsulates the enzyme; and dehydrating, and optionally sterilizing, the microsphere thereby substantially removing water from the microsphere. In one embodiment, at least one of (i)-(v) applies: (i) the covalently crosslinkable moiety comprises an amine group or a carboxyl group; (ii) the precursor solution further comprises one or more cryoprotectants; (iii) the enzyme is an enzyme that acts on the biomaterial, degrading the biomaterial; (iv) the biomaterial comprises a polysaccharide, a protein, or a glycoprotein; or (v) the microsphere self-degrades in less than about 4 hours, over a period of greater than about 2 days to less than about 5 days, over a period of greater than about 5 days to less than about 30 days, or greater than about 30 days. In one embodiment, the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase. In one embodiment, the self-degradation of the microsphere is controlled by one or more of: the pre-treatment of the enzyme, the concentration of the enzyme in the microsphere, the enzyme activity, the predetermined molecular weight of the biomaterial, and the homo-bifunctional crosslinking agent or heterobifunctional crosslinking used to crosslink the biomaterial. In one embodiment, at least one of (i)-(iv) applies: (i) the residual water content of the microsphere is between about 1% by mass and about 10% by mass; (ii) the dehydrating comprises lyophilizing the microsphere or drying the microsphere using super critical CO₂; (iii) the sterilizing comprises irradiating the microsphere with 6-10 kGy of gamma radiation; or (iv) the precursor solution further comprises an anti-inflammatory agent, a chemotherapeutic agent, an antioxidant, or a combination thereof.

In yet another aspect, the present disclosure provides a method of preparing a microsphere capable of self-degradation upon rehydration, the method comprising: forming droplets from a precursor solution, the precursor solution comprising: (i) a biomaterial comprising a covalently crosslinkable moiety; (ii) an enzyme that has been pre-treated by varying temperature, by varying pH, and/or with a metal-ion enzyme inhibitor; and (iii) a homo-bifunctional crosslinking agent or a heterobifunctional crosslinking agent; covalently cross-linking the biomaterial to form a biologically derived microsphere encapsulating the enzyme; and dehydrating, and optionally sterilizing, the microsphere thereby substantially removing water from the microsphere. In one embodiment, at least one of (i)-(v) applies: (i) the covalently crosslinkable moiety comprises an amine group or a carboxyl group; (ii) the precursor solution further comprises one or more cryoprotectants; (iii) the enzyme is an enzyme that acts on the biomaterial, degrading the biomaterial; (iv) the biomaterial comprises a polysaccharide, a protein, or a glycoprotein; or (v) the microsphere self-degrades in less than about 4 hours, over a period of greater than about 2 days to less than about 5 days, over a period of greater than about 5 days to less than about 30 days, or greater than about 30 days. In one embodiment, the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase. In one embodiment, the self-degradation of the microsphere is controlled by one or more of: the pre-treatment of the enzyme, the concentration of the enzyme in the microsphere, the enzyme activity, the predetermined molecular weight of the biomaterial, and the homo-bifunctional crosslinking agent or heterobifunctional crosslinking used to crosslink the biomaterial. In one embodiment, at least one of (i)-(iv) applies: (i) the residual water content of the microsphere is between about 1% by mass and about 10% by mass; (ii) the dehydrating comprises lyophilizing the microsphere or drying the microsphere using super critical CO₂; (iii) the sterilizing comprises irradiating the microsphere with 6-10 kGy of gamma radiation; or (iv) the precursor solution further comprises an anti-inflammatory agent, a chemotherapeutic agent, an antioxidant, or a combination thereof.

In yet another aspect, the present disclosure provides a method of preparing a thermogelated microsphere capable of self-degradation upon rehydration, the method comprising: heating a precursor solution comprising a biomaterial to melt the biomaterial; adding to the precursor solution an enzyme pre-treated by varying temperature, by varying pH, and/or with a metal-ion enzyme inhibitor; forming droplets from the precursor solution; cooling the droplets to form a thermogelated biologically derived microsphere encapsulating the enzyme; and dehydrating, and optionally sterilizing, the microsphere thereby substantially removing water from the microsphere. In one embodiment, at least one of (i)-(iv) applies: (i) the precursor solution further comprises one or more cryoprotectants; (ii) the enzyme is an enzyme that acts on the biomaterial, degrading the biomaterial; (iii) the biomaterial comprises a polysaccharide, a protein, or a glycoprotein; or (iv) the microsphere self-degrades in less than about 4 hours, over a period of greater than about 2 days to less than about 5 days, over a period of greater than about 5 days to less than about 30 days, or greater than about 30 days. In one embodiment, the microsphere comprises pectin particles encapsulating pectinase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, or agar/agarose particles encapsulating agarose. In one embodiment, the self-degradation of the microsphere is controlled by one or more of: the pre-treatment of the enzyme, the concentration of the enzyme in the microsphere, the enzyme activity, and the predetermined molecular weight of the biomaterial. In one embodiment, at least one of (i)-(iv) applies: (i) the residual water content of the microsphere is between about 1% by mass and about 10% by mass; (ii) the dehydrating comprises lyophilizing the microsphere or drying the microsphere using super critical CO₂; (iii) the sterilizing comprises irradiating the microsphere with 6-10 kGy of gamma radiation; or (iv) the precursor solution further comprises an anti-inflammatory agent, a chemotherapeutic agent, an antioxidant, or a combination thereof.

In yet another aspect, the present disclosure provides a method of preparing a microsphere capable of self-degradation, the method comprising: forming a precursor solution, the precursor solution comprising: (i) an enzyme; and (ii) a biomaterial; passing the precursor solution through a needle under the influence of an electrostatic potential, forming droplets; and contacting the droplets with a gelling bath comprising a divalent metal ion, thereby cross-linking the biomaterial to form a biologically derived microsphere encapsulating the enzyme. In one embodiment, the method further comprises dehydrating, and optionally sterilizing, the microsphere thereby substantially removing water from the microsphere to form a microsphere capable of self-degradation upon rehydration. In one embodiment, at least one of (i)-(v) applies: (i) the precursor solution further comprises one or more cryoprotectants; (ii) the enzyme is an enzyme that acts on the biomaterial, degrading the biomaterial; (iii) the biomaterial comprises a polysaccharide, a protein, or a glycoprotein; (iv) the microsphere self-degrades in less than about 4 hours, over a period of greater than about 2 days to less than about 5 days, over a period of greater than about 5 days to less than about 30 days, or greater than about 30 days; or (v) the self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial. In one embodiment, the microsphere comprises: alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase. In one embodiment, at least one of (i)-(iv) applies: (i) the residual water content of the microsphere is between about 1% by mass and about 10% by mass; (ii) the dehydrating comprises lyophilizing the microsphere or drying the microsphere using super critical CO₂; (iii) the sterilizing comprises irradiating the microsphere with 6-10 kGy of gamma radiation; or (iv) the precursor solution and/or the gelling bath further comprise an anti-inflammatory agent, a chemotherapeutic agent, an antioxidant, or a combination thereof.

In yet another aspect, the present disclosure provides a method of inducing a self-degrading embolism in a subject in need thereof, comprising administering a plurality of the microspheres described above into a blood vessel of the subject. In one embodiment, the blood vessel is a geniculate artery and/or the method induces a prostate arterial embolism, induces a uterine artery embolism, or the microsphere comprises a chemotherapeutic agent or is mixed with a chemotherapeutic agent and the method induces a transarterial chemoembolism (TACE).

In yet another aspect, the present disclosure provides a method of treating a disease or disorder in a subject in need thereof, comprising administering to the subject a plurality of the microspheres described above. In one embodiment, the disease or disorder is selected from tendinopathy, osteoarthritis, frozen shoulder, tennis elbow (lateral epicondylitis), golfer's elbow (medial epicondylitis), pitcher's elbow (flexor tendinitis), Achilles tendinopathy, plantar fasciitis, symptomatic accessory navicular bone, hamstring tendinopathy, jumper's knee (patellar tendonitis), runner's knee (patellofemoral pain syndrome (PFPS)), pes anserine bursitis (knee pain), posterior tibial muscle tendinopathy, wrist (TFCC-Triangular FibroCartilage Complex) tendinopathy, trigger finger (stenosing flexor tenosynovitis), and haemarthrosis.

In yet another aspect, the present disclosure provides a method of rapidly degrading a microsphere in a subject, comprising administering to the subject a bail out solution, wherein a plurality of microspheres described above was previously administered to the subject and the bail out solution comprises an enzyme capable of degrading the microspheres. In one embodiment, the enzyme is complementary to the biomaterial used to form the plurality of microspheres. In one embodiment, the microsphere comprises alginate particles encapsulating alginate lyase and the enzyme is alginate lyase, the microsphere comprises pectin particles encapsulating pectinase and the enzyme is pectinase, the microsphere comprises hyaluronic acid particles encapsulating hyaluronidase and the enzyme is hyaluronidase, the microsphere comprises gelatin particles encapsulating a matrix metalloproteinase or protease and the enzyme is a matrix metalloproteinase or protease, the microsphere comprises albumin particles encapsulating peptidase and the enzyme is peptidase, the microsphere comprises collagen particles encapsulating protease and the enzyme is protease, the microsphere comprises fibrinogen particles encapsulating plasmin and the enzyme is plasmin, the microsphere comprises silk fibrin particles encapsulating protease and the enzyme is protease, the microsphere comprises starch particles encapsulating amylase and the enzyme is amylase, the microsphere comprises chitosan particles encapsulating chitosanase or lysozyme and the enzyme is chitosanase or lysozyme, the microsphere comprises agar/agarose particles encapsulating agarase and the enzyme is agarase, the microsphere comprises carrageenan particles encapsulating carrageenase and the enzyme is carrageenase, the microsphere comprises pullulan particles encapsulating pullulanase and the enzyme is pullulanase, the microsphere comprises dextran particles encapsulating dextranase and the enzyme is dextranase, the microsphere comprises b-glycan particles encapsulating b-glycanase and the enzyme is b-glycanase, the microsphere comprises cellulose particles encapsulating cellulase and the enzyme is cellulase, or the microsphere comprises lignin particles encapsulating ligninase and the enzyme is ligninase. In one embodiment, the bail out solution further comprises a divalent metal chelator.

In yet another aspect, the present disclosure provides a method of rapidly degrading a divalent metal ion crosslinked microsphere in a subject, comprising administering to the subject a bail out solution, wherein a plurality of divalent metal ion crosslinked microspheres described above was previously administered to the subject and the bail out solution comprises an anion, a phosphate buffer, or a combination thereof. In one embodiment, at least one of (i)-(iii) applies: (i) the anion comprises citrate; (ii) the phosphate buffer comprises phosphate buffered saline; and (iii) the microsphere comprises alginate particles encapsulating alginate lyase, pectin particles encapsulating pectinase, or carrageenan particles encapsulating carrageenase. In one embodiment, the microsphere comprises alginate particles encapsulating alginate lyase, pectin particles encapsulating pectinase, or carrageenan particles encapsulating carrageenase; and wherein the anion comprises citrate; or the microsphere comprises alginate particles encapsulating alginate lyase, pectin particles encapsulating pectinase, or carrageenan particles encapsulating carrageenase; and wherein the phosphate buffer comprises phosphate buffered saline.

In yet another aspect, the present disclosure provides a kit comprising: (i) a plurality of microspheres described above; and an enzyme capable of rapidly degrading the microspheres when dissolved to form a solution; or (ii) a plurality of divalent metal ion crosslinked microspheres described above; and an inorganic salt capable of rapidly degrading the microspheres when dissolved to form a solution. In one embodiment, the enzyme of (i) is complementary to the biomaterial used to form the plurality of microspheres. In one embodiment, for the plurality of microspheres of (i): the microspheres comprise alginate particles encapsulating alginate lyase and the enzyme is alginate lyase, the microspheres comprise pectin particles encapsulating pectinase and the enzyme is pectinase, the microspheres comprise hyaluronic acid particles encapsulating hyaluronidase and the enzyme is hyaluronidase, the microspheres comprise gelatin particles encapsulating a matrix metalloproteinase or protease and the enzyme is a matrix metalloproteinase or protease, the microspheres comprise albumin particles encapsulating peptidase and the enzyme is peptidase, the microspheres comprise collagen particles encapsulating protease and the enzyme is protease, the microspheres comprise fibrinogen particles encapsulating plasmin and the enzyme is plasmin, the microspheres comprise silk fibrin particles encapsulating protease and the enzyme is protease, the microspheres comprise starch particles encapsulating amylase and the enzyme is amylase, the microspheres comprise chitosan particles encapsulating chitosanase or lysozyme and the enzyme is chitosanase or lysozyme, the microspheres comprise agar/agarose particles encapsulating agarase and the enzyme is agarase, the microspheres comprise carrageenan particles encapsulating carrageenase and the enzyme is carrageenase, the microspheres comprise pullulan particles encapsulating pullulanase and the enzyme is pullulanase, the microsphere comprises dextran particles encapsulating dextranase and the enzyme is dextranase, the microspheres comprise b-glycan particles encapsulating b-glycanase and the enzyme is b-glycanase, the microspheres comprise cellulose particles encapsulating cellulase and the enzyme is cellulase, or the microspheres comprise lignin particles encapsulating ligninase and the enzyme is ligninase. In one embodiment, for the plurality of microspheres of (ii): the microspheres comprise alginate particles encapsulating alginate lyase, pectin particles encapsulating pectinase, or carrageenan particles encapsulating carrageenase. In one embodiment, the inorganic salt of (ii) releases citrate or phosphate when dissolved to form a solution.

### BRIEF DESCRIPTION OF THE SEVERAL VIEWS OF THE DRAWINGS

The foregoing summary, as well as the following detailed description of embodiments of the compositions and fluid delivery devices, will be better understood when read in conjunction with the appended drawings of exemplary embodiments. It should be understood, however, that embodiments of the present disclosure are not limited to the precise arrangements and instrumentalities shown.

FIG. 1 illustrates a general procedure for the preparation of a dehydrated and sterile composition of biologically derived microspheres of the disclosure comprising a complementary enzyme.

FIG. 2A illustrates the preparation and tailoring of properties of exemplary microspheres of the present disclosure. Alginate and alginate lyase dissolved in aqueous media. Microspheres prepared by conventional methods to gel alginate+lyase droplets by cationic crosslinking.

FIG. 2B illustrates the preparation and tailoring of properties of exemplary microspheres of the present disclosure. Microspheres are lyophilized with optional lyoprotectant to remove water and “freeze” enzyme activity, preventing premature degradation during storage. Microspheres are sterilized in this form.

FIG. 2C illustrates the preparation and tailoring of properties of exemplary microspheres of the present disclosure. Degradation properties may be controlled by varying lyase and alginate parameters and preparation conditions to produce particles of varied degradation rates from days to months depending upon indication to be treated.

FIG. 3A illustrates exemplary post-particle preparation processes and methods of use. Lyophilized alginate particles are prepared to remove water and freeze enzyme activity. Cryoprotectant protects enzyme and microsphere structure to allow shape recovery upon hydration.

FIG. 3B illustrates exemplary post-particle preparation processes and methods of use. Particles are reconstituted in aqueous media at point of use, hydrating the particle and enabling catalytic activity of the lyase.

FIG. 3C illustrates exemplary post-particle preparation processes and methods of use. Particles are prepared in an appropriate suspension for intra-arterial delivery for the designated embolization procedure (e.g., uterine fibroid embolization). When in the body, the enzyme activity is enhanced and the alginate chains are cleaved, releasing the cations, polymer chain fragments and lyase into the body where they may be resorbed or excreted.

FIG. 4A illustrates enzyme concentration dependent alginate particle degradation. A line graph of the degradation of alginate particles over time with varying enzyme concentrations is provided.

FIG. 4B illustrates enzyme concentration dependent alginate particle degradation. Images of the particles after the degradation period, and samples having varying concentrations of enzyme.

FIG. 5 illustrates enzyme concentration dependent degradation of Ca²⁺-crosslinked alginate microspheres prepared from alginate (Viscosity 144 cps@ 1% w/v of alginate, 25° C.). Alginate lyase precursor solution containing 0.25 U/ml, 0.5 U/ml and 1 U/ml of alginate lyase and 1.5% w/v of alginate. The control microspheres do not contain enzyme. Scale bar=5 mm.

FIG. 6 illustrates pH dependent regulation of enzyme conformation/activity.

FIG. 7 illustrates preparation of Ca²⁺-crosslinked alginate microspheres loaded with 5 U of alginate lyase enzyme using alginate lyase-alginate precursor solution pre-treated with (a) 0.1 M acetate buffer, pH 4.0 and (b) 0.01 M phosphate buffer, pH 6.5.

FIG. 8 illustrates microscopic images of degraded Ca²⁺-crosslinked alginate microspheres prepared from alginate lyase (AL)-alginate (Alg) precursor solution containing 5 U of AL enzyme pre-treated with (a and c) 0.1 M acetate buffer, pH 4.0 and (b and d) 0.01 M phosphate buffer, pH 6.5, in phosphate buffer at 0 and 72 hours respectively. (e) Absorbance spectra of the degraded products of Alginate-AL microspheres corresponding to (c) and (d) samples.

FIG. 9 illustrates absorbance spectra of degraded products obtained from alginate-alginate lyase (AL, 5 U) precursor solution in acetate buffer (A.B), pH 4 (Alginate-AL A.B), alginate-alginate lyase (AL) precursor solution in 0.01 M phosphate buffer (P.B), pH 6.5 (Alginate-AL P.B) and alginate lyase pre-incubated in A.B for 15 mins and mixed with alginate dissolved in 0.01M P.B ((Alginate (P.B)-AL (A.B)) for 30 mins at 1-4° C. and 37° C. respectively.

FIG. 10A illustrates ex vivo degradation studies of alginate lyase loaded calcium ion-complexed alginate particles in a liver model at 0 hours.

FIG. 10B illustrates ex vivo degradation studies of alginate lyase loaded calcium ion-complexed alginate particles in a liver model at 24 hours.

FIG. 10C illustrates ex vivo degradation studies of alginate lyase loaded calcium ion-complexed alginate particles in a liver model at 48 hours.

FIG. 11 illustrates Ca²⁺-crosslinked alginate microspheres loaded with 1 U alginate lyase enzyme and 0.5% w/v-PVP 40 KDa+0.5% w/v trehalose (A and A′) and 0.5% w/v hydroxypropyl-β cyclodextrin (B and B′), before and after lyophilization respectively.

FIG. 12 illustrates microscopic images of lyophilized Ca²⁺-crosslinked alginate microspheres loaded with 5 U alginate lyase enzyme and (a) 0.5% w/v-PVP 40 kDa+0.5% w/v trehalose and (b) 0.5% w/v hydroxypropyl-β cyclodextrin (before degradation); (c) and (d) samples are corresponding to (a) and (b) respectively, degraded in 0.01 M phosphate buffer (pH 6.5) after 72 hours of incubation at 37° C. Absorbance spectra of the degraded product of the samples (c) and (d) in 0.01 M phosphate buffer (pH 6.5) after 72 hours of incubation at 37° C. (e).

FIG. 13 illustrates in vitro biocompatibility of alginate lyase loaded-calcium ion alginate particles.

FIG. 14 illustrates post lyophilized resorbable beads rehydrated in saline at physiological conditions observed at 4 time points.

FIG. 15 is a chart showing re-establishment of flow following embolization and subsequent degradation of rehydrated resorbable alginate beads containing 0.05 U of alginate lyase in an Elastrat Liver Model.

FIG. 16 is a chart showing re-establishment of flow following embolization and subsequent degradation of rehydrated resorbable alginate beads containing 0.01 U (without (w/o) addition crosslinking in CaCl₂)), with additional crosslinking in CaCl₂) solution (0.01 U+CaCl₂), and permanent beads in the Elastrat Liver Model.

FIG. 17 is a chart showing alkaline pH dependent reversible alginate lyase activity.

FIG. 18 is a graph showing the effect of e-beam sterilization on the activity of alginate lyase enzyme (0.05 U) encapsulated into Ca²⁺-crosslinked alginate beads (beads without enzyme shown as a control).

FIG. 19 illustrates an exemplary syringe showing the compartments for the suspension medium and dried alginate microspheres. The separating membrane may be torn inside the syringe by applying pressure on the plunger, thereby reconstituting the dried alginate microsphere in the suspension medium containing calcium chloride solution.

FIGS. 20A-20C depict the degradation of pectin lyase encapsulated calcium-crosslinked pectin beads after 24 hours. FIG. 20A is an image of degraded pectin lyase-pectin beads. FIG. 20B is an image of control calcium-crosslinked pectin beads (no lyase). FIG. 20C is a bar graph of the absorbance of calcium crosslinked pectin lyase-pectin beads and the pectin-no lyase pectin beads.

FIGS. 21A-21B illustrate the enzyme concentration dependent degradation of Ca²⁺-crosslinked alginate microspheres prepared from alginate (Viscosity 160 cps @ 2% w/v of alginate, 25° C.). The alginate lyase precursor solution contained 0.15 U/mL, 0.5 U/mL, and 1 U/mL of alginate lyase. The control (permanent) microspheres do not contain enzyme.

FIGS. 22A-22D provide images of a porcine kidney that has been injected with a reversible embolic liquid. FIG. 22A is an image of a kidney segment immediately after injection of 1.8 mL of alginate microspheres that do not encapsulate alginate lyase (the embolic liquid). FIG. 22B shows partial revascularization of flow within 10 minutes after the injection of an alginate lyase bail out solution. FIG. 22C shows an image where, after 2 hrs of occlusion, the alginate lyase bail out solution was injected to reverse the occlusion and restore original flow. FIG. 22D shows complete revascularization after 24 hrs in the porcine kidney model.

FIGS. 23A-23E show the reperfusion of a porcine kidney embolized with 0.15 U and 0.0875 U alginate lyase loaded resorbable alginate microspheres. FIGS. 23A-23D show the opening of blood vessels over 20 hours for the 0.15 U and 0.0875 U alginate lyase loaded alginate microspheres. FIG. 23E shows the parenchyma of embolized sites in the kidney.

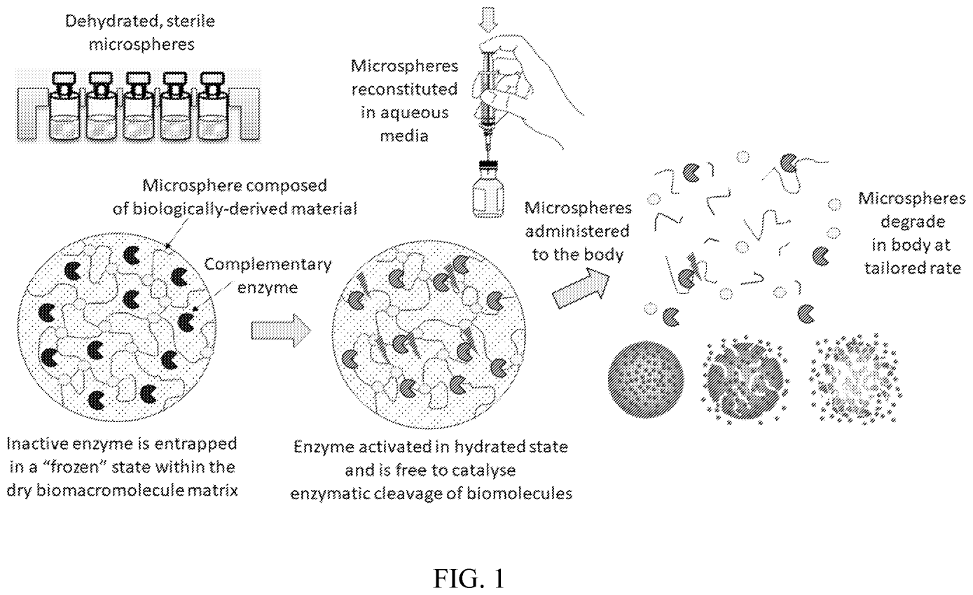

### DETAILED DESCRIPTION

### Overview

Generally, the present disclosure provides dehydrated and/or sterilized compositions comprising a biologically derived particle containing an enzyme that can act on the biologically derived particle. In one embodiment, the enzyme that can act on the biologically derived particle is its complementary enzyme. In one embodiment, the enzyme acts to control the degradation of the biologically derived particle. In one embodiment, the biologically derived particle is a microparticle.

In one embodiment, the biologically derived particle comprises a protein or glycoprotein biomaterial. Exemplary biologically derived particles comprising proteins or glycoproteins include, but are not limited to, an albumin particle, a collagen particle, a gelatin particle, a fibrinogen particle, or a silk fibrin particle, each particle containing an enzyme that can act on the biologically derived particle. In one embodiment, the biologically derived particle enzyme combination is albumin containing peptidase, collagen or gelatin containing protease, gelatin containing a matrix metalloproteinase, fibrinogen containing plasmin, or silk fibrin containing protease. In another embodiment, the biologically derived particle comprises a polysaccharide. Exemplary biologically derived particles comprising polysaccharides include, but are not limited to, an alginate particle, a hyaluronic acid particle, a pectin particle, a starch particle, a chitosan particle, an agar/agarose particle, a carrageenan particle, a pullulan particle, a dextran particle, a b-glycan particle, a cellulose particle, or a lignin particle, each particle containing an enzyme that can act on the biologically derived particle. In one embodiment, the biologically derived particle enzyme combination is alginate containing alginase (alginate lyase enzyme), hyaluronic acid containing hyaluronidase, pectin containing pectinase, starch containing amylase, chitosan containing chitosanase or lysozyme, agar/agarose containing agarases, carrageenan containing carrageenase, pullulan containing pullulanase, dextran containing dextranase, b-glycans containing b-glycanses, cellulose containing cellulase, or lignin containing ligninase.

The enzyme containing biologically derived particles of the present disclosure can be administered to a subject to treat a disease or disorder in the subject. Exemplary diseases or disorders include, but are not limited to, osteoarthritis (e.g., knee osteoarthritis, finger osteoarthritis); sports medicine diseases/disorders (e.g., frozen shoulder, tennis elbow (lateral epicondylitis), golfer's elbow (medial epicondylitis), pitcher's elbow (flexor tendinitis), Achilles tendinopathy, plantar fasciitis, symptomatic accessory navicular bone, hamstring tendinopathy, jumper's knee (patellar tendonitis), runner's knee (patellofemoral pain syndrome (PFPS)), pes anserine bursitis (knee pain), posterior tibial muscle tendinopathy, wrist (TFCC-Triangular FibroCartilage Complex) tendinopathy, trigger finger (stenosing flexor tenosynovitis)); and orthopedic diseases/disorders (e.g., haemarthrosis). In another embodiment, the enzyme containing biologically derived particles of the present disclosure are used in an embolization procedure in a subject in need thereof. Exemplary embolization procedures include, but are not limited to, those performed in combination with TACE (wherein the particles of the disclosure maximize therapeutic effect by ensuring the chemo-agent stays in situ for period of absorption), prostate arterial embolization, and uterine artery embolization.

In one embodiment, the enzyme containing biologically derived particles of the present disclosure are administered to the subject to treat tendinopathy in the subject. In another embodiment, the enzyme containing biologically derived particles of the present disclosure are administered to a subject suffering from a disease or disorder that would benefit from treatment with a temporary embolization agent.

Alginate based liquid embolic agents have been considered as a promising alternative to conventional embolic agents. Pure forms of alginate are highly biocompatible, and their gelling properties can be controlled. They are naturally-occurring polysaccharide copolymers composed of randomly 1-4 linked β-D-mannuronic acid (M-block)-α-L-guluronic (G block) of various M:G ratios that are commonly found in various seaweeds. In the prior-art disclosure, alginate is dissolved in the contrast agent iohexol (to impart radiopacity) and is gelled into hydrocoil form which solidifies in the presence of calcium chloride solution due to ionic crosslinking of the carboxylate groups of the polysaccharide residues with Ca²⁺. All of these components were mixed simultaneously at the treatment site to create an in situ mass of gel. This gel may be subsequently dissolved using a mixture termed EmboClear by the inventors, which is a mixture of alginate lyase enzyme and EDTA (Ethylenediaminetetraacetic acid). The enzyme cleaves the polysaccharide chains at the glycosidic bond via a β-elimination mechanism and the EDTA de-complexes the ionic crosslinks by scavenging the Ca²⁺ by chelation. This dissolution agent was administered at the site of the embolus and it completely cleared the occluded vessel within a few minutes. This invention addresses some aspects of the selective degradation of the embolic agent but poses a few complications.

Firstly, the procedure to degrade the EmboGel using Emboclear solution introduces additional risk to the patient, as they must undertake additional post embolization procedures. Moreover, depending upon the time interval desired between formation of the embolus and its dissolution, this could involve rescheduling the patient for a second visit and all the associated costs for a re-catheterisation procedure. Secondly, in some cases such as aneurysm therapy, the alginate gel could migrate to the parent artery during injection or after the post-embolization procedure which may cause non-specific vessel occlusion (see, e.g., Barnett, et al., “A selectively dissolvable radiopaque hydrogel for embolic applications”; and U.S. Pat. No. 9,220,761). Similar complications would be expected with other biologically derived particles containing an enzyme that can act on the biologically derived particle.

Barnett et al. demonstrate that an alginate-based embolic material can be degraded within the body by application of an alginate lyase based composition. Purified alginate is dissolved in the contrast agent iohexol (to impart radiopacity) and is gelled into hydrocoil form which solidifies in the presence of calcium chloride solution due to ionic crosslinking of the carboxylate groups of the polysaccharide residues with Ca²⁺. All of these components were mixed simultaneously at the treatment site to create an in situ mass of gel. This gel may be subsequently dissolved using EmboClear.

In U.S. Pat. No. 9,220,761, non-specific migration of degraded/disintegrated alginate gels to other parts of the body predominantly occurs due to instant/uncontrolled degradation/disintegration of EmboGel by the EmboClear, causing generation of particulates of various size and that are unable to be reabsorbed before they are distributed to off-target distal locations at which the Emboclear is ineffective due to dilution. If EmboGel is loaded with a bioactive agent/drug, it requires the separate administration of EmboClear dissolution agent in order to afford degradation-controlled release kinetics.

Boyan et al. report a method and composition of alginate particles consisting of alginate lyase and stem cells (see, i.e., PCT Publication No. WO 2012/071527 A2). Depending on the concentration of enzyme incorporated, proteins secreted by stem cells or stem cells can be delivered into the body. In contrast with various embodiments of the present disclosure, the compositions of Boyan are incapable of being lyophilized and sterilized without killing the stem cells therein. In Boyan, Ca²⁺ crosslinked alginate particles are used to encapsulate alginate lyase and stem cells for the sustained release of proteins and stem cells. In this method, the varying amount of alginate lyase along with stem cells are mixed with alginate of different molecular weight for 1 minute at 1-4° C. and gelled in a calcium chloride bath to obtain self-degradable stem cell-encapsulated calcium crosslinked alginate microspheres. In order to release the stem cells and their secreted proteins, these particles were suspended in saline at 37° C. to activate the alginate matrix-degrading catalytic activity of the alginate lyase enzyme. Furthermore, the alginate particles loaded with cells were processed with DMSO for cryopreservation in liquid nitrogen. This report provided insight into controlled degradation of alginate particles, but there are many drawbacks for using this method to produce scalable self-degradable alginate particles for embolic applications. It was observed that the reduction of temperature to 1-4° C. does not completely cease the degrading activity of the enzyme. Furthermore, the incubation period of the enzyme with alginate will be higher, if the production of these particles needs to scale up. This reduces the viscosity of the alginate, thereby reducing the encapsulation of the alginate lyase enzyme and also poses a challenge in obtaining uniformly shaped particles. Likewise, any proposed encapsulation of bioactive agents (such as anti-inflammatory and anticancer) would also be reduced. Furthermore, the method to encapsulate bioactive agents and post-particle preparation processes such as lyophilization and sterilization of degradable alginate particles were not considered. For developing self-degrading particles, it is important to enable storage for a longer time period in dried and sterile form, that can be reconstituted at the point of use, and become activated upon introduction into the body. Therefore, there is a need for self-degrading particles that can degrade or exhibit predictable resorption rates without creating any non-specific occlusion in vivo, act as a vehicle for releasing a bioactive agent, and remain stable for a long time period under desired storage conditions.

Furthermore, Kunjukunju, et al. reported alginate lyase aggregates of various size (10-300 μm) and shape using ammonium sulfate (see, e.g., Kunjukunju, et al., “Cross-linked enzyme aggregates of alginate lyase: A systematic engineered approach to controlled degradation of alginate hydrogel.” International Journal of Biological Macromolecules 115 (2018): 176-184). These aggregates were crosslinked using glutaraldehyde to produce insoluble catalytically active alginate lyase aggregates. The resultant crosslinked aggregate was encapsulated in an alginate hydrogel to achieve its controlled degradation. However, the method described in this report may not be suitable to enable the preparation of a self-degrading particle of the present disclosure per se.

Firstly, it would not be possible to produce self-degrading particles of the desired size, as the size and polydispersity of the described aggregates of the enzyme could not be encapsulated. Secondly, the process described crosslinking the enzyme aggregates with glutaraldehyde which is a toxic agent that should be avoided in the preparation of compositions intended for use in the human body. Thirdly, the authors did not report any other methods to control the degradation of the biologically derived particle, such as molecular weight or viscosity of biomaterial used to make the particle (e.g., sodium alginate for an alginate biologically derived particle), pre-treatment of the enzyme using modifiers (metal ions) or other physiochemical parameters such as pH and temperature or to improve the encapsulation efficiency of the enzyme. Lastly, no work has been performed to achieve the storage and shelf life of the resulting aggregates.

The present disclosure provides compositions and methods for making self-degradable crosslinked microspheres, wherein the microspheres are biologically derived and loaded with an enzyme that acts on the microsphere. In one embodiment, the microspheres are further loaded with bioactive agents and cryoprotectants. This allows the loading or encapsulation of the desired concentration of enzyme and bioactive agents to obtain the tailored degradation of microspheres under physiological conditions. There are many advantages of the present disclosure over the existing self-degrading particles and prior-art alginate-based systems:
1. Pre-treating the enzyme under a combination of different conditions (pH, temperature, and metal ion inhibitor) that may reversibly inhibit the catalytic degrading activity allows the controlled loading of the enzyme into biologically derived particles. This strategy provides the predictable and desired degradation rate of biologically derived particles which are of prime importance for applications which require the temporary use of self-degrading particles, such as embolic applications. Using a combination of different conditions to regulate the loading of the enzyme into the biologically derived particles has not been described for existing self-degrading particles and prior-alginate-based systems.
2. The pre-treatment of the enzyme reversibly inhibits the enzyme activity, thereby ceasing the exposure of the biologically derived particle to the active form of the enzyme for the desired length of time. This strategy may allow the scale-up production of these microspheres without prematurely degrading the biologically derived particle which encapsulates the enzyme.
3. The self-degrading nature of the enzyme containing biologically derived microspheres ensures any by-products or particulates can be reabsorbed and ultimately excreted through the kidneys. Therefore, the risk of non-specific occlusion of blood vessels is minimized.
4. The self-degradable biologically derived microspheres can also be loaded with anti-inflammatory agents, for example hyaluronic acid. The sustained release of the anti-inflammatory agent in the subject may alleviate pain in the subject. For example, sustained release of an anti-inflammatory agent at the site of embolization may alleviate the neuropathic pain that could arise from chronic inflammation.
5. The composition of self-degradable biologically derived microspheres also consists of cryoprotectants. The inclusion of cryoprotectant allows the lyophilization and subsequent sterilization without affecting the enzyme activity. These post-preparation processing steps of microspheres result in a sterile composition that can be stored for a length of time and reconstituted at the point of use to re-activate the enzyme before administration into the body.

In addition to divalent-metal ion crosslinking, photopolymerization methods may be used to prepare self-degradable biologically derived particle compositions with the same properties as discussed above. This method may further improve the calibrated degradation of the biologically derived microspheres.

### Definitions

As used herein, the term “a,” “an,” or “the” generally is construed to cover both the singular and the plural forms.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” may refer to a range of +20%, +10%, or +5% of a given numeric value.

The term “substantially” as used herein may refer to a majority of, or mostly, as in at least about 50%, 60%, 70%, 80%, 90%, 95%, 96%, 97%, 98%, 99%, 99.5%, 99.9%, 99.99%, or at least about 99.999% or more.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The term “therapeutically effective amount” may generally refer to the amount (or dose) of a compound or other therapy that is minimally sufficient to prevent, reduce, treat or eliminate a condition, or risk thereof, when administered to a subject in need of such compound or other therapy. In some instances, the term “therapeutically effective amount” may refer to that amount of compound or other therapy that is sufficient to have a prophylactic effect when administered to a subject. The therapeutically effective amount may vary; for example, it may vary depending upon the subject's condition, the weight and age of the subject, the severity of the disease condition, the manner of administration (e.g., subcutaneous delivery) and the like, all of which may be determined by one of ordinary skill in the art.

As used herein, “treating” or “treat” includes: (i) preventing a pathologic condition from occurring (e.g., prophylaxis); (ii) inhibiting the pathologic condition or arresting its development; (iii) relieving the pathologic condition; and/or (iv) diminishing symptoms associated with the pathologic condition.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the disclosure is contemplated. Supplementary active ingredients may also be incorporated into the compositions.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

### Compositions & Methods

### Crosslinking Biologically Derived Microspheres

The present disclosure relates to the loading of an enzyme into a biomaterial that can be gelled or crosslinked using any of the conventional methods used in the art to induce covalent, physical, ionic crosslinks, or a combination thereof, depending upon the biomaterial being used, to form an enzyme-loaded biologically derived microsphere. In one embodiment, a metal ion crosslinking agent is used (e.g., a divalent metal ion). In another embodiment, a homo-bifunctional crosslinking agent is used (e.g., disuccinimidyl suberate, succinimidyl tartrate, dithiobis succinimidyl propionate, bismaleimidoethane, dithiobismaleimidoethane, etc). In yet another embodiment, a heterobifunctional crosslinking agent is used (e.g., MDS (m-Maleimidobenzoyl-N-hydroxysuccinimide ester), GMBS (N-γ-Maleimidobutyryloxysuccinimide ester), EMCS (N-(ε-Maleimidocaproyloxy) succinimide ester), sulfo-EMCS (N-(ε-Maleimidocaproyloxy) sulfo succinimide ester, and their derivatives). In yet another embodiment, crosslinks are formed by thermogelation (e.g., via gelation of gelatin, agar, pectin, and other such biopolymers). In yet another embodiment, crosslinks are formed by light activated photo-polymerization or photo-crosslinking (e.g., using aryl-azides, diazirines, and their derivatives). The crosslinked microspheres of the present disclosure can then be rapidly dried and sterilized.

In one embodiment, the biologically derived microsphere containing an enzyme is crosslinked using a heterobifunctional agent. In this embodiment, a biomaterial comprising an amine or carboxyl functional group can be crosslinked using EDC-NHS click chemistry or any of the heterobifunctional agents as mentioned above to form amide or other covalently crosslinked microspheres.

In another embodiment, the biologically derived microsphere containing an enzyme is crosslinked using thermogelation. In this embodiment, the biomaterial is warmed to melt it, the enzyme is added, the resulting mixture is emulsified in oil to form droplets, and the droplets are cooled to form microspheres. In one embodiment, an aqueous solution of agarose is used as the biomaterial, wherein the solution is warmed to 40° C., melting the agarose, an enzyme is added, the resulting mixture is emulsified in oil to form droplets, and the droplets are cooled to form microspheres.

In order to make divalent metal ion crosslinked biologically derived microspheres loaded with an enzyme that acts on the microsphere, the enzyme is mixed with a biomaterial capable of being crosslinked by metal ions (precursor solution) and dripped into a divalent metal ion gelling bath.

In the case of photo-crosslinked biologically derived microspheres loaded with enzyme, a biomaterial comprising a photo-crosslinkable moiety is mixed with the enzyme and photoinitiators (precursor solution), which can be either drop casted or injected into different liquid containing surfactant or oils (mono- or double emulsion) using a microfluidics platform for instance, to form droplets. These droplets are irradiated with near UV wavelength (200 nm-400 nm) light for various times. Upon irradiation, the crosslinking of the biomaterial comprising a photo-crosslinkable moiety occurs to form the enzyme loaded biologically derived microspheres. In one embodiment, the photo-crosslinkable moiety is an acrylate or methacrylate moiety.

In one embodiment, the microspheres of the present disclosure can also encapsulate anti-inflammatory agents. In one embodiment, the anti-inflammatory agents are during the preparation of the containing biologically derived microspheres and then crosslinking the microspheres with the above-mentioned crosslinking procedures. The degradation of these crosslinked enzyme containing biologically derived particles can be controlled by the composition of precursor solution, the composition of the gelling bath, and the method of preparing these particles.

In one embodiment, the biologically derived particle comprises a protein or glycoprotein, for example, an albumin particle, a collagen particle, a gelatin particle, a fibrinogen particle, or a silk fibrin particle. In one embodiment, the biologically derived particle enzyme combination is albumin containing peptidase, collagen or gelatin containing protease, gelatin containing a matrix metalloproteinase, fibrinogen containing plasmin, or silk fibrin containing protease. In another embodiment, the biologically derived particle comprises a polysaccharide, for example, an alginate particle, a hyaluronic acid particle, a pectin particle, a starch particle, a chitosan particle, an agar/agarose particle, a carrageenan particle, a pullulan particle, a dextran particle, a b-glycan particle, a cellulose particle, or a lignin particle. In one embodiment, the biologically derived particle enzyme combination is alginate containing alginase (alginate lyase enzyme), hyaluronic acid containing hyaluronidase, pectin containing pectinase, starch containing amylase, chitosan containing chitosanase or lysozyme, agar/agarose containing agarases, carrageenan containing carrageenase, pullulan containing pullulanase, dextran containing dextranase, b-glycans containing b-glycanses, cellulose containing cellulase, or lignin containing ligninase.

In one embodiment, the biologically derived particle is an alginate microsphere loaded with alginate lyase. Therefore, in one embodiment, the biomaterial is sodium alginate or methacrylate-alginate loaded alginate lyase enzyme. In one embodiment, sodium alginate is used as the biomaterial when the crosslinking agent is a divalent metal ion. In another embodiment, methacrylate-alginate is used as the biomaterial for crosslinking by light activated photopolymerization.

Compositions and Methods of Preparing Divalent Metal Ion Crosslinked Self-Degrading Biologically Derived Microspheres Loaded with an Enzyme

### Composition of Precursor Solution

The degradation of enzyme loaded crosslinked biologically derived particles can be controlled by the composition of precursor solution. In one embodiment, the concentration of the enzyme loaded into biologically derived microspheres can control the degradation of the crosslinked biologically derived particles. In one embodiment, the enzyme may be pre-treated with pH, temperature, metal ion inhibitors, organic and inorganic competitive inhibitors, non-inhibitors, end-product inhibitors, or a combination thereof. In one embodiment, pre-treatment of the enzyme can control the degradation of the enzyme loaded crosslinked biologically derived particles.

In the precursor solution, the enzyme causes breakdown of the biomaterial which can impact the encapsulation of the enzyme in the biologically derived particles. This also reduces the initial viscosity of enzyme solution, which is important for maintaining both encapsulation efficiency within the biologically derived particles and also for obtaining particles of desired size and shape. The enzyme may therefore be pre-treated at different pH, low temperature and/or exposure to metal ion inhibitors, before adding the precursor mix to the divalent metal ion gelling bath for crosslinking.

The optimum catalytic activity of alginate lyase or alginase used in the present disclosure is observed at a pH ranging from 6.8 to 7.5. To prevent the initial degradation of the biomaterial during the preparation of the enzyme loaded biologically derived particles, the pH of the enzyme-biomaterial solution may be reduced to 3.0. To carry out this process, sodium acetate-acetic acid buffer, of ionic strength <1 M, preferably <0.1 M and most preferably <0.01 M with a pH range 3.7-5.6 is used. In addition, the desired pH (pH 6.5 to 3.0) of the solution may also be achieved using sodium hydroxide (>1 M to <0.01 M) or hydrochloric acid (>1 M to <0.01 M). This results in the reduction or ceasing of the enzyme catalytic activity. This regulation of the catalytic activity may be attributed to the unfolding of 3D conformation of enzyme. The ceased catalytic activity of the enzyme may be reversed/activated by exposing the enzyme loaded biologically derived particles to an aqueous environment having a pH of 6.5 to 7.5. The preferred buffers for reversing the activity of the enzyme are phosphate buffers. The preferred ionic strength of the phosphate buffer is 0.01 M with a pH range of 6.5 to 7.5 at 20° C. The desired pH (pH 6.5 to 7.5) of the solution may also be achieved using sodium hydroxide (>1 M to <0.01 M) or hydrochloric acid (>1 M to <0.01 M). Additionally, saline or deionized water, or an aqueous solution having a pH of between 6.5 to 7.5 may also be used.

In combination with changing the pH of the solution, the temperature of the individual components of the precursor solution before mixing may be maintained at 1-4° C. to inhibit the degradation of biomaterial. The temperature of precursor solution after mixing the individual components may be maintained at 1-4° C. to inhibit the degradation of biomaterial. Note that the temperature will also influence the viscosity of the solution.

In addition to changing the pH and temperature of the precursor solution, the enzyme may be pre-treated with the metal ion inhibitors such as Cu²⁺, Zn²⁺, and Fe³⁺. These metal ions can inhibit the activity of the enzyme (Inoue, et al., “Functional identification of alginate lyase from the brown alga Saccharina japonica,” Sci. Rep. 2019; 9:1-11).

Therefore, a combination of the abovementioned approaches may be used to inhibit (reversibly or partially) the enzyme in the precursor solution. This may enhance the loading of enzyme into the biologically derived particles without degrading the particle matrix. The combination of these approaches has not been adopted in the cited reports.

Other factors which improve the robustness of the biomaterial are choice of divalent ions and molecular weight/viscosity of biomaterial. The molecular weight or viscosity of the biomaterial also affect the mechanical properties of the biologically derived particle (Farrés, et al., “Formation kinetics and rheology of alginate fluid gels produced by in-situ calcium release,” Food Hydrocolloids 40 (2014): 76-84).

To achieve the rapid degradation of enzyme loaded divalent metal ion complexed biologically derived particles, a biomaterial having low molecular weight/low viscosity may be used in the precursor solution. In one embodiment, use of a biomaterial having low molecular weight/low viscosity results in a biologically derived particle which degrades in >20 minutes to <=4 hours. In one embodiment, use of a biomaterial having low molecular weight/low viscosity results in a biologically derived particle which degrades in less than about 12 hours, less than about 10 hours, less than about 8 hours, less than about 6 hours, less than about 4 hours, less than about 3 hours, less than about 2 hours, or less than about 1 hour. To achieve intermediate (5 days to 30 days) or slow (>30 days) degradation periods, a biomaterial having high molecular weight/high viscosity may be used, which can be considered to be >70 mPas.

The activity (Units, U) of treated (pH, temperature, and/or metal ion inhibitor exposed) enzyme mixed with biomaterials of different molecular weight in the precursor solution also regulates the degradation of divalent metal ion-crosslinked biologically derived microspheres. For the rapid degradation of biologically derived microspheres (>20 minutes to <=4 hours), the preferred activity of the enzyme may range from 0.0075 U/mg to 0.25 U/mg of biomaterial. To get intermediate (5 days to 30 days) or slow (>30 days) degradation periods, the preferred range of enzyme activity may be <0.005 U/mg to 0.025 U/mg of biomaterial and <0.0025 U/mg of biomaterial respectively.

In one embodiment wherein the biomaterial is alginate, the predetermined molecular weight of alginate and ratio of M (β-D-mannuronic acid) and G (α-L-guluronic acid) blocks (M/G) can control the self-degradation of the resulting alginate microspheres. Particularly, the G block has more affinity toward divalent cations as compared to the M block due to the geometry of the carboxylate residues. Alginate contains a large variation in the M and G content, and also possesses the variation in the sequence structures (G block, M block and MG block) (Ramos, et al., “Effect of alginate molecular weight and M/G ratio in beads properties foreseeing the protection of probiotics,” Food Hydrocoll. 2018; 77:8-16). In general, the alginate with a higher G content relative to M content (lower M/G ratio) when crosslinked with cations gives more mechanically robust structures/capsules with low permeability and greater resistance to enzyme degradation, when compared to the alginate with higher M/G ratio.

In one embodiment, the enzyme loaded biologically derived particle is an alginate lyase-loaded divalent metal ion complexed alginate particle. To achieve the rapid degradation (>20 minutes to <=4 hours) of these particles, lower G content alginate (e.g., higher M:G ratio) having low molecular weight/low viscosity may be used in the precursor solution. In certain embodiments, the purified alginate contains more than 50% M content (β-D-mannuronic acid). The percentage of M content in the purified alginate maybe 50% and 80%, 55%-75% and 60%-80%. To get intermediate (5 days to 30 days) or slow (>30 days) degradation periods, the higher G content alginate (e.g., the lower M:G ratio) having high molecular weight/viscosity may be used. In certain embodiments, the purified alginate contains more than 50% G content (α-L-guluronic acid). The percentage of G content in the purified alginate maybe 50% and 80%, 55%-75% and 60%-80%. The preferred M content may be in the range of about 55% to about 65% to obtain particle degradation in the short and intermediate time periods. Although not wishing to be limited by theory, it is believed that G residues bind metal ions tighter than M residues. Therefore, a higher M content results in microspheres that are likely to be less tightly crosslinked than corresponding microspheres with a higher G content and would thus degrade more quickly as it is easier to displace the crosslinking ions and open the structure.

In one embodiment wherein the biomaterial is alginate, the average molecular weight of alginate polymers may be >200 kDa, preferably >100 kDa and most preferably >30 kDa. The viscosity of 1% alginate solution at 20° C. may have a range >25 mPa-s, preferably <1000 mPa-s for the preparation of rapid and slow degrading alginate lyase loaded divalent metal ion complexed alginate particles.

In one embodiment wherein the self-degrading biologically derived particle is an alginate particle loaded with alginate lyase, the activity (Units, U) of treated (pH, temperature, and metal ion inhibitor exposed) alginate lyase enzyme mixed with alginate of different molecular weight and M/G ratio (M (β-D-mannuronic acid) and G (α-L-guluronic acid) blocks (M/G) ratio) in the precursor solution also regulates the degradation of divalent metal ion-crosslinked alginate microspheres. The activity of the alginate lyase enzyme may range from 0.025 U/mg to 1 U/mg of alginate. For the rapid degradation of alginate microspheres (>20 minutes to <=4 hours), the preferred activity of the enzyme may range from 0.0075 U/mg to 0.25 U/mg of alginate. To get intermediate (5 days to 30 days) or slow (>30 days) degradation periods, the preferred range of enzyme activity may be 0.005 U/mg to 0.0025 U/mg of alginate and <0.0025 U/mg to 0.005 U/mg of alginate respectively. Although not wishing to be limited by theory, it is believed that the higher the activity of the enzyme (there more there is encapsulated), the faster the enzyme will breakdown the microsphere.

Use of Bioactive Agents Encapsulated within the Biologically Derived Particles

In one embodiment, the self-degrading enzyme loaded biologically derived particles of the present disclosure can encapsulate a bioactive agent, such as an anti-inflammatory agent (including NSAIDs and non-NSAIDs), a chemotherapeutic agent, a corticosteroid, or an antioxidant, which functions to provide localized pain relief when administered to a subject. Exemplary NSAID anti-inflammatory agents include, but are not limited to, ibuprofen, flurbiprofen, meloxicam, nabumetone, etodolac, ketorolac, ketoprofen, diflusinal, naproxen, diclofenac, celecoxib, mefenamic acid, etoricoxib, indomethacin, and aspirin. Exemplary non-NSAID agents include, but are not limited to, arnica, curcurmin, bromelain, and acetaminophen. Exemplary corticosteroids include, but are not limited to, methylprednisolone, dexamethasone, triamcinolone, betamethasone, beclomethasone, and hydrocortisone. Exemplary antioxidants include, but are not limited to, glutathione, a-tocopherol, ergothioneine, N-acetylcysteine, ascorbic acid, vitamin A, vitamin E, allopurinol, melatonin, resveratrol, bucillamine, turmeric, lycopene, dihydrolipoic acid, cobalamins, flavonoids, quercetin, ebselen, and edaravone.

Previous reports demonstrated that high molecular weight hyaluronic acid (100-500 kDa) or breakdown products of hyaluronic acid display anti-inflammatory and immunosuppressive activity. Therefore, the use of hyaluronic acid encapsulated within the enzyme loaded biologically derived particles of the present invention could provide pain relief when administered to a subject. Similarly, other anti-inflammatory agents or antioxidants would also be expected to provide pain relief when administered to the subject.

To the precursor solution mentioned previously mentioned, bioactive agents such as high molecular weight hyaluronic acid may be added. This involves the addition of the bioactive agent at 1% wt to 20% wt of biomaterial concentration in the precursor solution. On crosslinking in the divalent metal ion-gelling bath, the bioactive agent may be encapsulated. In one embodiment, high molecular weight hyaluronic acid is encapsulated in the enzyme loaded-divalent metal ion crosslinked biologically derived microspheres described elsewhere herein.

In one embodiment, the bioactive agent provides localized pain relief in a subject suffering from tendinopathy. In another embodiment, the bioactive agent provides localized pain relief in a subject during an embolization medical intervention. The encapsulated bioactive agent may be released at the site of embolization or the site of tendinopathy or osteoarthritis due to the degradation of the enzyme loaded biologically derived microspheres. For instance, many embolization medical interventions cause neuropathic pain which may be relieved by the use of hyaluronic acid or its breakdown products, alginate and its breakdown products, NSAIDs, non-NSAIDs, corticosteroids, and antioxidants. These pharmacologically active molecules may alleviate pain arises due to tendinopathy and osteoarthritis.

In another embodiment, the self-degrading enzyme loaded biologically derived particles of the present disclosure are formed from a polysaccharide biomaterial, yielding a polysaccharide particle which encapsulates an enzyme that can act on the polysaccharide, wherein the particle does not encapsulate an anti-inflammatory agent or antioxidant. In one embodiment, the enzyme breaks down the polysaccharide particle, forming oligosaccharides which have an anti-inflammatory effect. Therefore, in some embodiments, the particles of the disclosure are able to provide an anti-inflammatory effect and/or pain relief when administered to a subject even when the particles of the disclosure do not encapsulate an anti-inflammatory agent or antioxidant.

### Composition of Gelling Bath

The precursor enzyme-biomaterial (appropriate molecular weight and optionally M/G ratio, for a sodium alginate biomaterial and alginate lyase enzyme) solution under the appropriate conditions (low temperature, pH, or metal ion) as described elsewhere herein needs gelling in a divalent metal ion bath to form microspheres of the present disclosure. In some embodiments, a bioactive agent is added to the precursor enzyme-biomaterial solution and the resulting mixture is gelled in a divalent metal ion bath to form microspheres. The particle size obtained may be >40 μm, <200 μm, but is <2000 μm. To reduce the degradation of divalent crosslinked biologically derived microspheres, the temperature and pH of the gelling bath may be maintained at 1-4° C. and 3.5 to 4.0 respectively. The pH may be maintained using the buffers with ionic strength mentioned above. The composition and condition of the gelling bath are important to make desired self-degrading biologically derived particles. The divalent metal ion component of the gelling bath composition may be selected from the group consisting Cu²⁺, Ba²⁺, Sr²⁺, Ca²⁺, Co²⁺, Ni²⁺, Mn²⁺, and Mg²⁺ (Lee, et al., “Alginate: properties and biomedical applications,” Progress in polymer science 37, no. 1 (2012): 106-126; and Brus, et al., “Structure and dynamics of alginate gels cross-linked by polyvalent ions probed via solid state NMR spectroscopy,” Biomacromolecules 18, no. 8 (2017): 2478-2488). Divalent cation choice may also influence crosslinking of the biomaterial matrix which forms the outside of the biologically derived particles. In one embodiment, the biologically derived particles comprise alginate particles loaded with alginate enzyme wherein the binding strength of divalent metal ion with alginate is given in decreasing order Cu²⁺>Ba²⁺>Sr²⁺>Ca²⁺>Co²⁺>Ni²⁺>Mn²⁺>Mg²⁺. The preferred metal cations are Ba²⁺ and Ca²⁺. These metal ions may be used at different concentrations ranging from 0.1% w/v to 10% w/v. The preferred concentration of divalent metal ion may be 0.001% w/v to 2% w/v. In another embodiment, the biologically derived particles comprise pectin loaded with an enzyme that acts on the pectin, such as pectinase, wherein the binding strength of divalent metal ion with pectin depends on the pectin source. In one embodiment, the pectin is nopal pectin with a divalent metal ion binding strength given in decreasing order of Ca²⁺>Cu²⁺>Zn²⁺>Cr³⁺>Ni²⁺>Pb²⁺>Cd²⁺. In one embodiment, the pectin is from citrus and sugar beet sources with a divalent metal ion binding strength given in decreasing order of Cu²⁺≈Pb²⁺>Zn²⁺>Cd²⁺≈Ni²⁺>Ca²⁺. These metal ions may be used at different concentrations ranging from 0.1% w/v to 10% w/v.

### Water Content and Sterilization of Biologically Derived Particles

The shelf-life of the self-degrading biologically derived microspheres loaded with an enzyme may be improved through dehydrating the microspheres such that they are substantially free of water and sterilizing the dehydrated microspheres.

The microspheres can be dehydrated using any technique known to a person of skill in the art. In one embodiment, the microspheres are lyophilized. In another embodiment, the microspheres are dehydrated or dried using super-critical CO₂. In this method, the crosslinked microspheres are treated with ethanol, acetone, or ethanol:water mixture ranging from 10:90 to 98:2 to displace and substitute water. The treated crosslinked particles are further processed by super-critical CO₂. The process involves the removal of the solvent in a stepwise manner using pressurized liquid CO₂. Here, the particles are placed in a stainless-steel pressurized container and exposed to liquid CO₂ and heat at a particular temperature (preferably about 35° C. to about 45° C.) for 3 to 4 hours, before de-pressuring the chamber to remove the solvents. A cryoprotectant (e.g., trehalose) can be employed for the preservation of enzyme only.

In one embodiment, the microspheres are dried such that the water content of each microsphere is less than about 15%, less than about 12%, less than about 10%, less than about 8%, less than about 6%, less than about 4%, or less than about 1% by mass. In one embodiment, the water content of each microsphere is between about 1% and about 10% by mass. Although not wishing to be limited by theory, it is believed if the water content of the microsphere is too high, the enzyme structure is denatured by the sterilization, while if the water content is too low, the microspheres do not hydrate properly and also the enzyme activity is also affected (a little water is needed to maintain the enzyme 3D conformation and activity).

For these post-preparation processes, cryoprotectants may be added into precursor solution (described elsewhere herein) and gelling bath. The addition of cryoprotectants is important in many ways. Firstly, it helps in maintaining the sphericity and mechanical robustness of the enzyme loaded biologically derived particles during the dehydration process. Secondly, it preserves the 3D conformation of the enzyme at extremely low temperatures and freezing cycles used during lyophilization, thereby preserving the enzyme activity. Recovery of microsphere shape post lyophilization was considered as an issue. This is not unexpected, as high-water content gels shrink during lyophilization and often do not re-establish their original shape upon rehydration. The addition of cryoprotectants such as sugars and polymers can compensate for this, to help maintain the porous structure during sublimation of the internal water within the structure. Also, it has been observed that the residual activity of the enzyme reduced significantly when the lyophilization of the enzymes was performed without the addition of the cryoprotectants/cryopreservation medium. Thus, the use of cryoprotectants also assists the rapid recovery of shape upon reconstitution in aqueous medium and enables retention of the functionality of the active ingredients including enzymes.

The microspheres can be sterilized using any technique known to a person of skill in the art. In one embodiment, the microspheres are sterilized using irradiation. In one embodiment, the irradiation is e-beam irradiation. In another embodiment, the irradiation is gamma irradiation. In one embodiment, the gamma irradiation is between about 15 kGy and about 25 kGy. In another embodiment, the gamma irradiation is below 15 kGy, preferably at or below 6 kGy or between about 6 kGy and about 10 kGy.

### Composition of Precursor and Gelling Solution Containing Cryoprotectants

Untreated or pre-treated precursor enzyme-biomaterial and gelling solutions may be mixed with cryoprotectants at different proportions such as hydroxypropyl-β-cyclodextrin, trehalose, polyvinyl pyrrolidone (PVP), and dextran. In one embodiment, the polyvinyl pyrrolidone has an average molecular weight range of >1 kDa to <40 kDa, preferably >1 kDA to <25 kDa, and more preferably has a molecular weight of about 8 kDa. In one embodiment, the dextran has an average molecular weight of 70 kDa.

Tables 1-3 describe the composition of cryoprotectants in the precursor solution (Table 1), the gelling solution (Table 2), and the freeze drying solution (Table 3). The preferred concentration of cryoprotectants for precursor solution and gelling bath is described as % w/v of biomaterial concentration may be from about 0.1% w/v to about 4% w/v and about 0.1% w/v to about 10% w/v for divalent metal ion concentration, respectively. The concentration of trehalose, hydroxypropyl-β-cyclodextrin, PVP, and dextran may range from about 0.1% w/v to about 20% w/v, about 0.1% w/v to about 2% w/v, about 0.1% w/v to about 1% w/v and about 0.1% w/v to about 1% w/v, respectively. Other cryoprotectants such as glucose, lactose, maltodextrins, mannitol, glycols, and polyglycols can also be used Stabilizer 1 in similar proportions to that discussed above. In one embodiment, the glucose, lactose, maltodextrins, mannitol, glycol, or polyglycol Stabilizer 1 is used along with Stabilizer 2.

TABLE 1
Composition of cryoprotectants in precursor solution

| Formulation | Stabilizer 1 | Stabilizer 2 |
|---|---|---|
| 1 | Trehalose (0.1-5% w/v) | Dextran 70 kDa |
| | | (0.1-10% w/v) |

TABLE 2
Composition of cryoprotectants in gelling solution

| Formulation | Stabilizer 1 | Stabilizer 2 |
|---|---|---|
| 1 | Trehalose (0.1-5% w/v) | — |
| 2 | Trehalose (0.1-5% w/v) | Dextran (70 kDa) |
| | | (0.1-10% w/v) |
| 3 | Trehalose (0.1-5% w/v) | PVP (0.1-10% w/v) |

TABLE 3
Composition of cryoprotectants in freeze drying solution

| Formulation | Stabilizer 1 | Stabilizer 2 | Stabilizer 3 |
|---|---|---|---|
| 1 | Trehalose | Dextran 70 kDa | PVP |
| | (0.1-5% w/v) | (0.1-10% w/v) | (0.1-10% w/v) |
| 2 | Hydroxypropyl-β- | Dextran 70 kDa | PVP |
| | cyclodextrin | (0.1-10% w/v) | (0.1-10% w/v) |
| | (0.1-5% w/v) | | |

These cryoprotectant components may be mixed with the untreated and pre-treated precursor solutions for 15 min to 1 hours before adding into gelling bath containing cryoprotectants for crosslinking with divalent metal ion to form enzyme loaded self-degrading biologically derived microspheres containing an enzyme and cryoprotectants. These microspheres may be dehydrated to obtain dried particles with the moisture content of about 10% by mass or less, about 8% by mass or less, about 6% by mass or less, about 4% by mass or less, about 3% by mass or less, preferably about 2% by mass or less, and more preferably about 1% by mass or less. Under dry conditions sealed in a vial, the dried particles may be further subjected to sterilization, such as gamma or e-beam radiation described elsewhere herein. The dehydrated and sterilized enzyme loaded divalent metal ion-crosslinked biologically derived microspheres can be stored for ≤24 months.

Compositions and Methods of Preparing Photo-Crosslinked Self-Degrading Biologically Derived Microspheres Loaded with an Enzyme

### Composition of Precursor Solution

The self-degrading photo-crosslinked biologically derived particles loaded with an enzyme can be prepared using a biomaterial comprising a photo-crosslinkable moiety and a photoinitiator. In one embodiment, the photo-crosslinkable moiety is an acrylate or methacrylate moiety. The degradation of the photo-crosslinked particles can be modulated by controlling the composition of precursor solution which comprises (i) the enzyme (may be pre-treated with pH, temperature, metal ion inhibitors, competitive inhibitors, non-competitive inhibitors, and end-product inhibitors) loaded into photo-crosslinked biologically derived microspheres and (ii) the biomaterial of a predetermined molecular weight that comprises a photo-crosslinkable moiety. In one embodiment, the degradation of the photo-crosslinked particles can also be modulated by varying the ratio of photoinitiator to the biomaterial concentration and varying the duration of photo irradiation affecting the crosslinking of the particles. In embodiments wherein the biomaterial comprising a photo-crosslinkable moiety is alginate, the ratio of M (β-D-mannuronic acid) and G (α-L-guluronic acid) blocks (M/G) may also be used to modulate the degradation of the resulting photo-crosslinked particles.

Similar to divalent metal ion crosslinked biologically derived microspheres, the precursor solution containing the enzyme may degrade the biomaterial. This may reduce the viscosity of the biomaterial, reduce the enzyme loading capacity of the biomaterial, and adversely affect the particle's size and shape. Specifically, in the precursor solution, the enzyme causes breakdown of the biomaterial which can impact the encapsulation of the enzyme into the biologically derived particles. This also reduces the initial viscosity of the solution of biomaterial, which is important for maintaining both encapsulation efficiency within the biologically derived particles and also for obtaining particles of desired size and shape. Therefore, the precursor solution may or may not be pre-treated at different pH, a low temperature, and/or by exposure to a metal ion inhibitor, a competitive inhibitor, a non-competitive inhibitor, or an end-product inhibitor before being subjected to the photo-irradiation. In one embodiment, the biomaterial is alginate and alginate lyase in the precursor solution degrades the alginate.

All of the pre-treatment processes of the precursor solution are the same as those mentioned for the preparation of divalent metal ion-crosslinked microspheres described elsewhere herein.

The optimum catalytic activity of alginate lyase is observed at a pH ranging from 6.8 to 7.5. To prevent the initial degradation of the biomaterial during the preparation of the enzyme loaded biologically derived particles, the pH of the enzyme-biomaterial solution may be reduced to 3.0. To carry out this process, sodium acetate-acetic acid buffer, of ionic strength <1 M, preferably <0.1 M and most preferably <0.01 M with a pH range 3.7-5.6 is used. In addition, the desired pH (pH 6.5 to 3.0) of the solution may also be achieved using sodium hydroxide (>1 M to <0.01 M) or hydrochloric acid (>1 M to <0.01 M). This results in the reduction or ceasing of the enzyme catalytic activity. This regulation of the catalytic activity may be attributed to the unfolding of 3D conformation of enzyme. The ceased catalytic activity of the enzyme may be reversed/activated by exposing the enzyme loaded biologically derived particles to an aqueous environment having a pH of 6.5 to 7.5. The preferred buffers for reversing the activity of the enzyme are phosphate buffers. The preferred ionic strength of the phosphate buffer is 0.01 M with a pH range of 6.5 to 7.5 at 20° C. The desired pH (pH 6.5 to 7.5) of the solution may also be Additionally, saline or de-ionized water or an aqueous solution having a pH between 6.5 to 7.5 may also be used.

In combination with changing the pH of the solution, the temperature of the individual components of the precursor solution before mixing may be maintained at 1-4° C. to inhibit the degradation of biomaterial. The temperature of precursor solution after mixing the individual components may be maintained at 1-4° C. to inhibit the degradation of biomaterial. Note that the temperature will also influence the viscosity of the solution.

In addition to changing the pH and temperature of the precursor solution, the enzyme may be pre-treated with metal ion inhibitors such as Cu²⁺, Zn²⁺, and Fe³⁺. These metal ions can inhibit the activity of the enzyme (Inoue, et al., “Functional identification of alginate lyase from the brown alga Saccharina japonica,” Sci. Rep. 2019; 9:1-11).

In combination with changing pH and temperature, other classes of organic and inorganic inhibitors including competitive, non-competitive, and end-product inhibitors can also be added at different stages of particles synthesis (e.g., in the precursor solution, the gelling solution, or the freeze-drying solution).

### Synthesis of Self-Degrading Photo-Crosslinked Biologically Derived Microspheres

To make the self-degrading photo-crosslinked biologically derived microspheres, water soluble photoinitiators such as Irgacure 2959, Irgacure 184, Irgacure 651, Irgacure 369, and Irgacure 907 may be used. The preferred photoinitiator is Irgacure 2959. These photoinitiators activate and form radicals upon the irradiation with ultraviolet light of 320 nm to 410 nm wavelength, but the ideal wavelength is 365 nm. The degradation of the microspheres also depends on the concentration of biomaterial comprising a photo-crosslinkable group, the concentration of the photoinitiator, and the duration of UV irradiation. The concentration of the biomaterial (e.g., alginate) comprising a photo-crosslinkable group and the photoinitiator in the precursor solution may have a range of 0.1% w/v to 4% w/v and 0.1% w/v to 1.5% w/v, respectively. For the fast-degrading enzyme loaded biologically derived microspheres (>20 minutes to <4 hours), the preferable concentrations of the biomaterial and photoinitiator may be 1% w/v to 1.5% w/v and 0.1% w/v to 0.3% w/v, respectively. For the intermediate degradation period microspheres (5 days to 30 days), the preferable concentrations of the biomaterial and photoinitiator may be 2% w/v to 3% w/v and 0.4% w/v to 0.8% w/v, respectively. For slow degrading enzyme loaded biologically derived particles, the preferable concentrations of the biomaterial and photoinitiator may be 3% w/v to 4% w/v and 0.9% w/v to 1.5% w/v, respectively. Furthermore, the duration of UV irradiation may range from >10 seconds to <10 minutes, wherein the preferable duration for the fast-degrading microspheres may be <1 minute, 1-5 minutes for intermediately degrading microspheres, and >5 mins but <10 mins for slow degrading microspheres.

In conjunction with the concentration of photoinitator and duration of UV irradiation, the degradation of alginate-alginate lyase microspheres is also dependent on G/M ratio and viscosity of purified alginate. The percentage of M (β-D-mannuronic acid) content in the purified alginate may be 50%-80%, 55%-75%, or 60%-80%. The preferred M content may be in the range of 55% to 65% to obtain particle degradation in a shorter and intermediatory time period. To get intermediate (5 days to 30 days) or slow (>30 days) degradation periods, the higher G content alginate (e.g., the lower M:G ratio) having high molecular weight/viscosity may be used. In certain embodiments, the purified alginate contains more than 50% G content (α-L-guluronic acid). The percentage of G content in the purified alginate may be 50%-80%, 55%-75% and 60%-80%. The viscosity of fast degrading alginate microspheres (>20 minutes to <4 hours) have low molecular weight/low viscosity (<70 mPas to >5 mPas). To achieve intermediate (5 days to 30 days) or slow (>30 days) degradation periods, a biomaterial having high molecular weight/high viscosity may be used which can be considered >70 mPas.

Methods to prepare the microspheres may include a drop-casting technique wherein droplets of precursor solution may be printed on a super-hydrophobic surface (e.g., PTFE), or generated using, for example, single or double emulsion-microfluidics platforms. Upon generation of droplets of the desired size, UV light may be used to crosslink the biomaterial to form spherical enzyme loaded biologically derived microspheres.

In one embodiment, methods to prepare the microspheres may include an electro-encapsulation process. In one embodiment, the electro-encapsulation process comprises passing a precursor solution through a needle with an electrostatic potential to form droplets. In one embodiment, the precursor solution comprises an enzyme and a biomaterial described elsewhere herein. Upon generation of droplets of the desired size, a divalent metal ion may be used to crosslink the biomaterial to form enzyme loaded biologically derived microspheres. In some embodiments, the electrostatic potential is between about 0.1 kV and about 20 kV, about 0.1 kV and about 18 kV, about 0.1 kV and about 16 kV, about 0.1 kV and about 14 kV, about 0.1 kV and about 12 kV, about 0.1 kV and about 10 kV, about 0.1 kV and about 8 kV, about 0.1 kV and about 6 kV, about 1 kV and about 6 kV, about 1 kV and 4 kV, about 2 kV and 4 kV, or about 3 kV.

Use of Bioactive Agents Encapsulated within the Biologically Derived Particles

In one embodiment, the enzyme loaded self-degrading biologically derived particles of the present disclosure can encapsulate an anti-inflammatory agent or an antioxidant, which functions to provide localized pain relief when administered to a subject. In one embodiment, the enzyme loaded self-degrading biologically derived particles of the present disclosure can encapsulate a chemotherapeutic agent. Previous reports demonstrated that high molecular weight hyaluronic acid (100-500 kDa) or its breakdown products display anti-inflammatory and immunosuppressive activity. Therefore, the use of hyaluronic acid encapsulated within the enzyme loaded biologically derived particles of the present invention could provide pain relief when administered to a subject. Similarly, other bioactive agents, including anti-inflammatory agents, NSAIDs, non-NSAIDs, corticosteroids, and antioxidants would also be expected to provide pain relief when administered to the subject. Exemplary anti-inflammatory agents, NSAIDs, non-NSAIDs, corticosteroids, and antioxidants are described elsewhere herein.

To the precursor solution mentioned previously mentioned, bioactive agents such as high molecular weight hyaluronic acid may be added. This involves the addition of the bioactive agent at 1% wt to 20% wt of biomaterial concentration in the precursor solution. On crosslinking in the divalent metal ion-gelling bath, the bioactive agent may be encapsulated. In one embodiment, high molecular weight hyaluronic acid is encapsulated in the enzyme loaded-divalent metal ion crosslinked biologically derived microspheres described elsewhere herein.

In one embodiment, the bioactive agent provides localized pain relief in a subject suffering from tendinopathy. In another embodiment, the bioactive agent provides localized pain relief in a subject during an embolization medical intervention. The encapsulated bioactive agent may be released at the site of embolization or the site of tendinopathy or osteoarthritis due to the degradation of enzyme loaded biologically derived microspheres. For instance, many embolization medical interventions cause neuropathic pain which may be relieved by the use of hyaluronic acid or its breakdown products, alginate and its breakdown products, NSAIDs, non-NSAIDs, corticosteroids, or antioxidants. These pharmacologically active molecules may alleviate pain arises due to tendinopathy and osteoarthritis.

In another embodiment, the self-degrading biologically derived particles of the present disclosure comprise a carbohydrate particle which encapsulates an enzyme that can act on the carbohydrate, wherein the particle does not encapsulate an anti-inflammatory agent or antioxidant. In one embodiment, the enzyme breaks down the carbohydrate particle, forming oligosaccharides which have an anti-inflammatory effect. Therefore, in some embodiments, the particles of the disclosure are able to provide an anti-inflammatory effect and/or pain relief when administered to a subject even when the particles of the disclosure do not encapsulate an anti-inflammatory agent or antioxidant.

### Method of Making Biologically Derived Particles and Enzyme Encapsulated Biologically Derived Particles Using Microfluidics

The enzyme loaded biologically derived particles described herein can be produced through droplet-microfluidics which provides precise control on the shape, size, and morphology of the resulting biomaterial droplets. Typically, a biomaterial solution is mixed with water soluble Ca-EDTA or water insoluble CaCO₃ particles and emulsified in the oil phase on a microfluidics platform to generate the droplets of biomaterial solution having a desired size and shape. The droplets of biomaterial can be crosslinked to a bivalent Ca²⁺ ion released from Ca-EDTA or CaCO₃ under acidic conditions. This crosslinking can be done through “on-chip” or “off-chip” methods to generate Ca²⁺-crosslinked biologically derived particles.

However, this conventional method is difficult to use since the enzyme can be encapsulated in the biologically derived particles effectively in an acidic pH environment. Therefore, if the above conventional method is used to encapsulate the enzyme in a biologically derived particle, the enzyme-biomaterial mixture containing Ca-EDTA or CaCO₃ under acidic conditions would become gelled and the solution could not be passed through the microfluidics platform to generate biomaterial precursor solution droplets. To overcome this drawback, a new method is presented below to successfully generate the enzyme encapsulated biologically derived particles using microfluidics.

The encapsulation of enzyme into biologically derived particles of the present disclosure is performed using a droplet microfluidics method wherein a precursor solution is prepared in a buffer of pH 10 at a temperature of 1-4° C., and wherein the precursor solution comprises a biomaterial, an enzyme, and Ca-EDTA or CaCO₃. In one embodiment, the precursor solution comprises an excipient. In another embodiment, an excipient is used in a solution to wash the enzyme encapsulated biologically derived particles formed from the precursor solution. In one embodiment, water comprising an excipient is used to wash the enzyme encapsulated biologically derived particles formed from the precursor solution. The biomaterial can have a predetermined molecular weight. In embodiments wherein the biomaterial is alginate, the alginate can have a predetermined G/M ratio. The concentration of Ca-EDTA or CaCO₃ is in the range of 1 M to 0.01 M and the preferable concentration is 0.05 M to 0.1 M.

The activity of the alginate lyase enzyme may range from 0.025 U/mg to 1 U/mg of alginate. For the rapid degradation of alginate microspheres >20 minutes to <4 hours, the preferred activity of the enzyme may range from 0.0075 U/mg to 0.25 U/mg of alginate. To get intermediate (5 days to 30 days) or slow (>30 days) degradation periods, the preferred range of enzyme activity may be <0.005 U/mg to ≥0.0025 U/mg of alginate and <0.0025 U/mg of alginate respectively.

The buffer solution has a pH range of 8.0-13.0, and the preferred range of buffer is 9.0-11.0. The buffer solution can be any buffer solution known to a person of skill in the art. Common buffers that can be used to prepare the biomaterial, enzyme, and Ca-EDTA/CaCO₃ solution are disodium hydrogen phthalate/sodium dihydrogen orthophosphate, barbitone sodium/hydrochloric acid, dipotassium hydrogen phthalate/potassium dihydrogen orthophosphate, potassium dihydrogen orthophosphate/sodium hydroxide, barbitone sodium/hydrochloric acid, tris(hydroxylmethyl)aminomethane/hydrochloric acid, sodium tetraborate/hydrochloric acid, glycine/sodium hydroxide, sodium carbonate/sodium hydrogen carbonate, sodium tetraborate/sodium hydroxide, sodium bicarbonate/sodium hydroxide, sodium hydrogen orthophosphate/sodium hydroxide and potassium chloride/sodium hydroxide. The most preferred buffer system is sodium bicarbonate/sodium hydroxide. The ionic strength of the buffer is <1 M, and the preferred range is <0.5 M to >0.05 M, and most preferably ≤0.1 M. The preferred temperature range is <10° C. and most preferably >1° C. to <4° C.

The above conditions allow the generation of biomaterial droplets and overcome the challenges that are encountered with the conventional method to prepare enzyme encapsulated biologically derived particles in two ways—(a) these conditions inhibit the activity of the enzyme thus preventing the initial degradation of biomaterial in the precursor solution and (b) these conditions prevent the release of the Ca²⁺ ion from Ca-EDTA or CaCO₃. Furthermore, the precursor solution along with oil passes through a suitable microfluidics chip to form biomaterial droplets (water-in-oil emulsion method). These droplets are crosslinked by a bivalent Ca²⁺ ion by exposing them to acetic acid of concentration 0.01% v/v to 5% v/v. The preferred range of acetic acid concentration is 1% to 2% v/v of acetic acid. On exposure to acetic acid, the Ca²⁺ ion gets released from Ca-EDTA or CaCO₃ and binds with egg-boxes of the biomaterial to form Ca²⁺⁻ crosslinked enzyme loaded biologically derived particles. These particles are then washed with deionized water containing excipients to remove the acid. If required, the particles are further crosslinked in a calcium chloride solution of concentration ranging from 2% w/v to 10% w/v. The washed particles are suspended in a solution containing excipients for the duration of 6-24 hours and dried. The dried particles can be reconstituted in a neutral pH buffer to activate the enzyme and initiate the degradation of the enzyme loaded biologically derived particles.

### Water Content and Sterilization of Biologically Derived Particles

The shelf-life of the self-degrading enzyme loaded biologically derived microspheres loaded with an enzyme may be improved through dehydrating the microspheres such that they are substantially free of water and sterilizing the dehydrated microspheres.

The microspheres can be dehydrated using any technique known to a person of skill in the art. In one embodiment, the microspheres are lyophilized. In another embodiment, the microspheres are dehydrated using super-critical CO₂. In one embodiment, the microspheres are dried such that the water content of each microsphere is less than about 15%, less than about 12%, less than about 10%, less than about 8%, less than about 6%, less than about 4%, or less than about 1% by mass. In one embodiment, the water content of each microsphere is between about 1% and about 10% by mass. Although not wishing to be limited by theory, it is believed if the water content of the microsphere is too high, the enzyme is denatured by the sterilization while if the water content is too low, the microspheres do not hydrate properly and also the enzyme activity is also affected (a little water is needed to maintain the enzyme 3D conformation and activity).

For these post-preparation processes, cryoprotectants may be added into precursor solution (described elsewhere herein). The addition of cryoprotectants is important in many ways. Firstly, it helps in maintaining the sphericity and mechanical robustness of the enzyme loaded biologically derived particles during the dehydration process. Secondly, it preserves the 3D conformation of the enzyme at extremely low temperatures and freezing cycles used during lyophilization, thereby preserving the enzyme activity. Recovery of microsphere shape post lyophilization was considered as an issue. This is not unexpected, as high-water content gels shrink during lyophilization and often do not re-establish their original shape upon rehydration. The addition of cryoprotectants such as sugars and polymers can compensate for this, to help maintain the porous structure during sublimation of the internal water within the structure. Also, it has been observed that the residual activity of the enzyme reduced significantly when the lyophilization of the enzymes was performed without the addition of the cryoprotectants/cryopreservation medium. Thus, the use of cryoprotectants also assists the rapid recovery of shape upon reconstitution in aqueous medium and enables retention of the functionality of the active ingredients including enzymes.

The microspheres can be sterilized using any technique known to a person of skill in the art. In one embodiment, the microspheres are sterilized using irradiation. In one embodiment, the irradiation is e-beam irradiation. In another embodiment, the irradiation is gamma irradiation. In one embodiment, the gamma irradiation is between about 15 kGy and about 25 kGy. In another embodiment, the gamma irradiation is below 15 kGy, preferably at or below 6 kGy.

### Composition of Precursor Solution Containing Cryoprotectants

Untreated or pre-treated precursor enzyme-biomaterial solutions (as described elsewhere herein) may be mixed with cryoprotectants, such as hydroxypropyl-β-cyclodextrin, trehalose, polyvinyl pyrrolidone, and dextran, at different proportions. In one embodiment, the polyvinyl pyrrolidone has a molecular weight of 40 kDa (PVP 40 kDa). In one embodiment, the polyvinyl pyrrolidone has an average molecular weight range of >1 kDa to <40 kDa, preferably >1 kDA to <25 kDa, and more preferably has a molecular weight of about 8 kDa. In one embodiment, the dextran has a molecular weight of 70 kDa.

Tables 1-3 describe the composition of cryoprotectants in the precursor solution (Table 1), the gelling solution (Table 2), and the freeze-drying solution (Table 3). The preferred concentration of cryoprotectants for precursor solution is described as % w/v of biomaterial concentration. The concentration of trehalose hydroxypropyl-β-cyclodextrin, PVP, and dextran may range from about 0.1% w/v to about 5% w/v, about 0.1% w/v to about 5% w/v, about 0.1% w/v to about 10% w/v, and about 0.1% w/v to about 10% w/v, respectively.

These cryoprotectant components may be mixed with the untreated and pre-treated precursor solutions for 15 minutes to 3 hours. The precursor solution may contain a photoinitiator or the photoininator may be added after the precursor solution sits for 15 minutes to 3 hours. In one embodiment, the precursor solution comprising the cryoprotectant and photoiniator is then irradiated as described elsewhere herein, forming photo-crosslinked self-degrading biologically derived microspheres which encapsulate the enzyme and cryoprotectant. These microspheres may be dehydrated to obtain dried particles with the moisture content of about 10% by mass or less, about 8% by mass or less, about 6% by mass or less, about 4% by mass or less, about 3% by mass or less, preferably about 2% by mass or less, and more preferably about 1% by mass or less. Under dry conditions sealed in a vial, the dried particles may be further subjected to sterilization, such as gamma or e-beam radiation described elsewhere herein. The dehydrated and sterilized enzyme loaded divalent metal ion-crosslinked biologically derived microspheres can be stored for ≤24 months.

### Additional Embodiments

The concentration of the biomaterial can also affect the pore size and robustness of the divalent complexed enzyme loaded biologically derived particle. The concentration of the biomaterial can be about 0.05% weight by volume (w/v), 0.10% w/v, 0.15% w/v, 0.20% w/v, 0.25% w/v, 0.30% w/v, 0.35% w/v, 0.40% w/v, 0.45% w/v, 0.50% w/v, 0.60% w/v, 0.70% w/v, 0.80% w/v, 0.90% w/v, 1.0% w/v, 1.25% w/v, 1.5% w/v, 1.75% w/v, 2.0% w/v, 2.25% w/v, 2.5% w/v, 2.75% w/v, 3.0% w/v, 3.25% w/v, 3.5% w/v, 3.75% w/v, 4% w/v, 4.25% w/v, 4.5% w/v, 4.75% w/v, 5.0% w/v, 5.25% w/v, 5.5% w/v, 5.75% w/v, 6.0% w/v, or greater than about 6.0% w/v for the preparation of rapid >20 minutes to <4 hours) and slow degrading (5 days to 30 days) enzyme loaded divalent metal ion complexed biologically derived particles.

In addition, the gelling time during which the enzyme loaded biologically derived particles are crosslinked within the metal ion bath can also affect the size, sphericity, and physical robustness of the divalent metal ion complexed biologically derived particles. Generally, the term “sphericity” can refer to a measure of how closely the shape of an object resembles that of a perfect sphere. The roundness of an injectable substance can be important, for example, as abnormally shaped substances can have difficulty in travelling through blood vessels, leading to clogged blood vessels, thereby blocking blood flow to various parts of the body The gelling time can be less than about 1 min, less than about 2 minutes, less than about 3 minutes, less than about 4 minutes, less than about 5 minutes, less than about 6 minutes, less than about 7 minutes less than about 8 minutes, less than about 9 minutes, less than about 10 minutes, less than about 11 minutes, less than about 12 minutes, less than about 13 minutes, less than about 14 minutes, less than about 15 minutes, less than about 20 minutes, less than about 25 minutes, or less than about 30 minutes.

To achieve the desired degradation period of the enzyme loaded biologically derived particles, the amount of enzyme mixed with the biomaterial may be varied. The amount of enzyme mixed with the biomaterial varies from <1 unit to 50 units/mL of biomaterial for the preparation of rapid (>20 minutes to <4 hours) intermediate (5 days to 30 days), or slow (>30 days) degrading divalent metal ion complexed biologically derived particles. For the sake of clarity, in enzymology 1 unit (U) is the amount of enzyme that catalyzes the reaction of 1 μmol of substrate per minute. The amount of enzyme loading into the divalent metal ion complexed biologically derived particles also depends on the molecular weight or viscosity of the biomaterial.

Furthermore, the degradation of enzyme-loaded biologically derived particles could also be controlled by regulating the enzyme activity. In order to control the catalytic degradation activity of the biologically derived particles, the enzyme may be complexed or pre-treated with <1 mM of Cu²⁺, Zn²⁺, and Fe³⁺ metal ions. These metal ions can inhibit enzymatic activity by approximately 90%. Other metal ions, such as Mg²⁺ and Ca²⁺ at 1 mM concentration, reduces the activity by 20% to 50%, respectively. The free or unbound metal ions may be removed from the solution through dialysis. These metal ions can inhibit the activity of the enzyme and can be considered detrimental for the enzyme (Inoue, et al., “Functional identification of alginate lyase from the brown alga Saccharina japonica,” Sci. Rep. 2019; 9:1-11). On the contrary, the same concept is adopted in certain embodiments of the present disclosure to regulate the degradation of enzyme loaded biologically derived particles. Under in vivo conditions, the enzymatic activity could be regulated solely using these metal ions to achieve the rapid (>20 minutes to <4 hours) and longer (5 days to 30 days or >30 days) duration degrading particles.

In general, an enzyme may be immobilized into an inert or insoluble matrix. This provides resistance to physiological factors affecting the enzymatic reactions such as pH or temperature and also increase the rate of reaction. It also keeps the enzyme localized in a place (e.g, inside the particles, surface decorated, etc.). In certain embodiments of the present disclosure, the modified or native enzyme is immobilized/encapsulated in a reactive biomaterial substrate (instead of an inert matrix). Therefore, another important aspect is to avoid the initial degradation during the manufacturing of the enzyme loaded biologically derived particles from the enzyme-biomaterial precursor solution. To overcome this problem, the following approaches are proposed for use in certain embodiments of the present disclosure.

The enzyme may be pre-treated with the metal ions inhibitors such as Cu²⁺, Zn²⁺, Fe³⁺, Mg²⁺, and Ca²⁺. These metal ions, at optimum concentrations without affecting the physical robustness of the particles, may reduce the degradation of the particles by partially inhibiting the enzyme activity.

Another approach is to reduce the temperature of the enzyme-biomaterial precursor solution from ambient to a temperature ranging from 4 to 10° C. This will reduce or cease the catalytic activity of the enzyme, thereby preventing the degradation of biomaterial. In addition, the temperature of the divalent metal ions gelling bath may also be reduced to the range of 1 to 10° C. This metal ion bath is used for gelling the droplets of enzyme-biomaterial solution to form the divalent metal ions-complexed enzyme loaded biologically derived particles.

The catalytic activity of the enzyme may also be regulated by changing the pH of the enzyme-biomaterial and gelling bath solutions. The catalytic activity of the alginate lyase enzyme used in the present disclosure is optimally at a pH ranging from 6.8 to 7.5 (see, e.g., Farrés, et al., “Formation kinetics and rheology of alginate fluid gels produced by in-situ calcium release,” Food Hydrocolloids 40 (2014): 76-84). To prevent the initial degradation of the biomaterial during the preparation of enzyme loaded biologically derived particles, the pH of the enzyme-biomaterial solution may be reduced to 3.0. To carry out this process, a buffer having an ionic strength <1 M, preferably <0.1 M, and most preferably <0.01 M, with a pH range of 3.7 to 5.6 is used. In one embodiment, the buffer is a sodium acetate-acetic acid buffer. In addition, the desired pH (pH of 6.5 to 3.0) of the solution may also be achieved using sodium hydroxide (>1 M to <0.01 M) or hydrochloric acid (>1 M to <0.01 M). This results in the reduction or ceasing of the enzyme catalytic activity. This regulation of the catalytic activity may be attributed to the unfolding of 3D conformation of the enzyme. The ceased catalytic activity of the enzyme may be reversed/activated by exposing the enzyme loaded biologically derived particles to an aqueous environment having a pH of 6.5 to 7.5. The preferred buffer to reverse the activity of the enzymes of the present disclosure is a phosphate buffer. The preferred ionic strength of the phosphate buffer is 0.01 M with a pH range of 6.5 to 7.5 at 20° C. The desired pH (pH of 6.5 to 7.5) of the solution may also be achieved using sodium hydroxide (>1 M to <0.01 M) or hydrochloric acid (>1 M to <0.01 M). Additionally, saline, deionized water, or an aqueous solution having a pH between 6.5-7.5 may also be used.

Therefore, a combination of the abovementioned approaches may be used efficiently to encapsulate or load the enzyme into the biologically derived particles complexed/gelled with divalent metal ions without degrading the biologically derived matrix.

The precursor enzyme-biomaterial solution under the appropriate conditions (low temperature and pH) requires gelling in a divalent metal ion bath containing one or more cryoprotectants in order to crosslink the resulting enzyme loaded biologically derived particles. The composition and condition of the gelling bath are important to make desired enzyme loaded biologically derived particles. The divalent metal ion component of the gelling bath composition may be selected from the group consisting Cu²⁺, Ba²⁺, Sr²⁺, Ca²⁺, Co²⁺, Ni²⁺, Mn²⁺, and Mg²⁺ (Lee, et al., “Alginate: properties and biomedical applications,” Progress in polymer science 37, no. 1 (2012): 106-126; and Brus, et al., “Structure and dynamics of alginate gels cross-linked by polyvalent ions probed via solid state NMR spectroscopy,” Biomacromolecules 18, no. 8 (2017): 2478-2488). Divalent cation choice may also influence the crosslinking of the biologically derived particles. The binding strength of the divalent metal ion with alginate is given in decreasing order Cu²⁺>Ba²⁺>Sr²⁺>Ca²⁺>Co²⁺>Ni²⁺>Mn²⁺>Mg²⁺, wherein the preferred metal cations for alginate are Ba²⁺ and Ca²⁺. These metal ions may be used at different concentrations ranging from 0.1% w/v to 10% w/v. The addition of cryoprotectants in the gelling bath is important in four ways: (a) it helps in maintaining the sphericity and mechanical robustness of the enzyme loaded biologically derived particles during dehydration of the particles, (b) it also preserves the 3D conformation of the enzyme in extremely low temperatures and freezing cycles used during lyophilization, thereby preserving the enzyme activity when this technique is used for dehydration, (c) it prevents the formation large ice crystal and helps in maintaining the matrix of microspheres as well as cake (contains cryoprotectants and microspheres) during freeze drying process, and (d) it reduces the reconstitution of the lyophilized product in aqueous medium without creating effervescence or trapped air bubbles.

In many instances, it has been observed that the residual activity of the enzyme is reduced significantly when lyophilization of the enzyme was performed without the addition of the cryoprotectants/cryopreservation medium (Tamiya, et al., “Freeze denaturation of enzymes and its prevention with additives,” Cryobiology 22, no. 5 (1985): 446-456; and Porter, et al., “Effects of freezing on particulate enzymes of rat liver,” J. Biol. Chem 205 (1953): 883-891). The cryoprotectant components may include those known in the art, such as sucrose, glycerol, ethylene glycol, sorbitol, trehalose, propylene glycol, or proprietary/commercially available cryoprotectants. When these cryoprotectants are added into the gelling bath, they get encapsulated or uniformly distributed in the matrix of the biologically derived particles (Chan, et al., “Effects of starch filler on the physical properties of lyophilized calcium-alginate beads and the viability of encapsulated cells,” Carbohydrate polymers 83, no. 1 (2011): 225-232).

Additionally, a cryoprotectant may also be used in the post-processing stage instead of adding the cryoprotectant during the manufacturing process of these particles in the gelling bath containing-divalent metal ions. For example, when freeze-dried enzyme loaded biologically derived particles are prepared, the cryoprotectant can be used at this post-processing step. In this process, the droplets of the precursor enzyme-biomaterial solution are added into the gelling bath containing divalent metal ion only to form enzyme loaded biologically derived particles. Following the isolation of these particles from the gelling bath, they may be soaked in a suitable cryoprotectant and subjected to the freeze-drying process. Under freeze-drying conditions, the cryoprotectant prevents freeze denaturation of the enzyme as well as providing the defect-free enzyme loaded biologically derived particles by preventing the collapse of the gel structure by filling the pores formed as the water is sublimed out of the matrix. The particle size may be >40 μm, <200 μm, but <2000 μm. Also, this process may be used to prepare enzyme loaded biologically derived particles of different morphologies such as microfibrils, core-shell particles, Janus particles, or capsules.

Furthermore, in certain embodiments, the present disclosure provides the preparation of enzyme loaded biologically derived particles that are further loaded with radiopaque and/or a drug. To achieve this, a composition comprising Ca²⁺ ions and an X-ray contrasting metal ion such as barium, gadolinium, and tantalum metal ions (Yu, et al., “Metal-based X-ray contrast media,” Chemical Reviews 99, no. 9 (1999): 2353-2378) is proposed to be used in the gelling bath. Another proposed approach is the reconstitution of the enzyme loaded biologically derived particles with commercially available radiopaque agents, which become temporarily absorbed into the matrix as the biologically derived matrix swells in the aqueous medium. The proposed method of loading the drugs/bioactive agents (anticancer and osteogenic) into the enzyme loaded biologically derived particles involves exposing these particles to the drug for 2 to 3 hours. The delivery of the drug in the body will be facilitated by the in situ degradation mechanism of enzyme loaded biologically derived particles.

In certain embodiments, the enzyme loaded biologically derived microspheres may be stored over extended periods of time. In certain embodiments, metal ion complexed-enzyme is immobilized into its substrate. It is contemplated that the slow degradation of the matrix starts during storage. This degradation may be stopped by suspending the microspheres in a pH below 5.5. Apart from reducing the operating temperature below 10° C. to prevent the degradation of alginate microspheres, an alternative method is to freeze or vacuum dry the microspheres. In certain embodiments, the dried microspheres may be loaded into a specially designed syringe.

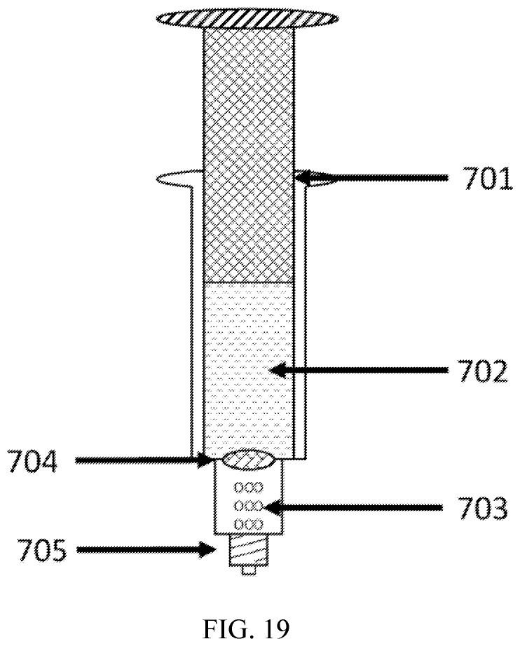

FIG. 19 illustrates a proposed design of a syringe for reconstituting and/or administering microspheres of the present disclosure. The syringe may comprise a plunger 701, which may be in a locked or unlocked position. The syringe may also comprise a first chamber comprising a suspension medium 702 and a second chamber comprising dried microspheres 703. Generally, the syringe may be constructed and arranged such that the contents of each of the chambers of the syringe are separated (e.g., fluidically) until pressure is applied to the plunger, thereby mixing the contents of each chamber (e.g., reconstituting the microspheres). It is contemplated that any multi-chamber lyophilization syringe known in the art may be used. In certain embodiments, the syringe may comprise a breakable membrane 704 separating the first chamber 701 and the second chamber 702. When pressure is applied to the plunger 701, the breakable membrane is broken and the contents of the first chamber comes into contact with the dried microspheres of the second chamber 703 to reconstitute the microspheres. In other embodiments, the syringe comprises a liquid bypass duct. In yet another embodiment, a barrier separating the first chamber and the second chamber of the syringe can comprise a one way valve. When pressure is applied to the plunger 701, the one way valve is forced open and the contents of the first chamber comes into contact with the dried microspheres of the second chamber to reconstitute the microspheres. In some embodiments, the reconstitution medium may be water for injection (WFI), saline, or a buffer with a pH in an acidic, alkaline, or neutral range. Once reconstituted, the microspheres are now ready for use. The syringe can comprise a quick connector 705 (e.g., Luer Lock connector) for connecting tubing or the like to administer the reconstituted microspheres to the subject.

### Subjects

A patient treated by any of the methods or compositions described herein may be of any age and may be an adult, infant, or child. In some cases, the patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between 2 and 20 years old, between 20 and 40 years old, or between 40 and 90 years old). The patient may be a human or non-human subject.

Any of the compositions disclosed herein may be administered to a non-human subject, such as a laboratory or farm animal. Non-limiting examples of a non-human subject include laboratory or research animals, a dog, a goat, a guinea pig, a hamster, a mouse, a pig, a non-human primate (e.g., a gorilla, an ape, an orangutan, a lemur, or a baboon), a rat, a sheep, or a cow.

### Additives and Excipients

In some cases, the enzyme loaded biologically derived particles or microspheres described herein may comprise an excipient that may provide long term preservation, bulk up a formulation that contains potent active ingredients, facilitate drug absorption, reduce viscosity, or enhance the solubility of the biologically derived particle or microsphere. An enzyme loaded biologically derived particle or microsphere of the present disclosure may comprise about 1%, 2%, 3%, 4%, 5%, 6%, 7%, 8%, 9%, 10%, 15%, 20%, 25%, 30%, 35%, 40%, 45%, 50%, or greater than about 50% of the excipient by weight or by volume.

In certain embodiments, the enzyme loaded biologically derived particle or microsphere of the present disclosure may comprise one or more solubilizers. As used herein, “solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide, and the like. An enzyme loaded biologically derived particle or microsphere of the present disclosure may comprise about 1%, 2%, 3%, 4%, 5%, 6%, 7%, 8%, 9%, 10%, 15%, 20%, 25%, 30%, 35%, 40%, 45%, 50%, or greater than about 50% of the solubilizer by weight or by volume.

In some embodiments, the compositions described herein include other medicinal or pharmaceutical agents, carriers, adjuvants, such as preserving, stabilizing, wetting or emulsifying agents, solution promoters, and salts for regulating the osmotic pressure, osmolarity, and/or osmolality of the enzyme loaded biologically derived particle or microsphere. In some embodiments, the compositions comprise a stabilizing agent. In some embodiments, stabilizing agent is selected from, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used.

In some embodiments, the composition comprises a suspending agent. Useful suspending agents include for example only, compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol may have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone, and the like.

In some embodiments, the composition comprises an additional surfactant (co-surfactant) and/or buffering agent and/or solvent. In some embodiments, the surfactant and/or buffering agent and/or solvent is a) a natural and synthetic lipophilic agent, e.g., phospholipids, cholesterol, and cholesterol fatty acid esters and derivatives thereof; b) a nonionic surfactant, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens), sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors), sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxyethyleneglycerol triiricinoleate, and any combinations or mixtures thereof; c) an anionic surfactant including, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) a cationic surfactant such as quaternary ammonium compounds, benzalkonium chloride, cetyltrimethylammonium bromide, and lauryldimethylbenzyl-ammonium chloride. It is contemplated that the solvent may be chosen with the intended subject in mind.

In some embodiments, the compositions disclosed herein comprise preservatives. Suitable preservatives for use in the compositions described herein include, but are not limited to benzoic acid, boric acid, p-hydroxybenzoates, phenols, chlorinated phenolic compounds, alcohols, quaternary compounds, quaternary ammonium compounds (e.g., benzalkonium chloride, cetyltrimethylammonium bromide or cetylpyridinium chloride), stabilized chlorine dioxide, mercurials (e.g., merfen or thiomersal), or mixtures thereof.

### Methods of Using the Enzyme Loaded Biologically Derived Particles

In another aspect, the present disclosure provides a method of inducing a self-degrading embolism in a subject in need thereof, comprising administering a plurality of the microspheres described elsewhere herein into a blood vessel of the subject. In one embodiment, the blood vessel is a geniculate artery. In one embodiment, the method induces a prostate arterial embolism, a uterine artery embolism, or, when the enzyme loaded biologically derived particles are mixed with or contain chemotherapeutic agents, induces a transarterial chemoembolism (TACE).

In yet another aspect, the present disclosure provides a method of treating a disease or disorder in a subject in need thereof, comprising administering to the subject a plurality of the microspheres described elsewhere herein. In one embodiment, the microspheres are injected into the subject at the site of the disease or disorder. In one embodiment, the microspheres are injected into the subject at the site of the pain or discomfort from the disease or disorder. In one embodiment, the disease or disorder is tendinopathy. In one embodiment, the disease or disorder is selected from osteoarthritis, frozen shoulder, tennis elbow (lateral epicondylitis), golfer's elbow (medial epicondylitis), pitcher's elbow (flexor tendinitis), Achilles tendinopathy, plantar fasciitis, symptomatic accessory navicular bone, hamstring tendinopathy, jumper's knee (patellar tendonitis), runner's knee (patellofemoral pain syndrome (PFPS)), pes anserine bursitis (knee pain), posterior tibial muscle tendinopathy, wrist (TFCC-Triangular FibroCartilage Complex) tendinopathy, trigger finger (stenosing flexor tenosynovitis), and haemarthrosis.

In another aspect, the present disclosure provides a method of rapidly degrading a microsphere in a subject, comprising administering to the subject a bail out solution, wherein a plurality of microspheres described elsewhere herein was previously administered to the subject and the bail out solution comprises an enzyme, an anion, a phosphate buffer, or a combination thereof. In one embodiment, the enzyme is capable of degrading the microspheres and/or wherein the anion degrades the microspheres by chelating one or more metals in the microspheres. In one embodiment, the subject was previously administered microspheres disclosed herein comprising alginate particles encapsulating alginate lyase and the bail out solution comprises alginate lyase, optionally in combination with a divalent metal chelator. In another embodiment, the subject was previously administered microspheres disclosed herein comprising alginate particles encapsulating alginate lyase and the bail out solution comprises citrate.

In yet another aspect, the present disclosure provides a method of rapidly degrading a microsphere in a subject, comprising administering to the subject a bail out solution, wherein a plurality of microspheres comprising a crosslinked biomaterial and lacking an enzyme was previously administered to the subject. In one embodiment, the microspheres comprising a biomaterial are crosslinked with a divalent metal ion. Exemplary divalent metal ions are described elsewhere herein. In one embodiment, the bail out solution comprises an enzyme capable of degrading the biomaterial and an anion or chelating agent. In one embodiment, the enzyme is complementary to the biomaterial used to make the microspheres. In one embodiment, the microsphere comprises alginate particles and the enzyme is alginate lyase, the microsphere comprises pectin particles and the enzyme is pectinase, the microsphere comprises hyaluronic acid particles and the enzyme is hyaluronidase, the microsphere comprises gelatin particles and the enzyme is a matrix metalloproteinase or protease, the microsphere comprises albumin particles and the enzyme is peptidase, the microsphere comprises collagen particles and the enzyme is protease, the microsphere comprises fibrinogen particles and the enzyme is plasmin, the microsphere comprises silk fibrin particles and the enzyme is protease, the microsphere comprises starch particles and the enzyme is amylase, the microsphere comprises chitosan particles and the enzyme is chitosanase or lysozyme, the microsphere comprises agar/agarose particles and the enzyme is agarase, the microsphere comprises carrageenan particles and the enzyme is carrageenase, the microsphere comprises pullulan particles and the enzyme is pullulanase, the microsphere comprises dextran particles and the enzyme is dextranase, the microsphere comprises b-glycan particles and the enzyme is b-glycanase, the microsphere comprises cellulose particles and the enzyme is cellulase, or the microsphere comprises lignin particles and the enzyme is ligninase.

### Kits

In another aspect, the present disclosure provides a kit.

In one embodiment, the kit comprises enzyme loaded biologically derived particles described elsewhere herein. In one embodiment, the particles are microspheres. In one embodiment, the enzyme loaded biologically derived particles are photo-crosslinked. In another embodiment, the enzyme loaded biologically derived particles are crosslinked using a divalent metal ion, a heterobifunctional crosslinker, or a homobifunctional crosslinker. In yet another embodiment, the enzyme loaded biologically derived particles are gelled using a thermogelation method. In one embodiment, the kit comprises a solvent in which to reconstitute, dissolve, suspend, or disperse the particles or microspheres of the disclosure. In another embodiment, the kit does not comprise enzyme loaded biologically derived particles.

In one embodiment, the kit comprises one or more components to form a “bail out” solution which provides for rapid degradation of the particles or microspheres of the disclosure in an emergency setting. In one embodiment, one component to form the bail out solution comprises an enzyme that can act on the particles or microspheres of the disclosure to degrade them. In one embodiment, the enzyme is a complementary enzyme to the biomaterial used to make the biologically derived particles. Therefore, in embodiments wherein alginate is used as a biomaterial to make the biologically derived particles, the enzyme used to form the bail out solution is an alginate lyase. In embodiments wherein one component to form the bail out solution comprises an enzyme, the kit may further comprise a divalent or trivalent metal chelator. In another embodiment, one component to form the bail out solution comprises an inorganic salt. In one embodiment, the inorganic salt releases an anion when dissolved to form the bail out solution, wherein the anion can chelate metals which may be present in the particles or microspheres, thus rapidly dissolving the particles or microspheres. In one embodiment, the inorganic salt releases citrate. In one embodiment, the enzyme loaded biologically derived particle is an alginate lyase loaded alginate particle comprising calcium wherein an anion, such as citrate, can chelate the calcium, leading to rapid dissolution of the particle. In another embodiment, one component to form the bail out solution comprises an inorganic salt which releases phosphate and/or sodium ions when dissolved to form the bail out solution. In one embodiment, the inorganic salt comprises the components to make phosphate buffered saline, when dissolved to form the bail out solution. In one embodiment, the kit comprises a solvent in which the components of the bail out solution are reconstituted, dissolved, suspended, or dispersed.

In one embodiment, the kit comprises an instruction booklet. In one embodiment, the instruction booklet provides information on how to reconstitute, dissolve, suspend, or disperse the particles or microspheres of the disclosure in the solvent to form a solution. In one embodiment, the instruction booklet provides information on how to administer a solution of the particles or microspheres to a subject in need thereof. In one embodiment, the kit comprises a syringe for administration of the solution or particles or microspheres. In one embodiment, the instruction booklet provides information on the dosage of particles or microspheres that should be administered to the subject in need thereof. In one embodiment, the booklet provides information on when the bail out solution should be administered to the subject and/or how to administer the bail out solution to the subject. In one embodiment, the booklet provides information on how to reconstitute, dissolve, suspend, or disperse the one or more components of the bail out solution in the solvent to form a solution. In one embodiment, the kit comprises a syringe for administration of the bail out solution.

### Other Embodiments and Equivalents

The section headings used herein are for organizational purposes only and are not to be construed as limiting the subject matter described.

It is to be understood that the methods described herein are not limited to the particular methodology, protocols, subjects, and sequencing techniques described herein and as such may vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment may be combined with any other embodiment. It should be understood that numerous specific details, relationships, and methods are set forth to provide a full understanding of the features described herein. A skilled artisan, however, will readily recognize that the features described herein may be practiced without one or more of the specific details or with other methods. The features described herein are not limited by the illustrated ordering of acts or events, as some acts may occur in different orders and/or concurrently with other acts or events. Furthermore, not all illustrated acts or events are required to implement a methodology in accordance with the features described herein. Further, to the extent that the methods of the present disclosure do not rely on the particular order of steps set forth herein, the particular order of the steps should not be construed as limitation on the claims. Any claims directed to the methods of the present disclosure should not be limited to the performance of their steps in the order written, and one skilled in the art may readily appreciate that the steps may be varied and still remain within the spirit and scope of the present disclosure.

While some embodiments have been shown and described herein, it will be obvious to those skilled in the art that such embodiments are provided by way of example only. It is not intended that embodiments of the present disclosure be limited by the specific examples provided within the specification. While certain embodiments of the present disclosure have been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the embodiments of the present disclosure are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define, at least in part, the scope of the invention and that methods and structures within the scope of these claims and their equivalents be covered thereby.

It will be appreciated by those skilled in the art that changes could be made to the exemplary embodiments shown and described above without departing from the broad inventive concepts thereof. It is understood, therefore, that this disclosure is not limited to the exemplary embodiments shown and described, but it is intended to cover modifications within the spirit and scope of the present disclosure as defined by the claims. For example, specific features of the exemplary embodiments may or may not be part of the claimed invention and various features of the disclosed embodiments may be combined. The words “right”, “left”, “lower” and “upper” designate directions in the drawings to which reference is made. The words “inwardly” and “outwardly” refer to directions toward and away from, respectively, the geometric center of the fluid delivery device. Unless specifically set forth herein, the terms “a”, “an” and “the” are not limited to one element but instead should be read as meaning “at least one”.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It is to be understood that at least some of the figures and descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

The following clauses describe certain embodiments of the disclosure.

Clause 1. An alginate microsphere capable of self-degradation upon rehydration, comprising:
an alginate lyase enzyme pre-treated by varying temperature, by varying pH, and/or with a metal-ion enzyme inhibitor;
alginate molecules having one or both of (i) a predetermined molecular weight, and (ii) a predetermined ratio of β-D-Mannuronic acid (M) blocks to α-L-Guluronic acid (G) blocks; and
a divalent metal-ion crosslinking the alginate molecules,
wherein the alginate microsphere is substantially free of water and/or sterilized.

Clause 2. The alginate microsphere of clause 1, wherein the degradation of alginate microsphere is controlled by one or more of the pre-treatment of the alginate lyase enzyme, amount of the alginate enzyme in the microsphere, the predetermined molecular weight of the alginate molecule, and the predetermined ratio of M:G blocks of the alginate molecules, and a composition of gelling bath, including an amount and or charge of one or more ions in the gelling bath.

Clause 3. The alginate microsphere of clause 1 or 2, wherein the pH of alginate lyase enzyme in the precursor solution containing alginate lyase and alginate, is in the range of pH 3.0 to 6.4, to prevent the degradation of alginate, before crosslinking with divalent metal cation.

Clause 4. The alginate microsphere of any one of clauses 1-3, wherein the metal-ion enzyme inhibitor is a reversible inhibitor selected from the group consisting of Cu²⁺, Zn²⁺, and Fe³⁺ (e.g., to control the degradation of alginate in the precursor solution, before crosslinking with divalent metal cation).

Clause 5. The alginate microsphere of any one of clauses 1-4, wherein the temperature of the precursor solution is in the range of 1-4° C. to control the degradation of alginate, before crosslinking with divalent metal cation.

Clause 6. The alginate microsphere of any one of clauses 1-5, wherein the pre-treatment of the alginate enzyme in the precursor solution allows mixing of a predetermined amount of enzyme (measured in units, U) with the alginate molecules.

Clause 7. The alginate microsphere of any one of clauses 1-6, wherein an activity of the alginate lyase enzyme is modulated by adjusting one or more of a pH of the gelling bath, a temperature of the gelling bath, and an amount of the metal-ion enzyme inhibitor in the alginate microsphere.

Clause 8. The alginate microsphere of any one of clauses 1-7, wherein the degradation of the alginate microsphere is controlled by the predetermined molecular weight of alginate molecules.

Clause 9. The alginate microsphere of any one of clauses 1-8, wherein the predetermined molecular weight of the alginate molecules is in a range of greater than about 100 kDa to less than about 800 kDa.

Clause 10. The alginate microsphere of any one of clauses 1-9, wherein the predetermined ratio of M:G block controls a degradation of alginate microsphere.

Clause 11. The alginate microsphere of any one of clauses 1-10, wherein the predetermined ratio of M:G blocks is about 50:50, about 55:45, about 60:40, about 65:35, about 70:30, about 75:25, about 80:20, about 85:15, about 90:10, or about 95:5.

Clause 12. The alginate microsphere of clause 11, wherein the alginate microsphere degrades over a period of less than about 5 days.

Clause 13. The alginate microsphere of 11 or 12, wherein the alginate microsphere degrades over a period of greater than about 2 days.

Clause 14. The alginate microsphere of any one of clauses 1-10, wherein the predetermined ratio of M:G blocks is about 50:50, about 45:55, about 40:60, about 35:65, about 30:70, about 25:75, about 20:80, about 15:85, about 10:90, or about 5:95.

Clause 15. The alginate microsphere of clause 14, wherein the alginate microsphere degrades over a period of between about 5 days and about 30 days.

Clause 16. The alginate microsphere of any one of clauses 1-15, wherein the pre-treated alginate lyase enzyme is mixed in the precursor solution having enzyme activity ranging from 0.025 U/mg to 1 U/mg of alginate.

Clause 17. The alginate microsphere of any one of clauses 1-16, wherein the activity of the alginate lyase enzyme is between about 0.05 mU (milliunits) and about 2.5 mU per microsphere.

Clause 18. The alginate microsphere of clause 17, wherein the alginate microsphere degrades over a period of less than about 5 days.

Clause 19. The alginate microsphere of any one of clauses 1-16, wherein the activity of the alginate lyase enzyme is between about 0.05 nU (nanounits) and about 0.05 mU per microsphere.

Clause 20. The alginate microsphere of clause 19, wherein the alginate microsphere degrades over a period of between about 5 days and about 30 days.

Clause 21. The alginate microsphere of any one of clauses 1-20, further comprising a bioactive agent.

Clause 22. The alginate microsphere of clause 21, wherein the bioactive agent comprises an anti-inflammatory agent and/or an anesthetic drug to alleviate pain associated with embolization in a subject.

Clause 23. The alginate microsphere of clause 21, wherein the bioactive agent comprises an anti-cancer agent, or an anti-angiogenic agent.

Clause 24. The alginate microsphere of clause 22, wherein the anti-inflammatory agent comprises hyaluronic acid having a molecular weight of between about 1 million (M) and about 5 M Daltons.

Clause 25. The alginate microsphere of clause 24, where in the ratio of hyaluronic acid to the alginate molecules is about 1:20 by weight.

Clause 26. The alginate microsphere of any one of clauses 1-25, further comprising a cryoprotectant selected from the group consisting of hydroxypropyl-β cyclodextrin, trehalose, polyvinyl pyrrolidone of 40 kDa (PVP 40 kDa) and dextran (70 kDa molecular weight).

Clause 27. The alginate microsphere of any one of clauses 1-26, wherein the alginate microsphere is lyophilized.

Clause 28. The alginate microsphere of clause 27, wherein a residual water content of the lyophilized alginate microsphere is in the range of about 1% to about 3% by mass.

Clause 29. The alginate microsphere of any one of clauses 1-28, wherein a sphericity of the alginate microsphere is at least about 0.7, at least about 0.75, at least about 0.8, at least about 0.85, at least about 0.9, at least about 0.95, or at least about 0.99.

Clause 30. The alginate microsphere of any one of clauses 1-29, wherein the alginate microsphere or the lyophilized alginate microsphere is sterilized.

Clause 31. The alginate microsphere of clause 30, wherein the sterilization comprises high energy radiation sterilization, gamma-ray sterilization, or e-beam sterilization.

Clause 32. The alginate microsphere of clause 31, wherein the sterilization comprises between about 15 and about 25 kGy of gamma radiation from Cobalt 60 Isotope, or about 25 kGy of electron beam radiation in accordance with ISO 11137-1:2006.

Clause 33. The alginate microspheres of any one of clauses 1-31, wherein a shelf-life of the alginate microsphere is at least about 3 months, at least about 6 months, at least about 12 months, at least about 18 months, at least about 24 months, at least about 36 months, at least about 48 months, or at least about 60 months when stored at a given temperature.

Clause 34. The alginate microsphere of clause 33, wherein the given temperature is between about 2° C. and about 8° C.

Clause 35. The alginate microsphere of clause 33, wherein the given temperature is about room temperature (RT).

Clause 36. The alginate microsphere of any one of clauses 27-35, wherein the alginate microsphere is reconstituted in saline or saline-radiopaque contrast at physiological pH.

Clause 37. A method of preparing an alginate microsphere capable of self-degradation upon rehydration, the method comprising:
forming droplets from a precursor solution, the precursor solution comprising: an alginate lyase enzyme pre-treated by varying temperature, by varying pH, and/or with a metal-ion enzyme inhibitor; and alginate molecules having one or both of (a) a predetermined molecular weight, and (b) a predetermined ratio of β-D-Mannuronic acid (M) blocks to α-L-Guluronic acid (G) blocks;
contacting the droplets with a gelling bath comprising a cryoprotectant, and a divalent metal-ion, thereby crosslinking the alginate molecules to form an alginate microsphere; and
dehydrating, and optionally sterilizing, the alginate microsphere thereby substantially removing water from the microsphere.

Clause 38. The method of clause 37, wherein the precursor solution comprises one or more cryoprotectants.

Clause 39. The method of clause 37 or 38, wherein the gelling bath comprises one or more cryoprotectants.

Clause 40. The method of clause 38 or 39, wherein the cryoprotectant is selected from the group consisting of hydroxypropyl-β cyclodextrin, trehalose, polyvinyl pyrrolidone of 40 kDa (PVP 40 kDa) and dextran (70 kDa molecular weight).

Clause 41. The method of clause 40, wherein the concentration of the trehalose in the precursor solution is about 0.1% w/v to about 20% w/v.

Clause 42. The method of clause 40 or 41, wherein the concentration of the hydroxypropyl-β cyclodextrin is about 0.1% w/v to about 2% w/v.

Clause 43. The method of any one of clauses 40-42, wherein the concentration of the PVP 40 kDa in the precursor solution is about 0.1% w/v to about 1% w/v.

Clause 44. The method of any one of clauses 40-43, wherein the concentration of the dextran (molecular weight 70 kDa) in the precursor solution is about 0.1% w/v to about 1% w/v.

Clause 45. The method of any one of clauses 39-44, wherein the precursor solution and the gelling bath comprise the same cryoprotectant.

Clause 46. The method of clause 45, wherein the precursor solution and the gelling bath comprise the same cryoprotectant at equal or about equal concentrations.

Clause 47. The method of any one of clauses 37-46, wherein the dehydrating comprises lyophilizing the alginate microsphere.

Clause 48. The method of clause 47, wherein a residual water content of the lyophilized alginate microsphere is in the range of about 1% to about 3% by mass.

Clause 49. The method of any one of clauses 37-48, wherein a sphericity of the alginate microspheres is at least about 0.7, at least about 0.75, at least about 0.8, at least about 0.85, at least about 0.9, at least about 0.95, or at least about 0.99.

Clause 50. The method of any one of clauses 37-49, further comprising sterilizing the alginate microsphere or lyophilized alginate microsphere.

Clause 51. The method of clause 50, wherein the sterilizing comprises high energy radiation sterilization, gamma-ray sterilization, or e-beam sterilization.

Clause 52. The method of clause 51, wherein the sterilizing comprises between about 15 and about 25 kGy of gamma radiation from Cobalt 60 Isotope, or about 25 kGy of electron beam radiation in accordance with ISO 11137-1:2006.

Clause 53. The method of any one of clauses 37-51, further comprising storing the alginate microsphere for at least about 3 months, at least about 6 months, at least about 12 months, at least about 18 months, at least about 24 months, at least about 36 months, at least about 48 months, or at least about 60 months when stored at a given temperature.

Clause 54. The method of any one of clauses 37-53, wherein a shelf-life of the alginate microsphere is at least about 3 months, at least about 6 months, at least about 12 months, at least about 18 months, at least about 24 months, at least about 36 months, at least about 48 months, or at least about 60 months when stored at a given temperature.

Clause 55. The method of clause 53 or 54, wherein the given temperature is between about 2° C. and about 8° C.

Clause 56. The method of clause 53 or 54, wherein the given temperature is about room temperature (RT).

Clause 57. The method of any one of clauses 37-56, further comprising administering the alginate microsphere, the lyophilized alginate microsphere, or the sterilized microsphere to a subject.

Clause 58. The method of clause 57, wherein the step of administering the alginate microsphere, the lyophilized alginate microsphere, or the sterilized microsphere to the subject is preceded by reconstituting the lyophilized alginate microsphere, or the sterilized microsphere in saline or saline-radiopaque contrast at physiological pH.

Clause 59. The method of any one of clauses 37-58, wherein forming the droplets is performed using a method selected from the group consisting of drop casting, spray congealing/spray cooling, spray drying, microfluidic droplet production, and jet-cutting.

Clause 60. The method of any one of clauses 37-59, wherein the degradation of alginate microsphere is controlled by one or more of the pre-treatment of the alginate lyase enzyme, amount of the alginate enzyme in the microsphere, the predetermined molecular weight of the alginate molecule, and the predetermined ratio of M:G blocks of the alginate molecules, and a composition of gelling bath, including an amount and or charge of one or more ions in the gelling bath.

Clause 61. The method of any one of clauses 37-60, wherein the pH of alginate lyase enzyme in the precursor solution containing alginate lyase and alginate, is in the range of pH 3.0 to 6.4, to prevent the degradation of alginate, before crosslinking with divalent metal cation.

Clause 62. The method of any one of clauses 37-60, wherein the metal-ion enzyme inhibitor is a reversible inhibitor selected from the group consisting of Cu²⁺, Zn²⁺, and Fe³⁺ (e.g., to control the degradation of alginate in the precursor solution, before crosslinking with divalent metal cation).

Clause 63. The method of any one of clauses 37-62, wherein the temperature of the precursor solution is in the range of 1-4° C. to control the degradation of alginate, before crosslinking with divalent metal cation.

Clause 64. The method of any one of clauses 37-62, wherein the pre-treatment of the alginate enzyme in the precursor solution allows mixing of a predetermined amount of enzyme (measured in units, U) with the alginate molecules.

Clause 65. The method of any one of clauses 37-64, wherein an activity of the alginate lyase enzyme is modulated by adjusting one or more of a pH of the gelling bath, a temperature of the gelling bath, and an amount of the metal-ion enzyme inhibitor in the alginate microsphere.

Clause 66. The method of any one of clauses 37-64, wherein the degradation of the alginate microsphere is controlled by the predetermined molecular weight of alginate molecules.

Clause 67. The method of any one of clauses 37-66, wherein the predetermined molecular weight of the alginate molecules is in a range of greater than about 100 kDa to less than about 800 kDa.

Clause 68. The method of any one of clauses 37-67, wherein the predetermined ratio of M:G block controls a degradation of alginate microsphere.

Clause 69. The method of any one of clauses 37-68, wherein the predetermined ratio of M:G blocks is about 50:50, about 55:45, about 60:40, about 65:35, about 70:30, about 75:25, about 80:20, about 85:15, about 90:10, or about 95:5.

Clause 70. The method of clause 69, wherein the alginate microsphere degrades over a period of less than about 5 days.

Clause 71. The method of clause 69 or 70, wherein the alginate microsphere degrades over a period of greater than about 2 days.

Clause 72. The method of any one of clauses 37-68, wherein the predetermined ratio of M:G blocks is about 50:50, about 45:55, about 40:60, about 35:65, about 30:70, about 25:75, about 20:80, about 15:85, about 10:90, or about 5:95.

Clause 73. The method of clause 72, wherein the alginate microsphere degrades over a period of between about 5 days and about 30 days.

Clause 74. The method of any one of clauses 37-73, wherein the pre-treated alginate lyase enzyme is mixed in the precursor solution having enzyme activity ranging from 0.0025 U/mg to 1 U/mg of alginate.

Clause 75. The method of any one of clauses 37-74, wherein the pre-treated alginate lyase enzyme is mixed in the precursor solution having enzyme activity ranging from 0.125 U/mg to 0.250 U/mg of alginate.

Clause 76. The method of clause 75, wherein the alginate microsphere degrades over a period of less than about 5 days.

Clause 77. The method of any one of clauses 37-74, wherein the pre-treated alginate lyase enzyme is mixed in the precursor solution having enzyme activity ranging from 0.025 U/mg to 0.125 U/mg of alginate.

Clause 78. The method of clause 77, wherein the alginate microsphere degrades over a period of between about 5 days and about 30 days.

Clause 79. The method of any one of clauses 37-74, wherein the pre-treated alginate lyase enzyme is mixed in the precursor solution having enzyme activity ranging from 0.0025 U/mg to 0.005 U/mg of alginate.

Clause 80. The method of clause 79, wherein the alginate microsphere degrades over a period of greater than about 30 days.

Clause 81. The method of any one of clauses 37-80, wherein the precursor solution and/or the gelling bath further comprises a bioactive agent.

Clause 82. The method of clause 81, wherein the bioactive agent comprises an anti-inflammatory agent and/or an anesthetic agent to alleviate pain associated with embolization in a subject.

Clause 83. The method of clause 81, wherein the bioactive agent comprises an anti-cancer agent, or an anti-angiogenic agent.

Clause 84. The method of clause 83, wherein the anti-inflammatory agent comprises hyaluronic acid having a molecular weight of between about 1 million (M) and about 5 M Daltons.

Clause 85. The method of clause 84, wherein the ratio of hyaluronic acid to the alginate molecules is about 1:20 by weight.

Clause 86. The method of any one of clauses 37-85, wherein a pH of the gelling bath is less than about 6.5.

Clause 87. The method of any one of clauses 37-85, wherein a pH of the gelling bath is equal to or about equal to a pH of the precursor solution.

Clause 88. The method of any one of clauses 37-87, wherein a temperature of the precursor solution is equal to or about equal to between 1° C. and about 4° C.

Clause 89. A photopolymerized, alginate microsphere capable of self-degradation upon rehydration, comprising:
an alginate lyase enzyme pre-treated by varying temperature, by varying pH, and/or with a metal-ion enzyme inhibitor;
alginate molecules functionalized with an ethylenically unsaturated functional group, the molecules having one or both of (i) a predetermined molecular weight, and (ii) a predetermined ratio of β-D-Mannuronic acid (M) blocks to α-L-Guluronic acid (G) blocks; and
a photoinitiator,
wherein the alginate molecules are crosslinked by irradiating the photoinitiator, and
wherein the alginate microsphere is substantially free of water and/or sterilized.

Clause 90. The alginate microsphere of clause 89, wherein the ethylenically unsaturated functional group is selected from the group consisting of acrylate, methacrylate, vinylic, and allylic.

Clause 91. The alginate microsphere of clause 89 or 90, wherein the degradation of alginate microsphere is controlled by one or more of the pre-treatment of the alginate lyase enzyme, the predetermined molecular weight of the alginate molecule, and the predetermined ratio of M:G blocks of the alginate molecules, and a composition of gelling bath, including an amount and or charge of one or more ions in the gelling bath.

Clause 92. The alginate microsphere of any one of clauses 89-91, wherein the pH of alginate lyase enzyme in the precursor solution containing alginate lyase and alginate, is in the range of pH 3.0 to 6.4, to prevent the degradation of alginate, before crosslinking with divalent metal cation.

Clause 93. The alginate microsphere of any one of clauses 89-91, wherein the metal-ion enzyme inhibitor is a reversible inhibitor selected from the group consisting of Cu²⁺, Zn²⁺, and Fe³⁺ (e.g., to control the degradation of alginate in the precursor solution, before crosslinking with divalent metal cation).

Clause 94. The alginate microsphere of any one of clauses 89-93, wherein the temperature of the precursor solution is in the range of 1-4° C. to control the degradation of alginate, before crosslinking with divalent metal cation.

Clause 95. The alginate microsphere of any one of clauses 89-93, wherein the pre-treatment of the alginate enzyme in the precursor solution allows mixing of a predetermined amount of enzyme (measured in units, U) with the alginate molecule.

Clause 96. The alginate microsphere of any one of clauses 89-95, wherein an activity of the alginate lyase enzyme is modulated by adjusting one or more of a pH of the gelling bath, a temperature of the gelling bath, and an amount of the metal-ion enzyme inhibitor in the alginate microsphere.

Clause 97. The alginate microsphere of any one of clauses 89-96, wherein the degradation of the alginate microsphere is controlled by the predetermined molecular weight of alginate molecules.

Clause 98. The alginate microsphere of any one of clauses 89-96, wherein the predetermined molecular weight of the alginate molecules is in a range of greater than about 100 kDa to less than about 800 kDa.

Clause 99. The alginate microsphere of any one of clauses 89-98, wherein the predetermined ratio of M:G block controls a degradation of alginate microsphere.

Clause 100. The alginate microsphere of any one of clauses 89-99, wherein the predetermined ratio of M:G blocks is about 50:50, about 55:45, about 60:40, about 65:35, about 70:30, about 75:25, about 80:20, about 85:15, about 90:10, or about 95:5.

Clause 101. The alginate microsphere of clause 100, wherein the alginate microsphere degrades over a period of less than about 5 days.

Clause 102. The alginate microsphere of clause 100 or 101, wherein the alginate microsphere degrades over a period of greater than about 2 days.

Clause 103. The alginate microsphere of any one of clauses 89-99, wherein the predetermined ratio of M:G blocks is about 50:50, about 45:55, about 40:60, about 35:65, about 30:70, about 25:75, about 20:80, about 15:85, about 10:90, or about 5:95.

Clause 104. The alginate microsphere of clause 103, wherein the alginate microsphere degrades over a period of between about 5 days and about 30 days.

Clause 105. The alginate microsphere of any one of clauses 89-104, wherein the pre-treated alginate lyase enzyme is mixed in the precursor solution having enzyme activity ranging from 0.025 U/mg to 1 U/mg of alginate.

Clause 106. The alginate microsphere of any one of clauses 89-105, wherein the activity of the alginate lyase enzyme is between about 0.05 mU (milliunits) and about 2.5 mU per microsphere.

Clause 107. The alginate microsphere of clause 106, wherein the alginate microsphere degrades over a period of less than about 5 days.

Clause 108. The alginate microsphere of any one of clauses 89-105, wherein the activity of the alginate lyase enzyme is between about 0.05 nU (nanounits) and about 0.05 mU per microsphere.

Clause 109. The alginate microsphere of clause 108, wherein the alginate microsphere degrades over a period of between about 5 days and about 30 days.

Clause 110. The alginate microsphere of any one of clauses 89-109, further comprising a bioactive agent.

Clause 111. The alginate microsphere of clause 110, wherein the bioactive agent comprises an anti-inflammatory agent to alleviate pain associated with embolization in a subject.

Clause 112. The alginate microsphere of clause 111, wherein the anti-inflammatory agent comprises hyaluronic acid having a molecular weight of between about 1 million (M) and about 5 M Daltons.

Clause 113. The alginate microsphere of clause 112, wherein the ratio of hyaluronic acid to the alginate molecules is about 1:20 by weight.

Clause 114. The alginate microsphere of any one of clauses 89-113, further comprising a cryoprotectant selected from the group consisting of hydroxypropyl-β cyclodextrin, trehalose, polyvinyl pyrrolidone of 40 kDa (PVP 40 kDa) and dextran (70 kDa molecular weight).

Clause 115. The alginate microsphere of any one of clauses 89-114, wherein the alginate microsphere is lyophilized.

Clause 116. The alginate microsphere of clause 103, wherein a residual water content of the lyophilized alginate microsphere is in the range of about 1% to about 3% by mass.

Clause 117. The alginate microsphere of any one of clauses 89-116, wherein a sphericity of the alginate microsphere is at least about 0.7, at least about 0.75, at least about 0.8, at least about 0.85, at least about 0.9, at least about 0.95, or at least about 0.99.

Clause 118. The alginate microsphere of any one of clauses 89-117, wherein the alginate microsphere or the lyophilized alginate microsphere is sterilized.

Clause 119. The alginate microsphere of clause 118, wherein the sterilization comprises high energy radiation sterilization, gamma-ray sterilization, or e-beam sterilization.

Clause 120. The alginate microsphere of clause 119, wherein the sterilization comprises between about 15 and about 25 kGy of gamma radiation from Cobalt 60 Isotope, or about 25 kGy of electron beam radiation in accordance with ISO 11137-1:2006.

Clause 121. The alginate microsphere of any one of clauses 89-120, wherein a shelf-life of the alginate microsphere is at least about 3 months, at least about 6 months, at least about 12 months, at least about 18 months, at least about 24 months, at least about 36 months, at least about 48 months, or at least about 60 months when stored at a given temperature.

Clause 122. The alginate microsphere of clause 121, wherein the given temperature is between about 2° C. and about 8° C.

Clause 123. The alginate microsphere of clause 121, wherein the given temperature is about room temperature (RT).

Clause 124. The alginate microsphere of any one of clauses 115-123, wherein the alginate microsphere is reconstituted in saline or saline-radiopaque contrast at physiological pH.

Clause 125. A method of preparing a photopolymerized, alginate microsphere capable of self-degradation upon rehydration, the method comprising:
forming droplets from a precursor solution, the precursor solution comprising: an alginate lyase enzyme pre-treated by varying temperature, by varying pH, and/or with a metal-ion enzyme inhibitor; alginate molecules functionalized with an ethylenically unsaturated functional group, the molecules having one or both of (a) a predetermined molecular weight, and (b) a predetermined ratio of β-D-Mannuronic acid (M) blocks to α-L-Guluronic acid (G) blocks; and a photoinitiator;
irradiating the droplets including the photoinitiator, thereby crosslinking the alginate molecules to form a photopolymerized, alginate microsphere; and
dehydrating, and optionally sterilizing, the alginate microsphere thereby substantially removing water from the microsphere.

Clause 126. The method of clause 125, wherein the ethylenically unsaturated functional group is selected from the group consisting of acrylate, methacrylate, vinylic, and allylic.

Clause 127. The method of clause 125 or 126, wherein the precursor solution comprises one or more cryoprotectants.

Clause 128. The method of any one of clauses 125-127, wherein the gelling bath comprises one or more cryoprotectants.

Clause 129. The method of clause 127 or 128, wherein the cryoprotectant is selected from the group consisting of hydroxypropyl-β cyclodextrin, trehalose, polyvinyl pyrrolidone of 40 kDa (PVP 40 kDa) and dextran (70 kDa molecular weight).

Clause 130. The method of clause 129, wherein the concentration of the trehalose in the precursor solution is about 0.1% w/v to about 20% w/v.

Clause 131. The method of clause 129 or 130, wherein the concentration of the Hydroxypropyl-β cyclodextrin is about 0.1% w/v to about 2% w/v.

Clause 132. The method of any one of clauses 129-131, wherein the concentration of the PVP 40 kDa in the precursor solution is about 0.1% w/v to about 1% w/v.

Clause 133. The method of any one of clauses 129-132, wherein the concentration of the dextran (molecular weight 70 kDa) in the precursor solution is about 0.1% w/v to about 1% w/v.

Clause 134. The method of any one of clauses 128-133, wherein the precursor solution and the gelling bath comprise the same cryoprotectant.

Clause 135. The method of clause 134, wherein the precursor solution and the gelling bath comprise the same cryoprotectant at equal or about equal concentrations.

Clause 136. The method of any one of clauses 125-135, wherein the dehydrating comprises lyophilizing the alginate microsphere.

Clause 137. The method of clause 136, wherein a residual water content of the lyophilized alginate microsphere is in the range of about 1% to about 3% by mass.

Clause 138. The method of any one of clauses 125-137, wherein a sphericity of the alginate microsphere is at least about 0.7, at least about 0.75, at least about 0.8, at least about 0.85, at least about 0.9, at least about 0.95, or at least about 0.99.

Clause 139. The method of any one of clauses 125-138, further comprising sterilizing the alginate microsphere or lyophilized alginate microsphere.

Clause 140. The method of clause 139, wherein the sterilizing comprises high energy radiation sterilization, gamma-ray sterilization, or e-beam sterilization.

Clause 141. The alginate microsphere of clause 140, wherein the sterilization comprises between about 15 and about 25 kGy of gamma radiation from Cobalt 60 Isotope, or about 25 kGy of electron beam radiation in accordance with ISO 11137-1:2006.

Clause 142. The method of any one of clause 125-141, further comprising storing the alginate microsphere for at least about 3 months, at least about 6 months, at least about 12 months, at least about 18 months, at least about 24 months, at least about 36 months, at least about 48 months, or at least about 60 months when stored at a given temperature.

Clause 143. The method of any one of clauses 125-142, wherein a shelf-life of the alginate microsphere is at least about 3 months, at least about 6 months, at least about 12 months, at least about 18 months, at least about 24 months, at least about 36 months, at least about 48 months, or at least about 60 months when stored at a given temperature.

Clause 144. The method of clause 142 or 143, wherein the given temperature is between about 2° C. and about 8° C.

Clause 145. The method of clause 142 or 143, wherein the given temperature is about room temperature (RT).

Clause 146. The method of any one of clauses 125-145, further comprising administering the alginate microsphere, the lyophilized alginate microsphere, or the sterilized microsphere to a subject.

Clause 147. The method of clause 146, wherein the step of administering the alginate microsphere, the lyophilized alginate microsphere, or the sterilized microsphere to a subject is preceded by reconstituting the alginate microsphere, the lyophilized alginate microsphere, or the sterilized microsphere using saline or saline-radiopaque contrast at physiological pH.

Clause 148. The method of any one of clauses 125-147, wherein flowing the precursor solution through the orifice to form droplets is performed using a method selected from the group consisting of drop casting, spray congealing/spray cooling, spray drying, and microfluidic droplet production.

Clause 149. The method of any one of clauses 125-148, wherein the degradation of alginate microsphere is controlled by one or more of the pre-treatment of the alginate lyase enzyme, the predetermined molecular weight of the alginate molecule, and the predetermined ratio of M:G blocks of the alginate molecules, and a composition of gelling bath, including an amount and or charge of one or more ions in the gelling bath.

Clause 150. The method of any one of clauses 125-149, wherein the pH of alginate lyase enzyme in the precursor solution containing alginate lyase and alginate, is in the range of pH 3.0 to 6.4, to prevent the degradation of alginate, before crosslinking with divalent metal cation.

Clause 151. The method of any one of clauses 125-150, wherein the metal-ion enzyme inhibitor is a reversible inhibitor selected from the group consisting of Cu²⁺, Zn²⁺, and Fe³⁺ (e.g., to control the degradation of alginate in the precursor solution, before crosslinking with divalent metal cation).

Clause 152. The method of any one of clauses 125-151, wherein the temperature of the precursor solution is in the range of 1-4° C. to control the degradation of alginate, before crosslinking with divalent metal cation.

Clause 153. The method of any one of clauses 125-152, wherein the pre-treatment of the alginate enzyme in the precursor solution allow the mixing of the desired activity of enzyme (referred as units, U) with the alginate, which controls a degradation of the alginate microsphere for a length of time.

Clause 154. The method of any one of clauses 125-153, wherein the degradation of the alginate microsphere is controlled by the predetermined molecular weight of alginate molecules.

Clause 155. The method of any one of clauses 125-154, wherein the predetermined molecular weight of the alginate molecules is in a range of greater than about 100 kDa to less than about 800 kDa.

Clause 156. The method of any one of clauses 125-155, wherein the predetermined ratio of M:G block controls a degradation of alginate microsphere.

Clause 157. The method of any one of clauses 125-156, wherein the predetermined ratio of M:G blocks is about 50:50, about 55:45, about 60:40, about 65:35, about 70:30, about 75:25, about 80:20, about 85:15, about 90:10, or about 95:5.

Clause 158. The method of clause 157, wherein the alginate microsphere degrades over a period of less than about 5 days.

Clause 159. The method of clause 157 or 158, wherein the alginate microsphere degrades over a period of greater than about 2 days.

Clause 160. The method of any one of clauses 125-156, wherein the predetermined ratio of M:G blocks is about 50:50, about 45:55, about 40:60, about 35:65, about 30:70, about 25:75, about 20:80, about 15:85, about 10:90, or about 5:95.

Clause 161. The method of clause 160, wherein the alginate microsphere degrades over a period of between about 5 days and about 30 days.

Clause 162. The method of any one of clauses 125-161, wherein the pre-treated alginate lyase enzyme is mixed in the precursor solution having enzyme activity ranging from 0.0025 U/mg to 1 U/mg of alginate.

Clause 163. The method of any one of clauses 125-162, wherein the pre-treated alginate lyase enzyme is mixed in the precursor solution having enzyme activity ranging from 0.125 U/mg to 0.250 U/mg of alginate.

Clause 164. The method of clause 163, wherein the alginate microsphere degrades over a period of less than about 5 days.

Clause 165. The method of any one of clauses 125-162, wherein the pre-treated alginate lyase enzyme is mixed in the precursor solution having enzyme activity ranging from 0.025 U/mg to 0.125 U/mg of alginate.

Clause 166. The method of clause 165, wherein the alginate microsphere degrades over a period of between about 5 days and about 30 days.

Clause 167. The method of any one of clauses 124-147, the pre-treated alginate lyase enzyme is mixed in the precursor solution having enzyme activity ranging from 0.0025 U/mg to 0.005 U/mg of alginate.

Clause 168. The method of clause 167, wherein the alginate microsphere degrades over a period of greater than about 30 days.

Clause 169. The method of any one of clauses 125-168, wherein the precursor solution and/or the gelling bath further comprises a bioactive agent.

Clause 170. The method of clause 169, wherein the bioactive agent comprises an anti-inflammatory agent to alleviate pain associated with embolization in a subject.

Clause 171. The method of clause 170, wherein the anti-inflammatory agent comprises hyaluronic acid having a molecular weight of between about 1 million (M) and about 5 M Daltons.

Clause 172. The method of clause 171, wherein the ratio of hyaluronic acid to the alginate molecules is about 1:20 by weight.

Clause 173. The method of any one of clauses 125-172, wherein a pH of the gelling bath is less than about 6.5.

Clause 174. The method of any one of clauses 125-172, wherein a pH of the gelling bath is equal to or about equal to a pH of the precursor solution.

Clause 175. The method of any one of clauses 125-174, wherein a temperature of the precursor solution is equal to or about equal to between 1° C. and about 4° C.

Clause 176. A method of inducing a self-degrading embolism in a subject in need thereof, comprising administering a plurality of the alginate microspheres of any one of clauses 1-36 and 89-124 into a blood vessel of the subject.

Clause 177. The method of clause 176, wherein the blood vessel is a geniculate artery. Clause 178. A syringe, comprising:
a first chamber comprising alginate microspheres of any one of clauses 1-36 or 89-124;
a second chamber disposed axially to the first chamber, said second chamber comprising a reconstitution medium; and
a plunger configured to, upon depression, expose the alginate microspheres to the reconstitution medium, thereby reconstituting the alginate microspheres.

Clause 179. The syringe of clause 178, further comprising a breakable membrane separating the first chamber and the second chamber, wherein upon depression of the plunger, the breakable membrane breaks to expose the alginate microspheres to the reconstitution medium, thereby reconstituting the alginate microspheres.

Clause 180. A microsphere capable of self-degradation, upon rehydration, for administration to a mammalian subject in need thereof, the microsphere comprising:
a biocompatible polysaccharide material incapable of being enzymatically hydrolyzed by the mammalian subject, wherein the biocompatible polysaccharide material has one or both of (i) a predetermined molecular weight, and (ii) a predetermined ratio of β-D-Mannuronic acid (M) blocks to α-L-Guluronic acid (G) blocks;
an enzyme capable of hydrolyzing the biocompatible polysaccharide material, wherein the enzyme does not naturally occur in the mammalian subject, and wherein the enzyme is pre-treated by varying temperature, by varying pH, and/or with a metal-ion enzyme inhibitor; and a divalent metal-ion crosslinking the biocompatible polysaccharide material, wherein the microsphere is substantially free of water and/or sterilized.

Clause 181. The microsphere of clause 180, wherein the biocompatible polysaccharide material comprises alginate.

Clause 182. The microsphere of clause 180 or 181, wherein the enzyme comprises alginate lyase.

Clause 183. The microsphere of any one of clauses 180-182, wherein the biocompatible polysaccharide material is resorbable.

Clause 184. The microsphere of any one of clauses 180-183, wherein the biocompatible polysaccharide material is stable to enzymatic hydrolysis within the mammalian subject.

Clause 185. The microsphere of any one of clauses 180-184, wherein a rate of resorption of the biocompatible polysaccharide material is more precisely controlled by inclusion of a quantity of an enzyme not found within the mammal that has a specific action that causes breakdown of the embolic material once in the body.

Clause 186. A method of preparing a microsphere capable of self-degradation, upon rehydration, for administration to a mammalian subject in need thereof, the method comprising:
forming droplets from a precursor solution, the precursor solution comprising: a biocompatible polysaccharide material incapable of being enzymatically hydrolyzed by the mammalian subject, wherein the biocompatible polysaccharide material has one or both of (i) a predetermined molecular weight, and (ii) a predetermined ratio of β-D-Mannuronic acid (M) blocks to α-L-Guluronic acid (G) blocks; and an enzyme capable of hydrolyzing the biocompatible polysaccharide material, wherein the enzyme does not naturally occur in the mammalian subject, and wherein the enzyme is pre-treated by varying temperature, by varying pH, and/or with a metal-ion enzyme inhibitor;
contacting the droplets with a gelling bath comprising a cryoprotectant, and a divalent metal-ion, thereby crosslinking the biocompatible polysaccharide material to form a microsphere,
dehydrating, and optionally sterilizing, the microsphere thereby substantially removing water from the microsphere.

Clause 187. The method of clause 186, wherein the biocompatible polysaccharide material comprises alginate.

Clause 188. The method of clause 186 or 187, wherein the enzyme comprises alginate lyase.

Clause 189. The method of any one of clauses 186-188, wherein the biocompatible polysaccharide material is resorbable.

Clause 190. The method of any one of clauses 186-189, wherein the biocompatible polysaccharide material is stable to enzymatic hydrolysis within the mammalian subject.

Clause 191. The method of any one of clauses 186-190, wherein a rate of resorption of the biocompatible polysaccharide material is more precisely controlled by inclusion of a quantity of an enzyme not found within the mammal that has a specific action that causes breakdown the embolic material once in the body.

Clause 192. A photopolymerized, microsphere capable of self-degradation, upon rehydration, for administration to a mammalian subject in need thereof, comprising:
a biocompatible polysaccharide material incapable of being enzymatically hydrolyzed by the mammalian subject, wherein the biocompatible polysaccharide material has one or both of (i) a predetermined molecular weight, and (ii) a predetermined ratio of β-D-Mannuronic acid (M) blocks to α-L-Guluronic acid (G) blocks;
an enzyme capable of hydrolyzing the biocompatible polysaccharide material, wherein the enzyme does not naturally occur in the mammalian subject, and wherein the enzyme is pre-treated by varying temperature, by varying pH, and/or with a metal-ion enzyme inhibitor; and
a photoinitiator,
wherein the biocompatible polysaccharide material is crosslinked by irradiating the photoinitiator, and
wherein the microsphere is substantially free of water and/or sterilized.

Clause 193. The microsphere of clause 192, wherein the biocompatible polysaccharide material comprises alginate.

Clause 194. The microsphere of clause 192 or 193, wherein the enzyme comprises alginate lyase.

Clause 195. The microsphere of any one of clauses 192-194, wherein the biocompatible polysaccharide material is resorbable.

Clause 196. The microsphere of any one of clauses 192-195, wherein the biocompatible polysaccharide material is stable to enzymatic hydrolysis within the mammalian subject.

Clause 197. The microsphere of any one of clauses 192-196, wherein a rate of resorption of the biocompatible polysaccharide material is more precisely controlled by inclusion of a quantity of an enzyme not found within the mammal that has a specific action that causes breakdown of the embolic material once in the body.

Clause 198. A method of preparing a photopolymerized, microsphere capable of self-degradation, upon rehydration, for administration to a mammalian subject in need thereof, the method comprising:
flowing a precursor solution through an orifice to form droplets, the precursor solution comprising: a biocompatible polysaccharide material incapable of being enzymatically hydrolyzed by the mammalian subject, wherein the biocompatible polysaccharide material has one or both of (i) a predetermined molecular weight, and (ii) a predetermined ratio of β-D-Mannuronic acid (M) blocks to α-L-Guluronic acid (G) blocks; an enzyme capable of hydrolyzing the biocompatible polysaccharide material, wherein the enzyme does not naturally occur in the mammalian subject, and wherein the enzyme is pre-treated by varying temperature, by varying pH, and/or with a metal-ion enzyme inhibitor; and a photoinitiator; and
irradiating the droplets including the photoinitiator, thereby crosslinking the biocompatible polysaccharide material to form a photopolymerized, microsphere,
dehydrating, and optionally sterilizing, the microsphere thereby substantially removing water from the microsphere.

Clause 199. The method of clause 198, wherein the biocompatible polysaccharide material comprises alginate.

Clause 200. The method of clause 198 or 199, wherein the enzyme comprises alginate lyase.

Clause 201. The method of any one of clauses 198-200, wherein the biocompatible polysaccharide material is resorbable.

Clause 202. The method of any one of clauses 198-201, wherein the biocompatible polysaccharide material is stable to enzymatic hydrolysis within the mammalian subject.

Clause 203. The method of any one of clauses 198-202, wherein the rate of resorption of the biocompatible polysaccharide material is more precisely controlled by inclusion of a quantity of an enzyme not found within the mammal that has a specific action that causes breakdown the embolic material once in the body.

Clause 300. A method of preparing an alginate microsphere capable of self-degradation upon rehydration, the method comprising:
forming droplets from a precursor solution using a microfluidics platform, the precursor solution comprising:
   (i) an alginate lyase enzyme pre-treated by alkaline pH solution of varying range and varying temperature (1-4° C.);
   (ii) alginate molecules having one or both of (a) a predetermined molecular weight, and (b) a predetermined ratio of β-D-Mannuronic acid (M) blocks to α-L-Guluronic acid (G) blocks;
   (iii) crosslinking agent such as Ca-EDTA or CaCO₃ and;
   (iv) excipients
contacting the droplets with a gelling solution comprising of surfactant, oil, acetic acid, thereby cross-linking the alginate molecules to form an alginate microsphere; and
dehydrating, and optionally sterilizing, the alginate microsphere thereby substantially removing water from the microsphere.

Clause 301. An alginate microsphere made by the method of clause 300.

Clause 302. The alginate microsphere of claims 301, wherein the degradation of alginate microsphere is controlled by one or more of the pre-treatment of the alginate lyase enzyme, amount of the alginate enzyme in the microsphere, the predetermined molecular weight of the alginate molecule, and the predetermined ratio of M:G blocks of the alginate molecules, and bivalent cation-crosslinking.

Clause 303. The alginate microsphere of clause 302, wherein the pH of precursor solution containing Alginate, alginate lyase and crosslinking agent (Ca-EDTA or CaCO₃) is the range of 8 to 13 at temperature 1-4° C. to prevent the degradation of alginate as well as the gelation of precursor solution.

Clause 304. The alginate microsphere of any one of clauses 301-303, wherein the pre-treatment of the alginate enzyme in the precursor solution allows mixing of a predetermined amount of enzyme (measured in units, U) with the alginate molecules.

Clause 305. The alginate microsphere any one of clauses 301-304, wherein after the droplet of the precursor solution have alkaline pH and low temperature can be generation through microfluidics chip and crosslinked in a solution containing acetic acidic in the range 0.05% v/v to 5% v/v, oil and surfactant for a duration of 1 min to 3 hours. Under acidic condition, the crosslinking of alginate with Ca²⁺ ion occur due to the ionization of Ca-EDTA or CaCO₃ and the encapsulated enzyme remains inactive under acidic pH condition, thus preventing the degradation of alginate beads.

Clause 306. The alginate microsphere any one of clauses 301-305, wherein the Ca²⁺-crosslinked alginate beads containing alginate lyase can be further crosslinked with bivalent Ca²⁺ cation by exposing beads to <10% w/v of calcium chloride for a duration of >1 mins to <24 hours. The duration of crosslinking period controls the degradation of alginate particles under physiological conditions.

Clause 307. The alginate microsphere of clause 306, wherein the calcium chloride solution further contains excipients required for the freeze-drying steps.

Clause 308. The alginate microsphere of clause 307, where in the Ca²⁺-crosslinked alginate beads containing alginate lyase are further washed in aqueous medium containing excipients to remove unbound Ca²⁺ ion.

Clause 309. The alginate microsphere of clause 308, wherein the Ca²⁺-crosslinked alginate beads containing alginate lyase are dispersed in a solution containing excipients and subjected to freeze drying to produce freeze dried Ca²⁺ crosslinked alginate beads containing alginate lyase enzyme.

Clause 310. The alginate microsphere of clause 309, wherein the freeze dried Ca²⁺ crosslinked alginate beads containing alginate lyase enzyme are further subjected to sterilization step (gamma-radiation and e-beam radiation).

Clause 311. The alginate microsphere of clause 310, wherein the freeze dried and sterilized Ca²⁺-crosslinked alginate beads containing alginate lyase enzyme can be reconstituted in aqueous solution of neutral pH which activates the alginate lyase enzyme and initiates the degradation of alginate particles.

Clause 401. An alginate microsphere capable of self-degradation upon rehydration, comprising:
an alginate lyase enzyme pre-treated by varying temperature, by varying pH, and/or with a metal-ion enzyme inhibitor;
alginate molecules having one or both of (i) a predetermined molecular weight, and (ii) a predetermined ratio of β-D-Mannuronic acid (M) blocks to α-L-Guluronic acid (G) blocks; and
a divalent metal-ion crosslinking the alginate molecules,
wherein the alginate microsphere is substantially free of water and/or sterilized.

Clause 402. The alginate microsphere of clause 401, wherein the degradation of alginate microsphere is controlled by one or more of the pre-treatment of the alginate lyase enzyme, an amount of the alginate enzyme in the microsphere, the predetermined molecular weight of the alginate molecule, and the predetermined ratio of M:G blocks of the alginate molecules, and a composition of gelling bath, including an amount and or charge of one or more ions in the gelling bath.

Clause 403. The alginate microsphere of clause 401, wherein at least one of (i)-(iii)
(i) the metal-ion enzyme inhibitor is a reversible inhibitor selected from the group consisting of Cu²⁺, Zn²⁺, and Fe³⁺,
(ii) the pre-treatment of the alginate enzyme in the precursor solution allows mixing of a predetermined amount of enzyme (measured in units, U) with the alginate molecules, and
(iii) an activity of the alginate lyase enzyme is modulated by adjusting one or more of a pH of a gelling bath, a temperature of the gelling bath, and an amount of the metal-ion enzyme inhibitor in the alginate microsphere.

Clause 404. The alginate microsphere of clause 401, wherein at least one of (i)-(v) applies:
(i) the predetermined molecular weight of the alginate molecules is in a range of greater than about 100 kDa to less than about 800 kDa,
(ii) the predetermined ratio of M:G blocks is about 50:50, about 55:45, about 60:40, about 65:35, about 70:30, about 75:25, about 80:20, about 85:15, about 90:10, or about 95:5,
(iii) the predetermined ratio of M:G blocks is about 50:50, about 45:55, about 40:60, about 35:65, about 30:70, about 25:75, about 20:80, about 15:85, about 10:90, or about 5:95,
(iv) an activity of the alginate lyase enzyme is between about 0.05 mU (milliunits) and about 2.5 mU per microsphere, and
(v) an activity of the alginate lyase enzyme is between about 0.05 nU (nanounits) and about 0.05 mU per microsphere.

Clause 405. The alginate microsphere of clause 404, wherein at least one of (a)-(d) applies:
(a) the alginate microsphere of (ii) degrades over a period of less than about 5 days or greater than about 2 days,
(b) the alginate microsphere of (iii) degrades over a period of between about 5 days and about 30 days,
(c) the alginate microsphere of (iv) degrades over a period of less than about 5 days, and
(d) the alginate microsphere of (v) degrades over a period of between about 5 days and about 30 days.

Clause 406. The alginate microsphere of clause 401, wherein at least one of (i)-(vii)
(i) the microsphere further comprises a bioactive agent,
(ii) the microsphere further comprises a cryoprotectant selected from the group consisting of hydroxypropyl-β cyclodextrin, trehalose, polyvinyl pyrrolidone of 40 kDa (PVP 40 kDa), dextran (70 kDa molecular weight), glucose, lactose, maltodextrins, mannitol, glycols, and polyglycols,
(iii) the alginate microsphere is lyophilized,
(iv) a sphericity of the alginate microsphere is at least about 0.7, at least about 0.75, at least about 0.8, at least about 0.85, at least about 0.9, at least about 0.95, or at least about 0.99,
(v) the alginate microsphere is sterilized, or the alginate microsphere is lyophilized and sterilized,
(vi) a shelf-life of the alginate microsphere is at least about 3 months, at least about 6 months, at least about 12 months, at least about 18 months, at least about 24 months, at least about 36 months, at least about 48 months, or at least about 60 months when stored at a given temperature, and
(vii) a lyophilized alginate microsphere is reconstituted in saline or saline-radiopaque contrast at physiological pH.

Clause 407. The alginate microsphere of clause 406, wherein at least one of (a)-(d) applies:
(a) the bioactive agent of (i) comprises an anti-inflammatory agent, an anesthetic drug, an anti-cancer agent, or an anti-angiogenic agent,
(b) a residual water content of the lyophilized alginate microsphere of (iii) is in the range of about 1% to about 3% by mass,
(c) the sterilization of (v) comprises high energy radiation sterilization, gamma-ray sterilization, or e-beam sterilization, and
(d) the given temperature of (vi) is between about 2° C. and about 8° C. or about room temperature (RT).

Clause 408. The alginate microsphere of clause 407, wherein the anti-inflammatory agent of (a) comprises hyaluronic acid having a molecular weight of between about 1 million (M) and about 5 M Daltons or the sterilization of (c) comprises between about 15 and about 25 kGy of gamma radiation from Cobalt 60 Isotope, or about 25 kGy of electron beam radiation in accordance with ISO 11137-1:2006.

Clause 409. A method of preparing an alginate microsphere capable of self-degradation upon rehydration, the method comprising:
forming droplets from a precursor solution, the precursor solution comprising:
an alginate lyase enzyme pre-treated by varying temperature, by varying pH, and/or with a metal-ion enzyme inhibitor; and
alginate molecules having one or both of (a) a predetermined molecular weight, and (b) a predetermined ratio of β-D-Mannuronic acid (M) blocks to α-L-Guluronic acid (G) blocks;
contacting the droplets with a gelling bath comprising a divalent metal-ion, thereby crosslinking the alginate molecules to form an alginate microsphere; and
dehydrating, and optionally sterilizing, the alginate microsphere thereby substantially removing water from the microsphere.

Clause 410. The method of clause 409, wherein at least one of (i)-(x) applies:
(i) the precursor solution comprises one or more cryoprotectants,
(ii) the gelling bath comprises one or more cryoprotectants,
(iii) the pH of alginate lyase enzyme in the precursor solution containing alginate lyase and alginate is in the range of pH 3.0 to 6.4,
(iv) the metal-ion enzyme inhibitor is a reversible inhibitor selected from the group consisting of Cu²⁺, Zn²⁺, and Fe³⁺,
(v) the temperature of the precursor solution is in the range of 1-4° C.,
(vi) the pre-treatment of the alginate enzyme in the precursor solution allows mixing of a predetermined amount of enzyme (measured in units, U) with the alginate molecules,
(vii) an activity of the alginate lyase enzyme is modulated by adjusting one or more of a pH of the gelling bath, a temperature of the gelling bath, and an amount of the metal-ion enzyme inhibitor in the alginate microsphere,
(viii) a pH of the gelling bath is less than about 6.5,
(ix) a pH of the gelling bath is equal to or about equal to a pH of the precursor solution, and
(x) the precursor solution and/or the gelling bath further comprises a bioactive agent.

Clause 411. The method of clause 409, wherein at least one of (i)-(iv) applies:
(i) the dehydrating comprises lyophilizing the alginate microsphere,
(ii) forming the droplets is performed using a method selected from the group consisting of drop casting, spray congealing/spray cooling, spray drying, microfluidic droplet production, and jet-cutting,
(iii) a sphericity of the alginate microsphere is at least about 0.7, at least about 0.75, at least about 0.8, at least about 0.85, at least about 0.9, at least about 0.95, or at least about 0.99, and
(iv) a shelf-life of the alginate microsphere is at least about 3 months, at least about 6 months, at least about 12 months, at least about 18 months, at least about 24 months, at least about 36 months, at least about 48 months, or at least about 60 months when stored at a given temperature.

Clause 412. The method of clause 410, wherein the cryoprotectant of (i) and (ii) is each independently selected from the group consisting of hydroxypropyl-β cyclodextrin, trehalose, polyvinyl pyrrolidone of 40 kDa (PVP 40 kDa), dextran (70 kDa molecular weight), glucose, lactose, maltodextrins, mannitol, glycols, and polyglycols.

Clause 413. The method of clause 412, wherein at least one of (a)-(f) applies:
(a) the concentration of the trehalose in the precursor solution (i) is about 0.1% w/v to about 20% w/v,
(b) the concentration of the hydroxypropyl-β cyclodextrin the precursor solution (i) or the gelling bath (ii) is about 0.1% w/v to about 2% w/v,
(c) the concentration of the PVP 40 kDa in the precursor solution (i) is about 0.1% w/v to about 1% w/v,
(d) the concentration of the dextran (molecular weight 70 kDa) in the precursor solution (i) is about 0.1% w/v to about 1% w/v,
(e) the precursor solution (i) and the gelling bath (ii) comprise the same cryoprotectant, and

(f) the precursor solution (i) and the gelling bath (ii) comprise the same cryoprotectant at equal or about equal concentrations.

Clause 414. The method of clause 411, wherein a residual water content of the lyophilized alginate microsphere is in the range of about 1% to about 3% by mass.

Clause 415. The method of clause 409, further comprising at least one step selected from (i)-(iv)
(i) sterilizing the alginate microsphere or the alginate microsphere that has been dehydrated by lyophilization,
(ii) storing the alginate microsphere for at least about 3 months, at least about 6 months, at least about 12 months, at least about 18 months, at least about 24 months, at least about 36 months, at least about 48 months, or at least about 60 months when stored at a given temperature,
(iii) administering the alginate microsphere, or the alginate microsphere that has been dehydrated by lyophilization, to a subject, and
(iv) reconstituting the alginate microsphere, or the alginate microsphere that has been dehydrated by lyophilization, using saline or saline-radiopaque contrast at physiological pH.

Clause 416. The method of clause 415, wherein at least one of (a)-(d) applies:
(a) the sterilizing of (i) comprises high energy radiation sterilization, gamma-ray sterilization, or e-beam sterilization,
(b) the sterilizing of (i) comprises between about 15 and about 25 kGy of gamma radiation from Cobalt 60 Isotope, or about 25 kGy of electron beam radiation in accordance with ISO 11137-1:2006,
(c) the given temperature of (ii) is between about 2° C. and about 8° C., and
(d) the given temperature of (ii) is about room temperature (RT).

Clause 417. The method of clause 409, wherein the degradation of alginate microsphere is controlled by one or more of the pre-treatment of the alginate lyase enzyme, an amount of the alginate enzyme in the microsphere, the predetermined molecular weight of the alginate molecule, and the predetermined ratio of M:G blocks of the alginate molecules, and a composition of gelling bath, including an amount and or charge of one or more ions in the gelling bath.

Clause 418. The method of clause 409, wherein at least one of (i)-(vii) applies:
(i) the predetermined molecular weight of the alginate molecules is in a range of greater than about 100 kDa to less than about 800 kDa,
(ii) the predetermined ratio of M:G blocks is about 50:50, about 55:45, about 60:40, about 65:35, about 70:30, about 75:25, about 80:20, about 85:15, about 90:10, or about 95:5, (iii) the predetermined ratio of M:G blocks is about 50:50, about 45:55, about 40:60, about 35:65, about 30:70, about 25:75, about 20:80, about 15:85, about 10:90, or about 5:95,
(iv) the pre-treated alginate lyase enzyme is mixed in the precursor solution having enzyme activity ranging from 0.0025 U/mg to 1 U/mg of alginate,
(v) the pre-treated alginate lyase enzyme is mixed in the precursor solution having enzyme activity ranging from 0.125 U/mg to 0.250 U/mg of alginate,
(vi) the pre-treated alginate lyase enzyme is mixed in the precursor solution having enzyme activity ranging from 0.025 U/mg to 0.125 U/mg of alginate, and
(vii) the pre-treated alginate lyase enzyme is mixed in the precursor solution having enzyme activity ranging from 0.0025 U/mg to 0.005 U/mg of alginate.

Clause 419. The method of clause 418, wherein at least one of (a)-(e) applies:
(a) the alginate microsphere degrades over a period of less than about 5 days or greater than about 2 days,
(b) the alginate microsphere of (iii) degrades over a period of between about 5 days and about 30 days,
(c) the alginate microsphere of (v) degrades over a period of less than about 5 days,
(d) the alginate microsphere of (vi) degrades over a period of between about 5 days and about 30 days, and
(e) the alginate microsphere of (vii) degrades over a period of greater than about 30 days.

Clause 420. The method of clause 410, wherein the bioactive agent of (x) comprises an anti-inflammatory agent, an anesthetic agent, anti-cancer agent, or an anti-angiogenic agent.

Clause 421. The method of clause 420, wherein the anti-inflammatory agent comprises hyaluronic acid having a molecular weight of between about 1 million (M) and about 5 M Daltons.

Clause 509. A method of preparing an alginate microsphere capable of self-degradation upon rehydration, the method comprising:
forming droplets from a precursor solution using a microfluidics platform, the precursor solution comprising:
   (i) an alginate lyase enzyme pre-treated with an alkaline pH and with a temperature less than about 15° C.;
   (ii) alginate molecules having one or both of (a) a predetermined molecular weight, and (b) a predetermined ratio of β-D-Mannuronic acid (M) blocks to α-L-Guluronic acid (G) blocks; and
   (iii) a bivalent cation crosslinking agent;
contacting the droplets with a gelling solution comprising oil and an acid, thereby crosslinking the alginate molecules to form an alginate microsphere; and
dehydrating, and optionally sterilizing, the alginate microsphere thereby substantially removing water from the microsphere.

Clause 510. The method of clause 509, wherein the degradation of alginate microsphere is controlled by one or more of the pre-treatment of the alginate lyase enzyme, an amount of the alginate enzyme in the microsphere, the predetermined molecular weight of the alginate molecule, and the predetermined ratio of M:G blocks of the alginate molecules, and bivalent cation crosslinking of the alginate molecules.

Clause 511. The method of clause 509 or 510, wherein at least one of (i)-(viii) applies:
(i) the alginate lyase enzyme is pre-treated with a pH of about 8 to about 13,
(ii) the alginate lyase enzyme is pre-treated with a temperature of about 1° C. to about 4° C.,
(iii) the pre-treatment of the alginate enzyme in the precursor solution allows mixing of a predetermined amount of enzyme (measured in units, U) with the alginate molecules,
(iv) the precursor solution has a pH of about 8 to about 13 and is maintained at a temperature of about 1° C. to about 4° C.,
(v) the precursor solution further comprises an excipient,
(vi) the bivalent cation crosslinking agent is Ca²⁺ released from Ca-EDTA or CaCO₃,
(vii) the acid is acetic acid, and
(viii) the gelling solution further comprises a surfactant.

Clause 512. The method of any one of clauses 509-511, wherein the gelling solution comprises oil, about 0.05% v/v to about 5% v/v acetic acid, and a surfactant and the droplets are contacted with the gelling solution for about 1 minute to about 3 hours, crosslinking the alginate molecules.

Clause 513. The method of any one of clauses 509-512, wherein the step of dehydrating, and optionally sterilizing, the alginate microsphere is preceded by crosslinking the alginate microspheres with a bivalent Ca²⁺ ion.

Clause 514. The method of any one of clauses 509-512, wherein the step of dehydrating, and optionally sterilizing, the alginate microsphere is preceded by further crosslinking the alginate molecules with a second bivalent Ca²⁺ ion.

Clause 515. The method of clause 514, wherein further crosslinking the alginate molecules with a second bivalent Ca²⁺ ion forms alginate microspheres crosslinked with a bivalent Ca²⁺ ion.

Clause 516. The method of clause 513 or 514, wherein the alginate microspheres or alginate molecules are crosslinked by exposure to a solution comprising less than about 10% w/v CaCl₂) for about 1 minute to about 24 hours.

Clause 517. The method of clause 516, wherein the solution further comprises an excipient.

Clause 518. The method of clause 517, wherein the step of dehydrating the alginate microsphere or the bivalent Ca²⁺ crosslinked alginate microspheres comprises lyophilizing the alginate microspheres or the bivalent Ca²⁺ crosslinked alginate microspheres.

Clause 519. The method of clause 518, comprising sterilizing the lyophilized alginate microsphere or the lyophilized bivalent Ca²⁺ crosslinked alginate microspheres using gamma radiation and/or e-beam radiation.

Clause 520. The method of clause 519, comprising reconstituting the lyophilized and sterilized alginate microspheres or the lyophilized and sterilized bivalent Ca²⁺ crosslinked alginate microspheres in an aqueous solution of neutral pH.

Clause 601. A microsphere capable of self-degradation upon rehydration, comprising:
an enzyme pre-treated by varying temperature, by varying pH, and/or with a metal-ion enzyme inhibitor; and
a crosslinked biomaterial;
   wherein:
the crosslinked biomaterial forms a biologically derived microsphere encapsulating the enzyme; and
the microsphere is substantially free of water and/or sterilized.

Clause 602. The microsphere of clause 601, wherein the enzyme is an enzyme that acts on the biomaterial, degrading the biomaterial.

Clause 603. The microsphere of clause 601 or 602, wherein the biomaterial comprises a polysaccharide, a protein, or a glycoprotein.

Clause 604. The microsphere of any one of clauses 601-603, further comprising a photoinitiator and wherein the biomaterial comprises a photo-crosslinkable moiety which is photo-crosslinked.

Clause 605. The microsphere of any one of clauses 601-604, wherein the self-degradation of the microsphere is controlled by one or more of: the pre-treatment of the enzyme, the concentration of the enzyme in the microsphere, the activity the enzyme, and the predetermined molecular weight of the biomaterial.

Clause 606. The microsphere of any one of clauses 601-605, wherein the biomaterial is crosslinked by a divalent metal ion.

Clause 607. The microsphere of clause 606, wherein the self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial.

Clause 608. The microsphere of any one of clauses 601-607, wherein the microsphere self-degrades in less than about 4 hours, over a period of greater than about 2 days to less than about 5 days, over a period of greater than about 5 days to less than about 30 days, or greater than about 30 days.

Clause 609. The microsphere of any one of clauses 601-608, wherein:
the enzyme activity is between about 0.0075 U/mg to 0.25 U/mg of the biomaterial and the microsphere degrades over a period of greater than about 20 minutes to less than about 4 hours;
the enzyme activity is between about 0.005 U/mg to about ≥0.0025 U/mg of the biomaterial and the microsphere degrades over a period of greater than about 5 days to less than about 30 days; or the enzyme activity is less than about 0.0025 U/mg of the biomaterial, and the microsphere degrades over a period of greater than about 30 days.

Clause 610. The microsphere of any one of clauses 601-609, wherein the residual water content of the microsphere is between about 1% by mass and about 10% by mass.

Clause 611. The microsphere of clause 610, wherein the microsphere is lyophilized or dehydrated using super critical CO₂.

Clause 612. The microsphere of any one of clauses 601-611, wherein the microsphere is sterilized with about 6-10 kGy of gamma radiation.

Clause 613. The microsphere of any one of clauses 601-612, wherein the microsphere further comprises an anti-inflammatory agent, a chemotherapeutic agent, an antioxidant, a corticosteroid, or a combination thereof.

Clause 614. The microsphere of clause 613, wherein the microsphere comprises one or more of:
an anti-inflammatory agent selected from hyaluronic acid, ibuprofen, flurbiprofen, meloxicam, nabumetone, etodolac, ketorolac, ketoprofen, diflusinal, naproxen, diclofenac, celecoxib, mefenamic acid, etoricoxib, indomethacin, aspirin, arnica, curcurmin, bromelain and acetaminophen,
an antioxidant selected from glutathione, a-tocopherol, ergothioneine, N-acetylcysteine, ascorbic acid, vitamin A, vitamin E, allopurinol, melatonin, resveratrol, bucillamine, turmeric, lycopene, dihydrolipoic acid, cobalamins, flavonoids, quercetin, ebselen, and edaravone; or
a corticosteroid selected from methylprednisolone, dexamethasone, triamcinolone, betamethasone, beclomethasone, and hydrocortisone.

Clause 615. The microsphere of any one of clauses 601-614, wherein: the biomaterial comprises alginate and the enzyme is alginate lyase, the biomaterial comprises pectin and the enzyme is pectinase, the biomaterial comprises hyaluronic acid and the enzyme is hyaluronidase, the biomaterial comprises gelatin and the enzyme is a matrix metalloproteinase or protease, the biomaterial comprises albumin and the enzyme is peptidase, the biomaterial comprises collagen and the enzyme is protease, the biomaterial comprises fibrinogen and the enzyme is plasmin, the biomaterial comprises silk fibrin and the enzyme is protease, the biomaterial comprises starch and the enzyme is amylase, the biomaterial comprises chitosan and the enzyme is chitosanase or lysozyme, the biomaterial comprises agar/agarose and the enzyme is agarase, the biomaterial comprises carrageenan and the enzyme is carrageenase, the biomaterial comprises pullulan and the enzyme is pullulanase, the biomaterial comprises dextran and the enzyme is dextranase, the biomaterial comprises b-glycan and the enzyme is b-glycanse, the biomaterial comprises cellulose and the enzyme is cellulase, or the biomaterial comprises lignin and the enzyme is ligninase.

Clause 616. The microsphere of clause 615, wherein the biomaterial comprises alginate and the enzyme is alginate lyase.

Clause 617. The microsphere of clause 616, wherein the enzyme is pre-treated by maintaining the enzyme at a pH of about 3.0 to about 6.4, by maintaining the enzyme at a temperature of about 1° C. to about 4° C., or by treating the enzyme with a reversible metal-ion enzyme inhibitor selected from Cu²⁺, Zn²⁺, and Fe³⁺.

Clause 618. The microsphere of clause 616 or 617, wherein the biomaterial is crosslinked by a divalent metal ion selected from Cu²⁺, Ba²⁺, Sr²⁺, Ca²⁺, Co²⁺, Ni²⁺, Mn²⁺ and Mg²⁺.

Clause 619. The microsphere of any one of clauses 616-618, wherein:
the enzyme activity is about 0.0075 U/mg to about 0.25 U/mg of alginate and the microsphere degrades over a period of less than about 4 hours;
the enzyme activity is about 0.005 U/mg to about 0.0025 U/mg of alginate and the microsphere degrades over a period of between about 5 days and about 30 days; or
the enzyme activity is less than about 0.0025 U/mg of the biomaterial and the microsphere degrades over a period of greater than about 30 days.

Clause 620. A method of preparing a microsphere capable of self-degradation upon rehydration, the method comprising:
forming droplets from a precursor solution, the precursor solution comprising:
   (i) an enzyme pre-treated by varying temperature, by varying pH, and/or with a metal-ion enzyme inhibitor; and
   (ii) a biomaterial;
contacting the droplets with a gelling bath comprising a cryoprotectant and a divalent metal ion, thereby cross-linking the biomaterial to form a biologically derived microsphere encapsulating the enzyme; and
dehydrating, and optionally sterilizing, the microsphere thereby substantially removing water from the microsphere.

Clause 621. The method of clause 620, wherein the precursor solution further comprises one or more cryoprotectants.

Clause 622. The method of clause 620 or 621, wherein each cryoprotectant is independently selected from hydroxypropyl-β-cyclodextrin, trehalose, polyvinyl pyrrolidone, and dextran.

Clause 623. The method of any one of clauses 620-622, wherein the enzyme is an enzyme that acts on the biomaterial, degrading the biomaterial.

Clause 624. The method of any one of clauses 620-623, wherein the biomaterial comprises a polysaccharide, a protein, or a glycoprotein.

Clause 625. The method of any one of clauses 620-624, wherein the microsphere comprises: alginate particles encapsulating alginate lyase, pectin particles encapsulating pectinase, gelatin particles encapsulating a matrix metalloproteinase or protease, or carrageenan particles encapsulating carrageenase.

Clause 626. The method of any one of clauses 620-625, wherein the self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial.

Clause 627. The method of any one of clauses 620-626, wherein the microsphere self-degrades in less than about 4 hours, over a period of greater than about 2 days to less than about 5 days, over a period of greater than about 5 days to less than about 30 days, or greater than about 30 days.

Clause 628. The method of any one of clauses 620-627, wherein:
the enzyme activity is between about 0.0075 U/mg to 0.25 U/mg of the biomaterial and the microsphere degrades over a period of greater than about 20 minutes to less than about 4 hours;
the enzyme activity is between about 0.005 U/mg to about ≥0.0025 U/mg of the biomaterial and the microsphere degrades over a period of greater than about 5 days to less than about 30 days; or the enzyme activity is less than about 0.0025 U/mg of the biomaterial, and the microsphere degrades over a period of greater than about 30 days.

Clause 629. The method of any one of clauses 620-628, wherein:
the droplets are contacted with the gelling bath for a range of 10 minutes to 1 hour and the resulting microspheres degrade over a period of greater than about 20 minutes to less than about 4 hours;
the droplets are contacted with the gelling bath for a range of 1 hour to 12 hours and the resulting microspheres degrade over a period of greater than about 5 days to less than about 30 days; or
the droplets are contacted with the gelling bath for a range of 12 hours to 24 hours and the resulting microspheres degrade over a period of greater than about 30 days.

Clause 630. The method of any one of clauses 620-629, wherein the residual water content of the microsphere is between about 1% by mass and about 10% by mass.

Clause 631. The method of any one of clauses 620-630, wherein dehydrating comprises lyophilizing the microsphere or drying the microsphere using super critical CO₂.

Clause 632. The method of any one of clauses 620-631, wherein the sterilizing comprises irradiating the microsphere with 6-10 kGy of gamma radiation.

Clause 633. The method of any one of clauses 620-632, wherein the precursor solution and/or the gelling bath further comprise an anti-inflammatory agent, a chemotherapeutic agent, an antioxidant, or a combination thereof.

Clause 634. The method of clause 633, wherein the microsphere comprises one or more of:
an anti-inflammatory agent selected from hyaluronic acid, ibuprofen, flurbiprofen, meloxicam, nabumetone, etodolac, ketorolac, ketoprofen, diflusinal, naproxen, diclofenac, celecoxib, mefenamic acid, etoricoxib, indomethacin, aspirin, arnica, curcumin, bromelain and acetaminophen,
an antioxidant selected from glutathione, a-tocopherol, ergothioneine, N-acetylcysteine, ascorbic acid, vitamin A, vitamin E, allopurinol, melatonin, resveratrol, bucillamine, turmeric, lycopene, dihydrolipoic acid, cobalamins, flavonoids, quercetin, ebselen, and edaravone; or
a corticosteroid selected from methylprednisolone, dexamethasone, triamcinolone, betamethasone, beclomethasone, and hydrocortisone.

Clause 635. A method of preparing a photopolymerized microsphere capable of self-degradation upon rehydration, the method comprising:
forming droplets from a precursor solution, the precursor solution comprising:
   (i) an enzyme pre-treated by varying temperature, by varying pH, and/or with a metal-ion enzyme inhibitor;
   (ii) a biomaterial comprising a photo-crosslinkable moiety;
   (iii) a photoinitiator;
irradiating the droplets, thereby cross-linking the biomaterial to form a photo-polymerized biologically derived microsphere encapsulating the enzyme; and
dehydrating, and optionally sterilizing, the microsphere thereby substantially removing water from the microsphere.

Clause 636. The method of clause 635, wherein the photo-crosslinkable moiety is selected from an acrylate group, a methacrylate group, a vinyl group, and an allyl group.

Clause 637. The method of clause 635 or 636, wherein the precursor solution further comprises one or more cryoprotectants.

Clause 638. The method of any one of clauses 635-637, wherein the cryoprotectant is selected from hydroxypropyl-β-cyclodextrin, trehalose, polyvinyl pyrrolidone, and dextran.

Clause 639. The method of any one of clauses 635-638, wherein the enzyme is an enzyme that acts on the biomaterial, degrading the biomaterial.

Clause 640. The method of any one of clauses 635-639, wherein the biomaterial comprises a polysaccharide, a protein, or a glycoprotein.

Clause 641. The method of any one of clauses 635-640, wherein the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase.

Clause 642. The method of any one of clauses 635-641, wherein the self-degradation of the microsphere is controlled by one or more of: the pre-treatment of the enzyme, the concentration of the enzyme in the microsphere, the enzyme activity, the predetermined molecular weight of the biomaterial, and the amount of time that the droplets are irradiated.

Clause 643. The method of any one of clauses 635-642, wherein the microsphere self-degrades in less than about 4 hours, over a period of greater than about 2 days to less than about 5 days, over a period of greater than about 5 days to less than about 30 days, or greater than about 30 days.

Clause 644. The method of any one of clauses 635-643, wherein:
the enzyme activity is between about 0.0075 U/mg to 0.25 U/mg of the biomaterial and the microsphere degrades over a period of greater than about 20 minutes to less than about 4 hours;
the enzyme activity is between about 0.005 U/mg to about ≥0.0025 U/mg of the biomaterial and the microsphere degrades over a period of greater than about 5 days to less than about 30 days; or
the enzyme activity is less than about 0.0025 U/mg of the biomaterial, and the microsphere degrades over a period of greater than about 30 days.

Clause 645. The method of any one of clauses 635-644, wherein the residual water content of the microsphere is between about 1% by mass and about 10% by mass.

Clause 646. The method of any one of clauses 635-645, wherein dehydrating comprises lyophilizing the microsphere or drying the microsphere using super critical CO₂.

Clause 647. The method of any one of clauses 635-646, wherein the sterilizing comprises irradiating the microsphere with 6-10 kGy of gamma radiation.

Clause 648. The method of any one of clauses 635-647, wherein the precursor solution further comprises an anti-inflammatory agent, a chemotherapeutic agent, an antioxidant, or a combination thereof.

Clause 649. The method of clause 648, wherein the microsphere comprises one or more of:
an anti-inflammatory agent selected from hyaluronic acid, ibuprofen, flurbiprofen, meloxicam, nabumetone, etodolac, ketorolac, ketoprofen, diflusinal, naproxen, diclofenac, celecoxib, mefenamic acid, etoricoxib, indomethacin, aspirin, arnica, curcurmin, bromelain and an antioxidant selected from glutathione, a-tocopherol, ergothioneine, N-acetylcysteine, ascorbic acid, vitamin A, vitamin E, allopurinol, melatonin, resveratrol, bucillamine, turmeric, lycopene, dihydrolipoic acid, cobalamins, flavonoids, quercetin, ebselen, and edaravone; or a corticosteroid selected from methylprednisolone, dexamethasone, triamcinolone, betamethasone, beclomethasone, and hydrocortisone.

Clause 650. A method of preparing a microsphere capable of self-degradation upon rehydration, the method comprising:
forming droplets from a precursor solution, the precursor solution comprising:
   (i) a biomaterial comprising a covalently crosslinkable moiety; and
   (ii) a homo-bifunctional crosslinking agent or a heterobifunctional crosslinking agent;
covalently cross-linking the biomaterial to form a biologically derived microsphere;
swelling an enzyme that has been pre-treated by varying temperature, by varying pH, and/or with a metal-ion enzyme inhibitor into the microsphere such that the biologically derived microsphere encapsulates the enzyme; and
dehydrating, and optionally sterilizing, the microsphere thereby substantially removing water from the microsphere.

Clause 651. The method of clause 650, wherein the covalently crosslinkable moiety comprises an amine group or a carboxyl group.

Clause 652. The method of clause 650 or 651, wherein the precursor solution further comprises one or more cryoprotectants.

Clause 653. The method of clause 652, wherein the cryoprotectant is selected from hydroxypropyl-β-cyclodextrin, trehalose, polyvinyl pyrrolidone, and dextran.

Clause 654. The method of any one of clauses 650-653, wherein the enzyme is an enzyme that acts on the biomaterial, degrading the biomaterial.

Clause 655. The method of any one of clauses 650-654, wherein the biomaterial comprises a polysaccharide, a protein, or a glycoprotein.

Clause 656. The method of any one of clauses 650-655, wherein the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase.

Clause 657. The method of any one of clauses 650-656, wherein the self-degradation of the microsphere is controlled by one or more of: the pre-treatment of the enzyme, the concentration of the enzyme in the microsphere, the enzyme activity, the predetermined molecular weight of the biomaterial, and the homo-bifunctional crosslinking agent or heterobifunctional crosslinking used to crosslink the biomaterial.

Clause 658. The method of any one of clauses 650-657, wherein the microsphere self-degrades in less than about 4 hours, over a period of greater than about 2 days to less than about 5 days, over a period of greater than about 5 days to less than about 30 days, or greater than about 30 days.

Clause 659. The method of any one of clauses 650-658, wherein:
the enzyme activity is between about 0.0075 U/mg to 0.25 U/mg of the biomaterial and the microsphere degrades over a period of greater than about 20 minutes to less than about 4 hours;
the enzyme activity is between about 0.005 U/mg to about ≥0.0025 U/mg of the biomaterial and the microsphere degrades over a period of greater than about 5 days to less than about 30 days; or
the enzyme activity is less than about 0.0025 U/mg of the biomaterial, and the microsphere degrades over a period of greater than about 30 days.

Clause 660. The method of any one of clauses 650-659, wherein the residual water content of the microsphere is between about 1% by mass and about 10% by mass.

Clause 661. The method of any one of clauses 650-660, wherein dehydrating comprises lyophilizing the microsphere or drying the microsphere using super critical CO₂.

Clause 662. The method of any one of clauses 650-661, wherein the sterilizing comprises irradiating the microsphere with 6-10 kGy of gamma radiation.

Clause 663. The method of any one of clauses 650-662, wherein the precursor solution further comprises an anti-inflammatory agent, a chemotherapeutic agent, an antioxidant, or a combination thereof.

Clause 664. The method of clause 663, wherein the microsphere comprises one or more of:
an anti-inflammatory agent selected from hyaluronic acid, ibuprofen, flurbiprofen, meloxicam, nabumetone, etodolac, ketorolac, ketoprofen, diflusinal, naproxen, diclofenac, celecoxib, mefenamic acid, etoricoxib, indomethacin, aspirin, arnica, curcurmin, bromelain and acetaminophen,
an antioxidant selected from glutathione, a-tocopherol, ergothioneine, N-acetylcysteine, ascorbic acid, vitamin A, vitamin E, allopurinol, melatonin, resveratrol, bucillamine, turmeric, lycopene, dihydrolipoic acid, cobalamins, flavonoids, quercetin, ebselen, and edaravone; or
a corticosteroid selected from methylprednisolone, dexamethasone, triamcinolone, betamethasone, beclomethasone, and hydrocortisone.

Clause 665. A method of preparing a microsphere capable of self-degradation upon rehydration, the method comprising:
forming droplets from a precursor solution, the precursor solution comprising:
   (i) a biomaterial comprising a covalently crosslinkable moiety;
   (ii) an enzyme that has been pre-treated by varying temperature, by varying pH, and/or with a metal-ion enzyme inhibitor; and
   (iii) a homo-bifunctional crosslinking agent or a heterobifunctional crosslinking agent;
covalently cross-linking the biomaterial to form a biologically derived microsphere encapsulating the enzyme; and
dehydrating, and optionally sterilizing, the microsphere thereby substantially removing water from the microsphere.

Clause 666. The method of clause 665, wherein the covalently crosslinkable moiety comprises an amine group or a carboxyl group.

Clause 667. The method of clause 665 or 666, wherein the precursor solution further comprises one or more cryoprotectants.

Clause 668. The method of clause 667, wherein the cryoprotectant is selected from hydroxypropyl-β-cyclodextrin, trehalose, polyvinyl pyrrolidone, and dextran.

Clause 669. The method of any one of clauses 665-668, wherein the enzyme is an enzyme that acts on the biomaterial, degrading the biomaterial.

Clause 670. The method of any one of clauses 665-669, wherein the biomaterial comprises a polysaccharide, a protein, or a glycoprotein.

Clause 671. The method of any one of clauses 665-670, wherein the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase.

Clause 672. The method of any one of clauses 665-671, wherein the self-degradation of the microsphere is controlled by one or more of: the pre-treatment of the enzyme, the concentration of the enzyme in the microsphere, the enzyme activity, the predetermined molecular weight of the biomaterial, and the homo-bifunctional crosslinking agent or heterobifunctional crosslinking used to crosslink the biomaterial.

Clause 673. The method of any one of clauses 665-672, wherein the microsphere self-degrades in less than about 4 hours, over a period of greater than about 2 days to less than about 5 days, over a period of greater than about 5 days to less than about 30 days, or greater than about 30 days.

Clause 674. The method of any one of clauses 665-673, wherein:
the enzyme activity is between about 0.0075 U/mg to 0.25 U/mg of the biomaterial and the microsphere degrades over a period of greater than about 20 minutes to less than about 4 hours;
the enzyme activity is between about 0.005 U/mg to about ≥0.0025 U/mg of the biomaterial and the microsphere degrades over a period of greater than about 5 days to less than about 30 days; or
the enzyme activity is less than about 0.0025 U/mg of the biomaterial, and the microsphere degrades over a period of greater than about 30 days.

Clause 675. The method of any one of clauses 665-674, wherein the residual water content of the microsphere is between about 1% by mass and about 10% by mass.

Clause 676. The method of any one of clauses 665-675, wherein dehydrating comprises lyophilizing the microsphere or drying the microsphere using super critical CO₂.

Clause 677. The method of any one of clauses 665-676, wherein the sterilizing comprises irradiating the microsphere with 6-10 kGy of gamma radiation.

Clause 678. The method of any one of clauses 665-677, wherein the precursor solution further comprises an anti-inflammatory agent, a chemotherapeutic agent, an antioxidant, or a combination thereof.

Clause 679. The method of clause 678, wherein the microsphere comprises one or more of:
an anti-inflammatory agent selected from hyaluronic acid, ibuprofen, flurbiprofen, meloxicam, nabumetone, etodolac, ketorolac, ketoprofen, diflusinal, naproxen, diclofenac, celecoxib, mefenamic acid, etoricoxib, indomethacin, aspirin, arnica, curcurmin, bromelain and acetaminophen,
an antioxidant selected from glutathione, a-tocopherol, ergothioneine, N-acetylcysteine, ascorbic acid, vitamin A, vitamin E, allopurinol, melatonin, resveratrol, bucillamine, turmeric, lycopene, dihydrolipoic acid, cobalamins, flavonoids, quercetin, ebselen, and edaravone; or
a corticosteroid selected from methylprednisolone, dexamethasone, triamcinolone, betamethasone, beclomethasone, and hydrocortisone.

Clause 680. A method of preparing a thermogelated microsphere capable of self-degradation upon rehydration, the method comprising:
heating a precursor solution comprising a biomaterial to melt the biomaterial;
adding to the precursor solution an enzyme pre-treated by varying temperature, by varying pH, and/or with a metal-ion enzyme inhibitor;
forming droplets from the precursor solution;
cooling the droplets to form a thermogelated biologically derived microsphere encapsulating the enzyme; and
dehydrating, and optionally sterilizing, the microsphere thereby substantially removing water from the microsphere.

Clause 681. The method of clause 680, wherein the precursor solution further comprises one or more cryoprotectants.

Clause 682. The method of clause 681, wherein the cryoprotectant is selected from hydroxypropyl-β-cyclodextrin, trehalose, polyvinyl pyrrolidone, and dextran.

Clause 683. The method of any one of clauses 680-682, wherein the enzyme is an enzyme that acts on the biomaterial, degrading the biomaterial.

Clause 684. The method of any one of clauses 680-683, wherein the biomaterial comprises a polysaccharide, a protein, or a glycoprotein.

Clause 685. The method of any one of clauses 680-684, wherein the microsphere comprises pectin particles encapsulating pectinase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, or agar/agarose particles encapsulating agarose.

Clause 686. The method of any one of clauses 680-685, wherein the self-degradation of the microsphere is controlled by one or more of: the pre-treatment of the enzyme, the concentration of the enzyme in the microsphere, the enzyme activity, and the predetermined molecular weight of the biomaterial.

Clause 687. The method of any one of clauses 680-686, wherein the microsphere self-degrades in less than about 4 hours, over a period of greater than about 2 days to less than about 5 days, over a period of greater than about 5 days to less than about 30 days, or greater than about 30 days.

Clause 688. The method of any one of clauses 680-687, wherein:
the enzyme activity is between about 0.0075 U/mg to 0.25 U/mg of the biomaterial and the microsphere degrades over a period of greater than about 20 minutes to less than about 4 hours;
the enzyme activity is between about 0.005 U/mg to about ≥0.0025 U/mg of the biomaterial and the microsphere degrades over a period of greater than about 5 days to less than about 30 days; or
the enzyme activity is less than about 0.0025 U/mg of the biomaterial, and the microsphere degrades over a period of greater than about 30 days.

Clause 689. The method of any one of clauses 680-688, wherein the residual water content of the microsphere is between about 1% by mass and about 10% by mass.

Clause 690. The method of any one of clauses 680-689, wherein dehydrating comprises lyophilizing the microsphere or drying the microsphere using super critical CO₂.

Clause 691. The method of any one of clauses 680-690, wherein the sterilizing comprises irradiating the microsphere with 6-10 kGy of gamma radiation.

Clause 692. The method of any one of clauses 680-691, wherein the precursor solution further comprises an anti-inflammatory agent, a chemotherapeutic agent, an antioxidant, or a combination thereof.

Clause 693. The method of clause 692, wherein the microsphere comprises one or more of:
an anti-inflammatory agent selected from hyaluronic acid, ibuprofen, flurbiprofen, meloxicam, nabumetone, etodolac, ketorolac, ketoprofen, diflusinal, naproxen, diclofenac, celecoxib, mefenamic acid, etoricoxib, indomethacin, aspirin, arnica, curcurmin, bromelain and acetaminophen,
an antioxidant selected from glutathione, a-tocopherol, ergothioneine, N-acetylcysteine, ascorbic acid, vitamin A, vitamin E, allopurinol, melatonin, resveratrol, bucillamine, turmeric, lycopene, dihydrolipoic acid, cobalamins, flavonoids, quercetin, ebselen, and edaravone; or
a corticosteroid selected from methylprednisolone, dexamethasone, triamcinolone, betamethasone, beclomethasone, and hydrocortisone.

Clause 694. A method of preparing a microsphere capable of self-degradation, the method comprising:
forming a precursor solution, the precursor solution comprising:
   (i) an enzyme; and
   (ii) a biomaterial;
passing the precursor solution through a needle under the influence of an electrostatic potential, forming droplets; and
contacting the droplets with a gelling bath comprising a divalent metal ion, thereby cross-linking the biomaterial to form a biologically derived microsphere encapsulating the enzyme.

Clause 695. The method of claim 694, further comprising dehydrating, and optionally sterilizing, the microsphere thereby substantially removing water from the microsphere to form a microsphere capable of self-degradation upon rehydration.

Clause 696. The method of claim 694 or 695, wherein the precursor solution further comprises one or more cryoprotectants.

Clause 697. The method of claim 696, wherein the cryoprotectant is selected from hydroxypropyl-β-cyclodextrin, trehalose, polyvinyl pyrrolidone, and dextran.

Clause 698. The method of any one of claims 694-697, wherein the electrostatic potential is between about 1 kV and about 5 kV.

Clause 699. The method of any one of claims 694-698, wherein the enzyme is an enzyme that acts on the biomaterial, degrading the biomaterial.

Clause 700. The method of any one of claims 694-699, wherein the biomaterial comprises a polysaccharide, a protein, or a glycoprotein.

Clause 701. The method of any one of claims 694-700, wherein the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase.

Clause 702. The method of any one of claims 694-701, wherein self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial.

Clause 703. The method of any one of claims 694-702, wherein the microsphere self-degrades in less than about 4 hours, over a period of greater than about 2 days to less than about 5 days, over a period of greater than about 5 days to less than about 30 days, or greater than about 30 days.

Clause 704. The method of any one of claims 694-703, wherein:
the enzyme activity is between about 0.0075 U/mg to 0.25 U/mg of the biomaterial and the microsphere degrades over a period of greater than about 20 minutes to less than about 4 hours;
the enzyme activity is between about 0.005 U/mg to about ≥0.0025 U/mg of the biomaterial and the microsphere degrades over a period of greater than about 5 days to less than about 30 days; or
the enzyme activity is less than about 0.0025 U/mg of the biomaterial, and the microsphere degrades over a period of greater than about 30 days.

Clause 705. The method of any one of claims 694-704, wherein the residual water content of the microsphere is between about 1% by mass and about 10% by mass.

Clause 706. The method of any one of claims 695-705, wherein dehydrating comprises lyophilizing the microsphere or drying the microsphere using super critical CO₂.

Clause 707. The method of any one of claims 695-706, wherein the sterilizing comprises irradiating the microsphere with 6-10 kGy of gamma radiation.

Clause 708. The method of any one of claims 694-707, wherein the precursor solution further comprises an anti-inflammatory agent, a chemotherapeutic agent, an antioxidant, or a combination thereof.

Clause 709. The method of claim 708, wherein the microsphere comprises one or more of:
an anti-inflammatory agent selected from hyaluronic acid, ibuprofen, flurbiprofen, meloxicam, nabumetone, etodolac, ketorolac, ketoprofen, diflusinal, naproxen, diclofenac, celecoxib, mefenamic acid, etoricoxib, indomethacin, aspirin, arnica, curcurmin, bromelain and
an antioxidant selected from glutathione, a-tocopherol, ergothioneine, N-acetylcysteine, ascorbic acid, vitamin A, vitamin E, allopurinol, melatonin, resveratrol, bucillamine, turmeric, lycopene, dihydrolipoic acid, cobalamins, flavonoids, quercetin, ebselen, and edaravone; or
a corticosteroid selected from methylprednisolone, dexamethasone, triamcinolone, betamethasone, beclomethasone, and hydrocortisone.

Clause 694. A method of inducing a self-degrading embolism in a subject in need thereof, comprising administering a plurality of the microspheres of any one of clauses 601-619 into a blood vessel of the subject.

Clause 695. The method of clause 694, wherein the blood vessel is a geniculate artery.

Clause 696. The method of clause 694 or 695, wherein the method induces a prostate arterial embolism, induces a uterine artery embolism, or the microsphere comprises a chemotherapeutic agent or is mixed with a chemotherapeutic agent and the method induces a transarterial chemoembolism (TACE).

Clause 697. A method of treating a disease or disorder in a subject in need thereof, comprising administering to the subject a plurality of the microspheres of any one of clauses 601-619.

Clause 698. The method of clause 697, wherein the disease or disorder is tendinopathy.

Clause 699. The method of clause 697, wherein the disease or disorder is selected from osteoarthritis, frozen shoulder, tennis elbow (lateral epicondylitis), golfer's elbow (medial epicondylitis), pitcher's elbow (flexor tendinitis), Achilles tendinopathy, plantar fasciitis, symptomatic accessory navicular bone, hamstring tendinopathy, jumper's knee (patellar tendonitis), runner's knee (patellofemoral pain syndrome (PFPS)), pes anserine bursitis (knee pain), posterior tibial muscle tendinopathy, wrist (TFCC-Triangular FibroCartilage Complex) tendinopathy, trigger finger (stenosing flexor tenosynovitis), and haemarthrosis.

Clause 700. A method of rapidly degrading a microsphere in a subject, comprising administering to the subject a bail out solution, wherein a plurality of microspheres of any one of clauses 601-619 was previously administered to the subject and the bail out solution comprises an enzyme capable of degrading the microspheres.

Clause 701. The method of clause 700, wherein the enzyme is complementary to the biomaterial used to form the plurality of microspheres.

Clause 702. The method of clause 700 or 701, wherein:
the microsphere comprises alginate particles encapsulating alginate lyase and the enzyme is alginate lyase,
the microsphere comprises pectin particles encapsulating pectinase and the enzyme is pectinase,
the microsphere comprises hyaluronic acid particles encapsulating hyaluronidase and the enzyme is hyaluronidase,
the microsphere comprises gelatin particles encapsulating a matrix metalloproteinase or protease and the enzyme is a matrix metalloproteinase or protease,
the microsphere comprises albumin particles encapsulating peptidase and the enzyme is peptidase,
the microsphere comprises collagen particles encapsulating protease and the enzyme is protease,
the microsphere comprises fibrinogen particles encapsulating plasmin and the enzyme is plasmin,
the microsphere comprises silk fibrin particles encapsulating protease and the enzyme is protease,
the microsphere comprises starch particles encapsulating amylase and the enzyme is amylase,
the microsphere comprises chitosan particles encapsulating chitosanase or lysozyme and the enzyme is chitosanase or lysozyme,
the microsphere comprises agar/agarose particles encapsulating agarase and the enzyme is agarase,
the microsphere comprises carrageenan particles encapsulating carrageenase and the enzyme is carrageenase,
the microsphere comprises pullulan particles encapsulating pullulanase and the enzyme is pullulanase,
the microsphere comprises dextran particles encapsulating dextranase and the enzyme is dextranase,
the microsphere comprises b-glycan particles encapsulating b-glycanase and the enzyme is b-glycanase,
the microsphere comprises cellulose particles encapsulating cellulase and the enzyme is cellulase, or
the microsphere comprises lignin particles encapsulating ligninase and the enzyme is ligninase.

Clause 703. The method of any one of clauses 700-702, wherein the bail out solution further comprises a divalent metal chelator.

Clause 704. A method of rapidly degrading a divalent metal ion crosslinked microsphere in a subject, comprising administering to the subject a bail out solution, wherein a plurality of divalent metal ion crosslinked microspheres of any one of clauses 601-619 was previously administered to the subject and the bail out solution comprises an anion, a phosphate buffer, or a combination thereof.

Clause 705. The method of clause 704, wherein the anion comprises citrate.

Clause 706. The method of clause 704, wherein the phosphate buffer comprises phosphate buffered saline.

Clause 707. The method of any one of clauses 704-706, wherein the microsphere comprises alginate particles encapsulating alginate lyase, pectin particles encapsulating pectinase, or carrageenan particles encapsulating carrageenase.

Clause 708. The method of clause 704 or 705, wherein the microsphere comprises alginate particles encapsulating alginate lyase, pectin particles encapsulating pectinase, or carrageenan particles encapsulating carrageenase; and wherein the anion comprises citrate.

Clause 709. The method of clause 704 or 706, wherein the microsphere comprises alginate particles encapsulating alginate lyase, pectin particles encapsulating pectinase, or carrageenan particles encapsulating carrageenase; and wherein the phosphate buffer comprises phosphate buffered saline.

Clause 710. A kit comprising:
a plurality of microspheres of any one of clauses 601-619; and
an enzyme capable of rapidly degrading the microspheres when dissolved to form a solution.

Clause 711. The kit of clause 710, wherein the enzyme is complementary to the biomaterial used to form the plurality of microspheres.

Clause 712. The kit of clause 710 or 711, wherein:
the microspheres comprise alginate particles encapsulating alginate lyase and the enzyme is alginate lyase,
the microspheres comprise pectin particles encapsulating pectinase and the enzyme is pectinase,
the microspheres comprise hyaluronic acid particles encapsulating hyaluronidase and the enzyme is hyaluronidase,
the microspheres comprise gelatin particles encapsulating a matrix metalloproteinase or protease and the enzyme is a matrix metalloproteinase or protease,
the microspheres comprise albumin particles encapsulating peptidase and the enzyme is peptidase,
the microspheres comprise collagen particles encapsulating protease and the enzyme is protease,
the microspheres comprise fibrinogen particles encapsulating plasmin and the enzyme is plasmin,
the microspheres comprise silk fibrin particles encapsulating protease and the enzyme is protease,
the microspheres comprise starch particles encapsulating amylase and the enzyme is amylase,
the microspheres comprise chitosan particles encapsulating chitosanase or lysozyme and the enzyme is chitosanase or lysozyme,
the microspheres comprise agar/agarose particles encapsulating agarase and the enzyme is agarase,
the microspheres comprise carrageenan particles encapsulating carrageenase and the enzyme is carrageenase,
the microspheres comprise pullulan particles encapsulating pullulanase and the enzyme is pullulanase,
the microsphere comprises dextran particles encapsulating dextranase and the enzyme is dextranase,
the microspheres comprise b-glycan particles encapsulating b-glycanase and the enzyme is b-glycanase,
the microspheres comprise cellulose particles encapsulating cellulase and the enzyme is cellulase, or
the microspheres comprise lignin particles encapsulating ligninase and the enzyme is ligninase.

Clause 713. A kit comprising:
a plurality of divalent metal ion crosslinked microspheres of any one of clauses 601-619; and
an inorganic salt capable of rapidly degrading the microspheres when dissolved to form a solution.

Clause 714. The kit of clause 713, wherein the microspheres comprise alginate particles encapsulating alginate lyase, pectin particles encapsulating pectinase, or carrageenan particles encapsulating carrageenase.

Clause 715. The kit of clause 713 or 714, wherein the inorganic salt releases citrate or phosphate when dissolved to form a solution.

### EXAMPLES

### Example 1: Alginate Lyase Enzyme Concertation Dependent Degradation of Alginate Particles

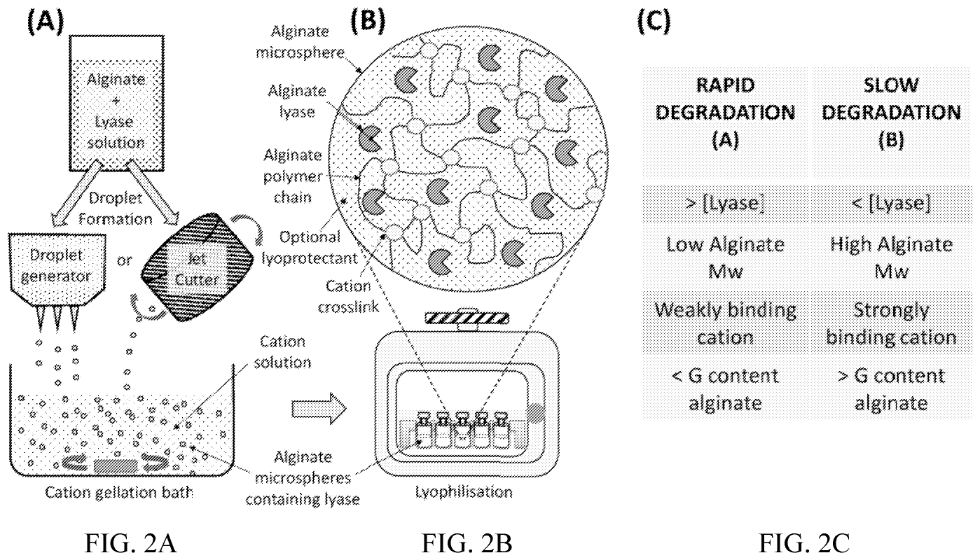

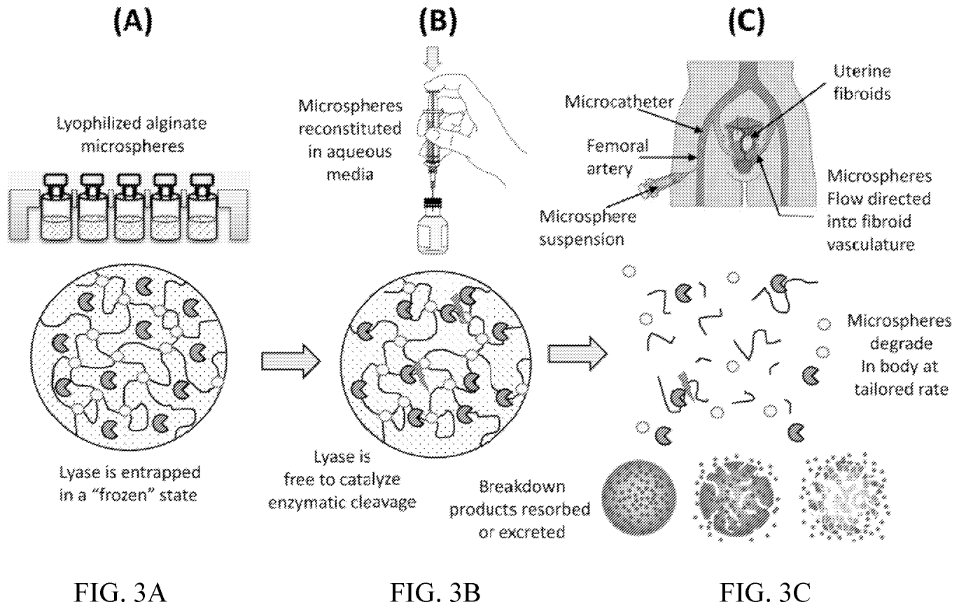

The schematic diagram for the preparation of the alginate particles is shown in FIG. 2A-2C and FIG. 3A-3C. Sodium alginate of viscosity (5-40 cP, condition 1% w/v in water @ 25° C.) was dissolved in de-ionized water to prepare the stock solution of concentration 4% w/v. Likewise, a stock solution of alginate lyase enzyme of concentration 50 U/mL was prepared by dissolving 5 mg of enzyme powder (equivalent 50 U) in 1 mL of DI water. To prepare the alginate lyase-sodium alginate precursor solution having final concentrations of 5 U/mL (0.25 U/mg of alginate) or 0.5 U/ml (0.025 U/mg of alginate) of alginate lyase enzyme and 2% w/v of sodium alginate, 0.1 mL or 0.01 mL of alginate lyase enzyme was mixed with 0.5 ml of 4% w/v of sodium alginate for 30 seconds and make up the volume to 1 mL with deionized water.

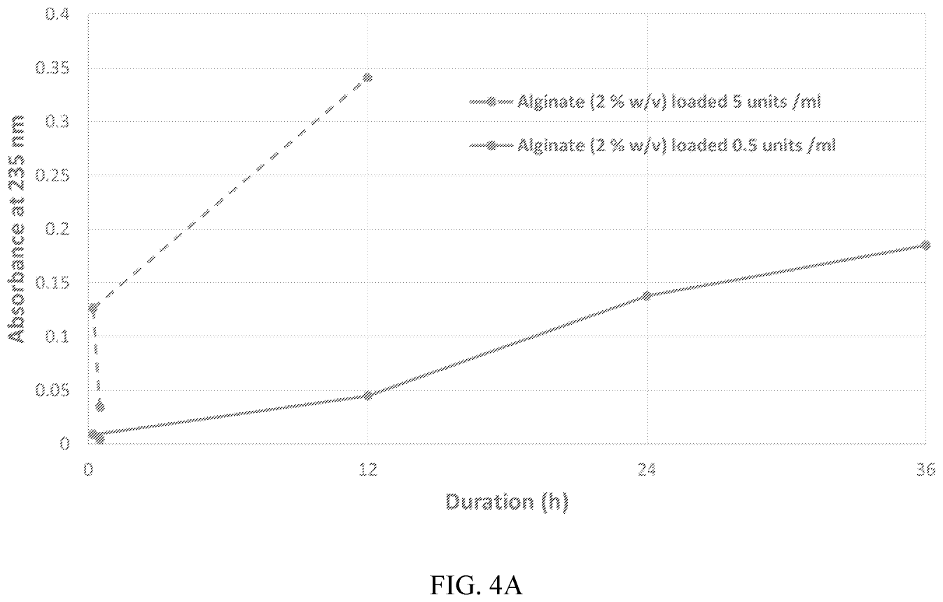

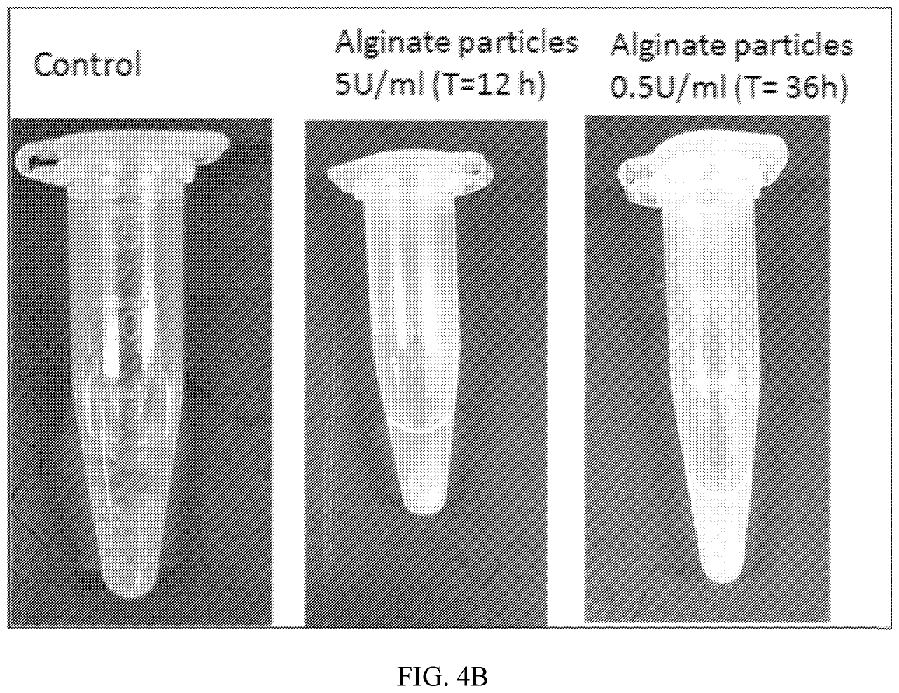

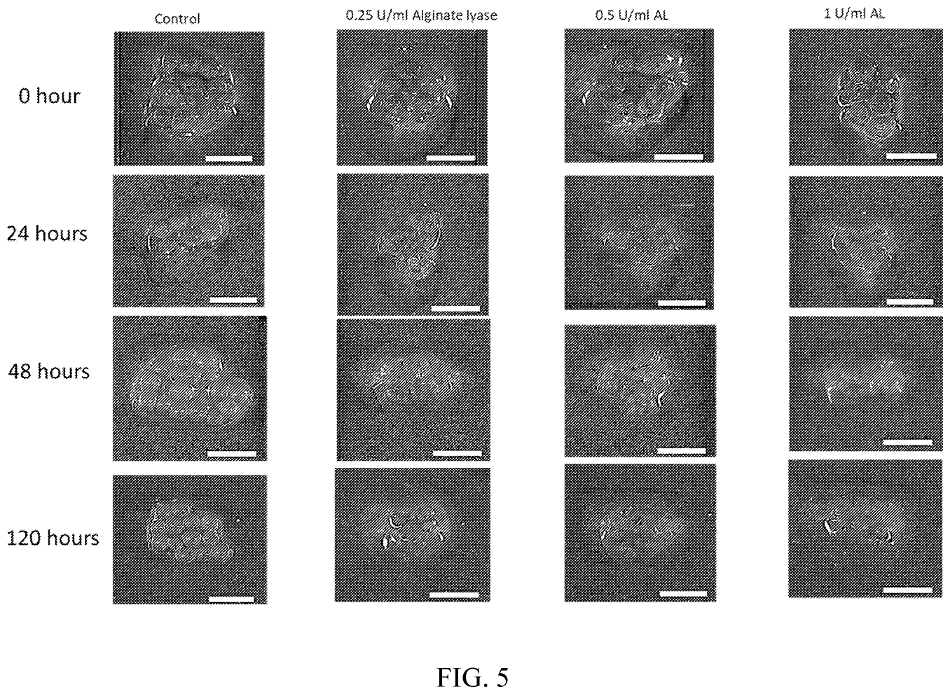

The precursor alginate lyase-alginate solution was added dropwise into the gelling bath containing 10% w/v calcium chloride under constant stirring for 5 minutes to achieve alginate lyase loaded Ca²⁺-crosslinked alginate particles. Then, the particles were isolated by sieving or centrifugation and washed with deionized water three times for 1 minute each to remove excess or calcium chloride. Washed alginate lyase loaded with Ca²⁺-crosslinked alginate particles were dispersed in 10 mM phosphate buffer at pH 6.8 and incubated at 37° C. for the desired duration to evaluate the degradation of alginate particles. The degradation of calcium ion crosslinked alginate particles loaded with 5 units (U) (0.25 U/mg of alginate) and 0.5 U (0.05 U/mg of sodium alginate) of alginate lyase enzyme shown in FIG. 4A. The alginate particles loaded with 5 U of enzyme rapidly degraded in 12 h, whereas the particle loaded with 0.5 U of enzyme degraded at a slower rate and unable to reach the absorbance level similar to 5 U loaded alginate particle after 36 hours. From FIG. 4B, 5 U loaded alginate lyase loaded alginate particles were completely degraded in 12 h, whereas 0.5 U alginate lyase loaded alginate particles samples showed the partially degraded particles in 36 h. In the control sample (without enzyme), the calcium ion complexed alginate particles remained intact. These results demonstrated the alginate lyase enzyme concertation dependent degradation of alginate particles.

### Example 2: Alginate Lyase Enzyme Concentration-Dependent Degradation of Alginate Particles Prepared from High Viscosity Alginate

High viscosity sodium alginate (Viscosity 144 cP, condition 1% w/v in water @ 25° C.) was dissolved in de-ionized water to prepare the stock solution of concentration 3% w/v. Likewise, a stock solution of alginate lyase enzyme of concentration 50 U/ml was prepared by dissolving 5 mg of enzyme powder (equivalent 50 U) in 1 ml of DI water. To prepare the alginate lyase-sodium alginate precursor solution having final concentrations of 1 U/ml, (or 0.05 U/mg of alginate), 0.5 U/ml (or 0.025 U/mg of alginate) and 0.25 U/ml (or 0.0125 U/mg of alginate) of alginate lyase enzyme was mixed with 0.5 ml of 3% w/v of sodium alginate for 30 seconds and make up the volume to 1 ml with deionized water.

The precursor alginate lyase-alginate solution was added dropwise into the gelling bath containing 2% w/v calcium chloride under constant stirring for 5 minutes to achieve alginate lyase loaded Ca²⁺-complexed alginate particles. Then, the particles were isolated by sieving or centrifugation and washed with de-ionized water three times for 1 minute each to remove excess or calcium chloride. Washed alginate lyase loaded Ca²⁺-complexed alginate particles were dispersed in 10 mM phosphate buffer at pH 6.5 and incubated at 37° C. for the desired duration to evaluate the degradation of alginate particles. The degradation of calcium ion complexed alginate particles loaded with 1 U, 0.5 U and 0.25 U of alginate lyase enzyme shown in FIG. 5. The alginate particles loaded with 1 U of enzyme rapidly degraded over 120 hours when compared to the particle loaded with 0.5 U and 0.25 U of enzyme. In the control sample (without enzyme), the Ca²⁺-crosslinked alginate particles remained intact. These results demonstrated the alginate lyase enzyme concertation dependent degradation of alginate particles.

### Example 3: PH-Dependent Regulation of Alginate Lyase Enzyme Conformation/Activity

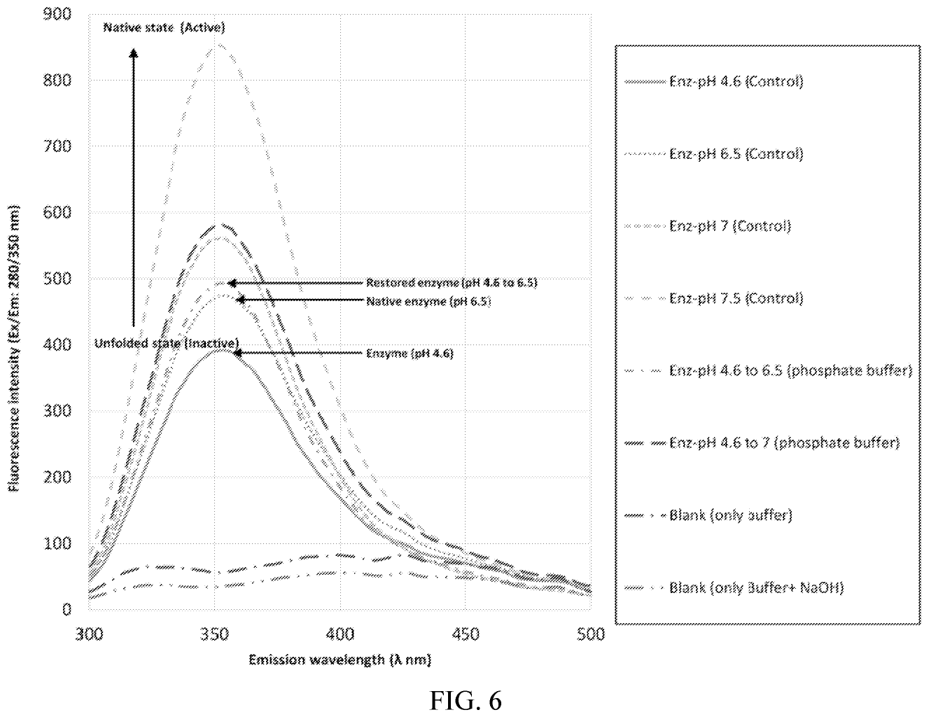

To study the pH-dependent regulation of lyase enzyme conformation/activity, the alginate lyase enzyme was exposed to different pH and subjected to the fluorescence spectroscopy. In general, an open conformation of enzyme inactivates or reduces the enzyme catalytic activity, whereas further stabilization of the native structure improves the catalytic activity of the enzyme. From FIG. 6, the fluorescence of native enzyme (1 U/ml or 0.05 U/mg of alginate) in acidic pH 4.6 (acetate buffer) is at a lower level when compared to the native enzyme at pH 7.0 (10 mM, phosphate buffer). The reduction in fluorescence in acidic pH indicated the open conformation of the enzyme. When the pH of enzyme solution changed to pH 4.6 to 7.0, the fluorescence recovered or enhanced is found to be at a level similar to the native enzyme at pH 7.0. This demonstrated the reversible conformation of the alginate lyase enzyme in response to change in the pH of the solution with reactivation of enzyme activity. Therefore, by changing the pH of alginate lyase-alginate precursor or the gelling bath solutions, the initial degradation of particles during the manufacturing process of alginate lyase loaded divalent metal ion particles. On reconstituting the alginate-lyase enzyme loaded divalent metal ion complexed alginate particles in an aqueous solution having neutral pH (6.5 to 7.5), the activity of the alginate lyase enzyme can be restored to get the tailored degradation of the alginate particles.

### Example 4: Effect of pH on the Preparation of Ca²⁺-Crosslinked Alginate Microspheres Loaded Alginate Lyase Enzyme

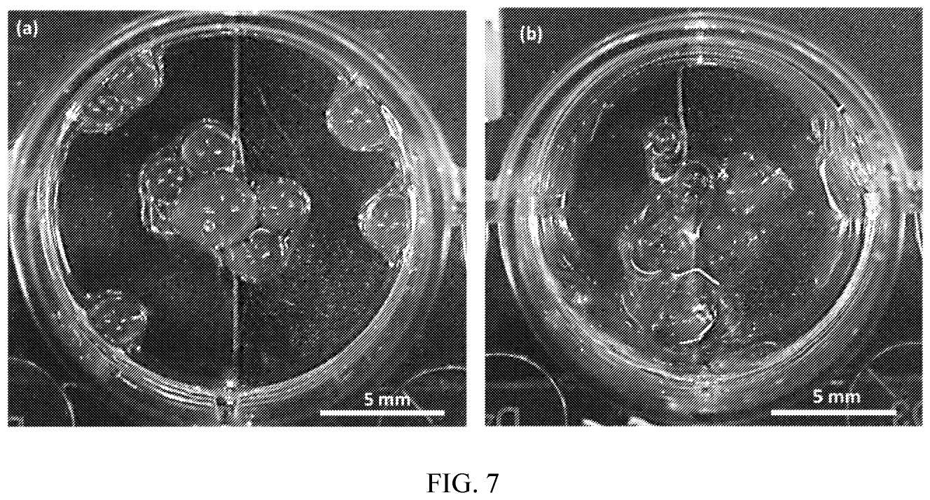

Alginate lyase enzyme and high viscosity alginate (viscosity 144 cP, condition 1% w/v in water @ 25° C.) were dissolved in 0.1 M sodium acetate buffer of pH 4 with final concentrations of 5 U/ml and 1.5% w/v respectively. Similarly, alginate lyase enzyme-high viscosity alginate (viscosity 144 cps, condition 1% w/v in water @ 25° C.) solution was also prepared in 0.01 M phosphate buffer of pH 6.5 with final concentrations of 5 U/ml and 1.5% w/v respectively. Both the solutions were incubated at 4° C. for 15 mins, before dropping into 2% w/v calcium chloride solution to get calcium ion-crosslinked alginate microspheres loaded with alginate lyase enzyme. The crosslinked microspheres were washed three times in deionized water for 1 min each. The microspheres prepared in the acetate buffer showed a spherical shape (FIG. 7 (a)). On the other hand, microspheres obtained from phosphate buffer were irregularly shaped and partially degraded (FIG. 7 (b)). These results indicate that the low pH reduced the catalytic activity of the enzyme and prevented degradation of alginate, thereby aiding in getting the spherical microspheres. This method of making microspheres increases the processing window which may help in upscaling the production of the alginate microspheres loaded with the alginate lyase enzyme.

### Example 5: Degradation of Ca²⁺-Crosslinked Alginate Lyase-Alginate Microspheres Prepared from Alginate-Alginate Lyase Precursor Solution Pretreated with Acidic pH (Acetate Buffer, pH 4)

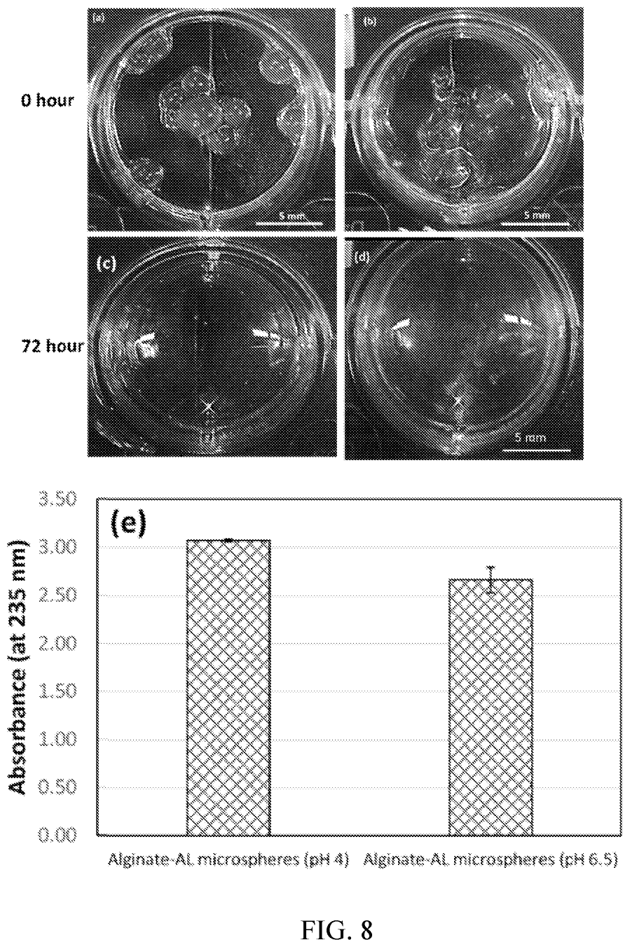

Alginate lyase enzyme and high viscosity alginate (viscosity 144 cP, condition 1% w/v in water @ 25° C.) were dissolved in 0.1 M sodium acetate buffer of pH 4 with final concentrations of 5 U/ml (0.25 U/mg of alginate) and 1.5% w/v respectively. Similarly, alginate lyase enzyme-high viscosity alginate (viscosity 144 cP, condition 1% w/v in water @ 25° C.) solution was also prepared in 0.01 M phosphate buffer of pH 6.5 with final concentrations of 5 U/ml and 1.5% w/v respectively. Both the solutions were incubated at 4° C. for 15 mins, before dropping into 2% w/v calcium chloride solution to get calcium ion-crosslinked alginate microspheres loaded with alginate lyase enzyme. The crosslinked microspheres were washed three times in deionized water for 1 min each. The microspheres prepared in the acetate buffer showed a spherical shape (FIG. 8 (a)). On the other hand, microspheres obtained from phosphate buffer were irregularly shaped and partially degraded (FIG. 8 (b)). These results indicate that the low pH reduced the catalytic activity of the enzyme and prevented degradation of alginate, thereby aiding in getting the spherical microspheres. This method of making microspheres increases the processing window which may help in upscaling the production of the alginate microspheres loaded with the alginate lyase enzyme, by minimizing the degradation of alginate by the alginate lyase in the precursor solution. These two microspheres were incubated in 0.01 M phosphate buffer and/or- supplemented with 0.1 N NaOH to achieve pH 6.5 (in case of precursor solution pre-treated with acetate buffer) for 72 hours. The acetate buffer treated alginate-alginate lyase microspheres was completely degraded with no visible sign of residues as shown in FIG. 8 (c). On the other hand, phosphate buffer treated alginate-alginate lyase microspheres showed some white residues as observed in FIG. 8 (d). The degradation of these particles was also determined using UV-visible spectroscopy, wherein the degraded product of the alginate microspheres was determined by its absorbance at 235 nm. It was observed that acetate buffer treated alginate lyase-alginate Ca²⁺-crosslinked microspheres showed a greater degradation when compared to phosphate buffered-treated equivalent microspheres (FIG. 8 (e)). These results indicate the pH dependent reversible activity of the alginate lyase enzyme, wherein the enzyme is partially and reversibly inhibited or modulated by exposing to low pH, which allow the loading of the desired amount of enzyme in the alginate microsphere without degrading the alginate matrix. The entrapped enzyme in the alginate microsphere is reversibly activated by exposing to optimum pH, resulting in the degradation of the alginate microspheres.

### Example 6: Acidic pH-Dependent Reversible Activity of Alginate Lyase Enzyme

Alginate lyase enzyme and high viscosity alginate (viscosity 144 cP, condition 1% w/v in water @ 25° C.) was dissolved in 0.1 M acetate buffer (pH 4.0) and 0.01 M phosphate buffer (pH 6.5) with the final concentration of 1 U/ml (0.05 U/mg of alginate) and 0.1% w/v respectively. The samples names of the respective reactions are Alginate-AI. A.B and Alginate-AL. P.B. The temperature of these solutions was maintained at 1-4° C. and 37° C. for 30 mins. After the incubation, the reaction was terminated by adding 0.1 N NaOH. Likewise, the alginate lyase enzyme was pre-incubated in acetate buffer for 15 mins, and then mixed with high viscosity alginate dissolved in 0.01 M phosphate buffer and supplemented with 0.1 N NaOH to achieve the optimum pH 6.5 having the final concentration of 0.1 U/ml (0.005 U/mg of alginate) and 1% w/v respectively. The solution was incubated at 1-4° C. and 37° C. for 30 mins. After the incubation, the reaction was terminated by adding 0.1 N NaOH (Sample name is Alginate (P.B)-Al. (A.B)). After terminating the reactions, the enzyme activity of the alginate lyase enzyme was determined by the absorbance of the degraded product at 235 nm wavelength.

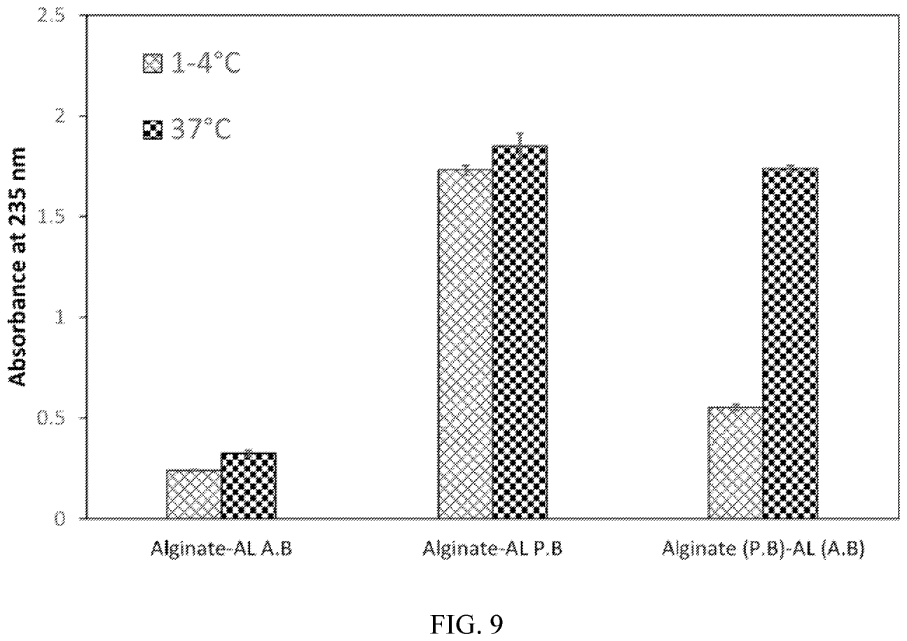

From FIG. 9, it was observed that enzyme activity of Alginate-AL. A.B is reduced at 1-4° C. and 37° C., when compared to Alginate-AI. P.B sample. Furthermore, it is observed from Alginate-AI. P.B sample that there is negligible effect of low temperature on reducing the alginate lyase activity. Furthermore, the alginate lyase enzyme-pre-treated with acetate buffer mixed with alginate dissolved in phosphate buffer (Alginate (P.B)-AL. (A.B)) showed 4 times reduction in the enzyme activity at 4° C. when compared to Alginate-Al. P.B sample. This indicates that acetate buffer partially reduced the activity of the enzyme at 1-4° C. Remarkably, the alginate lyase enzyme activity increases considerably at 37° C. and the levels were found to be similar to Alginate-AI. P.B sample. This increase in the activity showed the pH-dependent reversible alginate lyase activity, wherein the enzyme is partially and reversibly inhibited or modulated by exposing to low pH, which is restored by changing to the optimum pH of the solution. These results are further supported by FIG. 9A-9C.

### Example 7: Lyophilization of Ca²⁺-Crosslinked Alginate Microspheres Loaded with Alginate Lyase Enzyme Using Cryoprotectants PVP 40 kDa (0.5%, W/V) and Trehalose (0.5%, W/V)

Dissolve high viscosity (Viscosity 144 cP, condition 1% w/v in water @ 25° C.) 1.5% w/v sodium alginate, PVP 40 kDa (0.5% w/v), trehalose (0.5% w/v) in deionized water and stir on a magnetic stirrer for half an hour/45 min at 1-4° C. to obtain a homogenous dispersion. Then, added 5 U alginate lyase enzyme into the dispersion and mix for 1 min. This solution added dropwise to 2% w/v CaCl₂) containing PVP 40 kDa (0.5% w/v) and trehalose (0.5% w/v), and stir for 15 minutes to get PVP 40 kDa and trehalose containing Ca²⁺-crosslinked alginate microspheres loaded with alginate lyase enzyme. These microspheres were further washed with deionized water 3 times for 1 min. each and exposed to liquid nitrogen for 30 sec to 2 minutes. The frozen microspheres were lyophilized for 24 hours using a lyophilizer which was set at −57° C. under ultra-high vacuum. From FIGS. 11A and 11A′, the freeze dried or lyophilized microspheres showed no change in the shape when compared to non-lyophilized microspheres, thus indicating preservation of shape of the microspheres.

### Example 8: Lyophilization of Ca²⁺-Crosslinked Alginate Microspheres Loaded with Alginate Lyase Enzyme Using Cryoprotectants Hydroxypropyl-β-Cyclodextrin (0.5%, W/V)

High viscosity (144 cP, condition 1% w/v in water @ 25° C.) 1.5% w/v sodium alginate and Hydroxypropyl-β-cyclodextrin (0.5%, W/V) were dissolved in deionized water and stirred on a magnetic stirrer for half an hour/45 min at 1-4° C. to obtain a homogenous dispersion. Then, 5 U alginate lyase enzyme (0.25 U/mg of alginate) was added into the dispersion and mixed for 1 min. This solution was added dropwise to 2% w/v CaCl₂) containing 0.5% w/v of Hydroxypropyl-β-cyclodextrin and stirred for 15 minutes to get Hydroxypropyl-β-cyclodextrin containing Ca²⁺-crosslinked alginate microspheres loaded with alginate lyase enzyme. These microspheres were further washed with deionized water 3 times for 1 min. each and exposed to liquid nitrogen for 30 sec to 2 minutes. The frozen microspheres were lyophilized for 24 hours using a lyophilizer which was set at −57° C. under ultra-high vacuum. From FIG. 11(B) and 11(B′), the freeze dried or lyophilized microspheres showed no change in the shape when compared to non-lyophilized microspheres, thus indicating preservation of shape of the microspheres.

### Example 9: Degradation of Lyophilized Ca²⁺-Crosslinked Alginate Microspheres Loaded with Alginate Lyase Enzyme Containing PVP 40 kDa (0.5%, W/V) and Trehalose (0.5%, W/V) Cryoprotectants

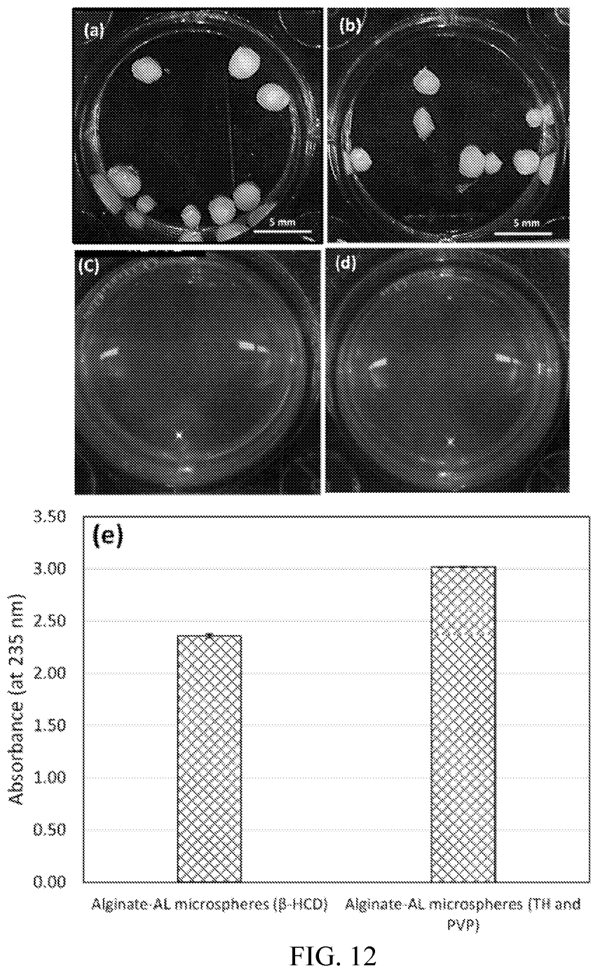

High viscosity (Viscosity 144 cP, condition 1% w/v in water @ 25° C.) 1.5% w/v sodium alginate, PVP 40 kDa (0.5% w/v), and trehalose (0.5% w/v) were dissolved in deionized water and stirred on a magnetic stirrer for half an hour/45 min at 1-4° C. to obtain a homogenous dispersion. Then, 5 U alginate lyase enzyme (0.25 U/mg of alginate) was added into the dispersion and mixed for 1 min. This solution was added dropwise to 2% w/v CaCl₂ containing PVP 40 kDa (0.5% w/v) and trehalose (0.5% w/v), and stirred for 15 minutes to get PVP 40 kDa and trehalose containing Ca²⁺-crosslinked alginate microspheres loaded with alginate lyase enzyme. These microspheres were further washed with deionized water 3 times for 1 min. each and exposed to liquid nitrogen for 30 sec to 2 minutes. The frozen microspheres were lyophilized for 24 hours using a lyophilizer which was set at −57° C. under ultra-high vacuum. FIG. 12 (a) shows the freeze dried or lyophilized microspheres. The lyophilized microspheres were suspended in 0.01 M phosphate buffer (pH 6.5) and incubated at 37° C. for 72 hours. It was observed that the suspended microsphere was degraded, and the turbidity is observed as shown in FIG. 12 (c). FIG. 12 (e) shows the absorbance spectra of degraded product of lyophilized self-degradable alginate microspheres.

### Example 10: Degradation of Lyophilized Ca²⁺-Crosslinked Alginate Microspheres Loaded with Alginate Lyase Enzyme Containing Hydroxypropyl-β-Cyclodextrin (0.5%, W/V) Cryoprotectant

High viscosity (144 cP, condition 1% w/v in water @ 25° C.) 1.5% w/v sodium alginate and Hydroxypropyl-β-cyclodextrin (0.5% w/v) were dissolved in deionized water and stirred on a magnetic stirrer for half an hour/45 min at 1-4° C. to obtain a homogenous dispersion. Then, 5 U alginate lyase enzyme (0.25 U/mg of alginate) was added into the dispersion and mixed for 1 min. This solution was added dropwise to 2% w/v CaCl₂) containing 0.5% w/v of Hydroxypropyl-β-cyclodextrin and stirred for 15 minutes to get Hydroxypropyl-β-cyclodextrin containing Ca²⁺-crosslinked alginate microspheres loaded with alginate lyase enzyme. These microspheres were further washed with deionized water 3 times for 1 min. each and exposed to liquid nitrogen for 30 sec to 2 minutes. The frozen microspheres were lyophilized for 24 hours using a lyophilizer which was set at −57° C. under ultra-high vacuum. FIG. 12 (b) shows the freeze dried or lyophilized microspheres. The lyophilized microspheres were suspended in 0.01 M phosphate buffer (pH 6.5) and incubated at 37° C. for 72 hours. It was observed that the suspended microsphere was degraded, and the turbidity is observed as shown in FIG. 12 (d). FIG. 12. (e) shows the absorbance spectra of degraded product of lyophilized self-degradable alginate microspheres.

### Example 11: Ex Vivo Degradation of Alginate Lyase Loaded Divalent Metal Ion-Complexed Alginate Particles

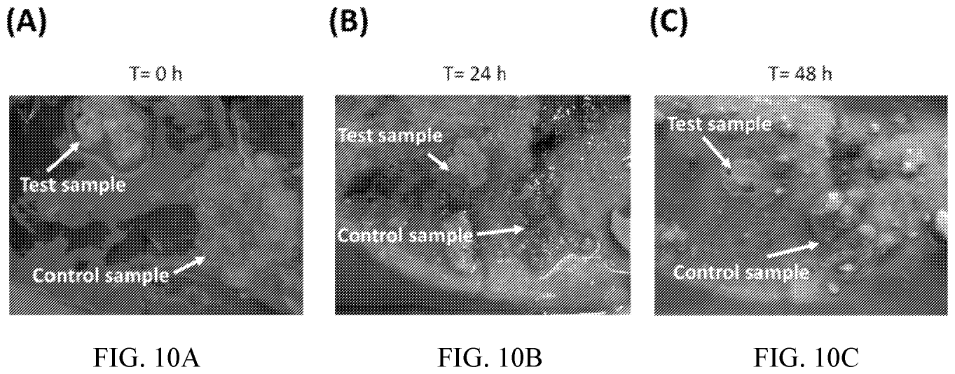

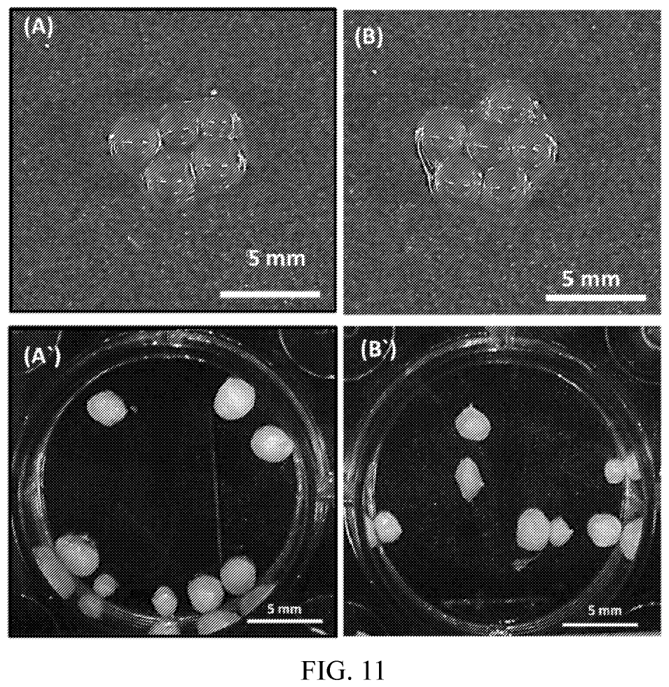

In this test, 5 U of alginate lyase enzyme (0.25 U/mg of alginate) was loaded into the calcium ion-complexed alginate particles and control particles (without enzyme) and placed it onto the liver (bovine) immersed in saline. To evaluate the degradation of particles, the liver was kept in an oven with a temperature set at 37±1° C. and morphological change in the particles was observed for 48 hours. From FIG. 10, the alginate lyase loaded alginate particles lost the shape in 48 hours and a film of white residue can be observed. On the other hand, the control particles maintain the shape for 48 hours. A black film on the control particles can be observed which might be the formation of biofilm.

### Example 12: In Vitro Biocompatibility of Alginate Particles

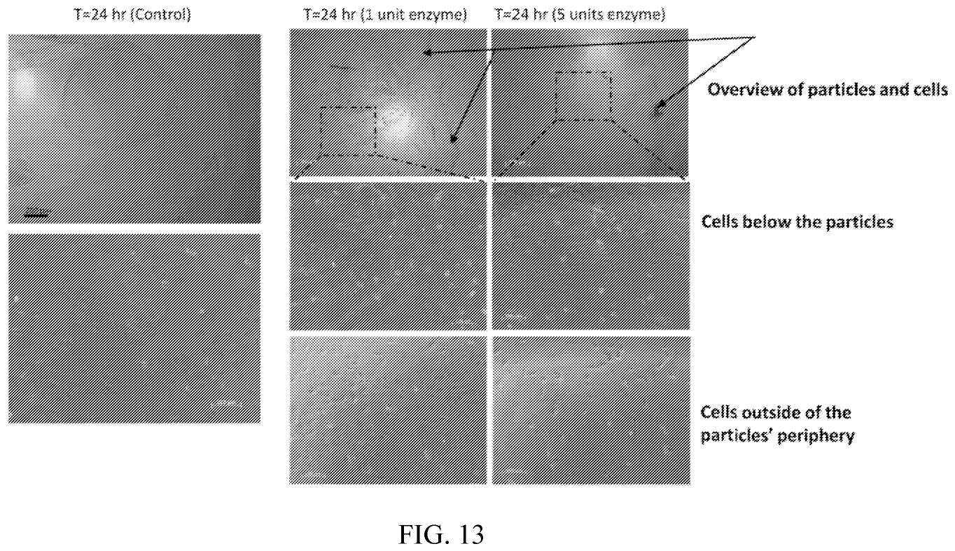

Two different calcium ion-complexed alginate particles were prepared loaded with 1 U (0.05 U/mg of alginate) and 5 U (0.25 U/mg of alginate) of alginate lyase enzyme. To evaluate the biocompatibility of the particles, the morphology and viability of the cells were observed through a light microscope as shown in FIG. 13. Cells were seeded in a 24 well-plate with the cell density of 10+ cells per ml. Cells were cultured under 37° C., 5% CO₂, and 95% relative humidity in alpha-MEM containing 10% fetal bovine serum and 1% penicillin and streptomycin. At least 10 particles of size 2-3 mm were added in the 24 well-plate and incubated for 24 hr. In control samples, intact particles were observed with no detrimental influence on the viability and morphology of osteoblast cells. Alginate particles loaded with 5 U of alginate lyase enzyme were completely degraded (indicated by the debris of the degraded alginate particles), whereas 1 U of alginate lyase enzyme loaded alginate particles were irregularly shaped. The cells are viable with flattened morphology below the degraded particles. This data demonstrated the in vitro biocompatibility of the alginate lyase loaded calcium-complexed alginate particles.

### Example 13: Degradation of Lyophilized Ca²⁺-Crosslinked Alginate Lyase-Alginate Microspheres in from Alginate-Alginate Lyase Precursor Solution Pretreated with Alkaline pH (Carbonate Buffer, pH 10)

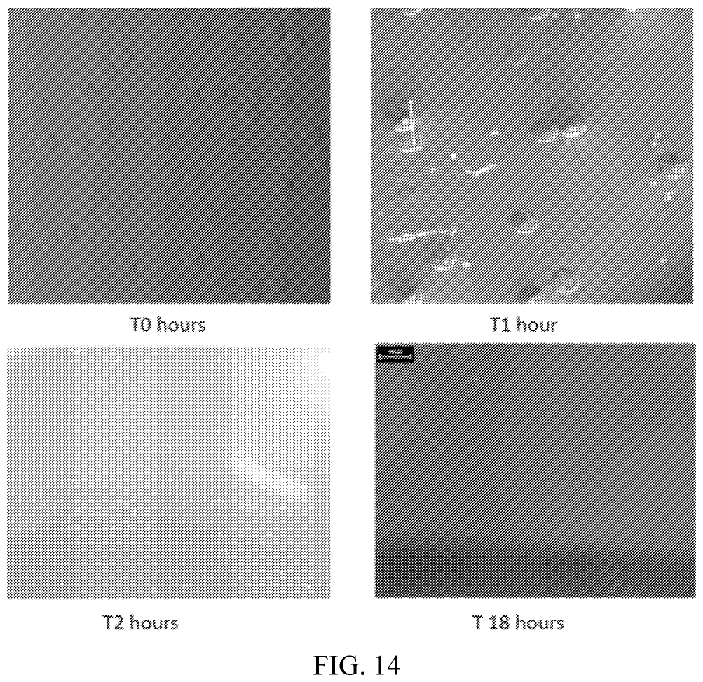

4% w/v Pronova alginate (G/M<1, molecular weight approx. 75 kDa-200 kDa) was dissolved in carbonate buffer (pH 10 and 0.1 M) and stirred on a magnetic stirrer for half an hour/45 min at 1-4° C. to obtain a homogenous dispersion. Then, 100 mM of ethylenediaminetetraacetic acid calcium disodium salt hydrate and alginate lyase were added to obtain the precursor solution containing final concentration of 2% w/v alginate, 0.05 U/ml (0.0025 U/mg of alginate) of alginate lyase, and 50 mM of ethylenediaminetetraacetic acid calcium disodium salt hydrate. Through the microfluidics platform, the droplets of precursor solution of size approx. 200 μm were generated using oil-in-water emulsion technique and exposed to an acidic solution of 2% w/v acetic acid, oil+0.05% surfactant to crosslink the beads, wherein Ca²⁺ ion releases by the ionization of Ca-EDTA and binds to egg-box alginate droplets to generate Ca²⁺-crosslinked alginate beads loaded with alginate lyase enzyme. To isolate the crosslinked alginate beads of size approx. 200 μm, the droplets were treated with a droplet breaking solution and further washed with deionized water to remove the oil and surfactant. These beads were further crosslinked in 2% w/v CaCl₂ solution for 5 mins, thereafter, again washed with deionized water to remove the residual calcium chloride solution. Furthermore, the beads were stored in acetate buffer (0.1 M, pH 4.0) containing trehalose and β-hydroxycyclodextrin for at least 6 hours before being subjected to lyophilization. After lyophilization, the beads were rehydrated or reconstituted in a saline solution and the degradation was evaluated for 18 hours in static solution condition. From FIG. 14, T1 hours shows some minor breakdown, shape change with loss of uniformity, and small particle agglomeration (<10 μm) appearing in the solution. T2 hours follows this trend with increased surface and shape damage, agglomeration of small particles in solution has increased. T18 hours showed a completely bead free solution with no agglomeration observed.

### Example 14: Degradation of Post Lyophilized Resorbable Alginate Beads Reconstituted in Saline Solution in Elastrat Liver Model

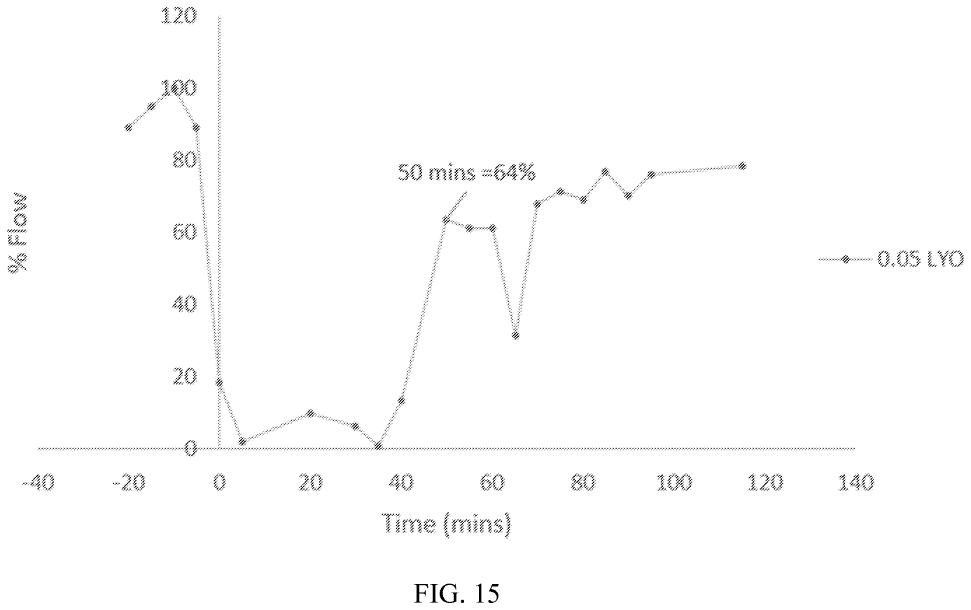

Herein, the lyophilized resorbable alginate beads containing 0.05 U (0.0025 U/mg of alginate) of alginate lyase were reconstituted in saline solution and injected in the Elastrat Liver Model to see the occlusion efficiency of these beads and degradation of beads over time by quantifying the flow restoration of saline. FIG. 15 depicts that the flow rate at 0 min (T=0) was reduced by >90% for 38 mins after injections of beads in the liver model channel. By 50 mins, 64% flow was restored, and, after about 2 hours, the flow rate was restored by 80%. These results indicate a greater degradation rate was observed in a dynamic flow condition when compared to static conditions as given in Example 13.

### Example 15: Evaluation of Ca²⁺ Ion Crosslinking on the Degradation Behavior of Resorbable Alginate Beads in Elastrat Liver Model

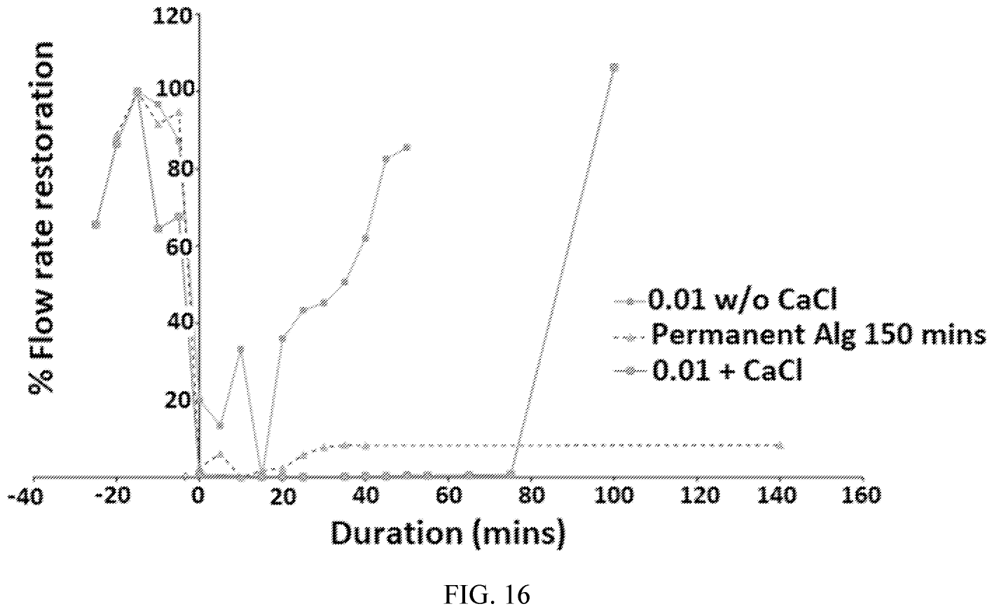

The effect of calcium ion crosslinking on the degradation rate of resorbable alginate is shown in FIG. 16. Resorbable alginate beads Containing 0.01 U of alginate lyase (0.0005 U/mg of alginate) that were crosslinked by Ca²⁺ ion released by the ionization of Ca-EDTA under acidic conditions (0.01 without (w/o) CaCl₂)) showed an inefficient reduction of the flow rate of saline after injection into the liver model, indicating the presence of deformed alginate beads due to the alginate lyase degrading activity. By 40 mins, ˜80% flow rate was achieved, thereby indicating the rapid degradation of resorbable alginate beads. To reduce the activity of the enzyme, these beads were further crosslinked in 2% w/v CaCl₂) solution for 5 mins. These particles were injected into the liver model and a complete reduction in the flow rate was observed until 70 mins, thereafter an accelerated restoration of flow was achieved by 100 mins. On the other hand, permanent beads (only crosslinked with Ca-ETDA) showed a continuous reduction in the flow rate indicating no degradation or disintegration of alginate beads observed over a period of 140 min. These results demonstrated that Ca²⁺ crosslinking is dependent on alginate lyase activity, thus controlling the degradation of alginate beads.

### Example 16: Alkaline pH Dependent Reversible Activity of Alginate Lyase

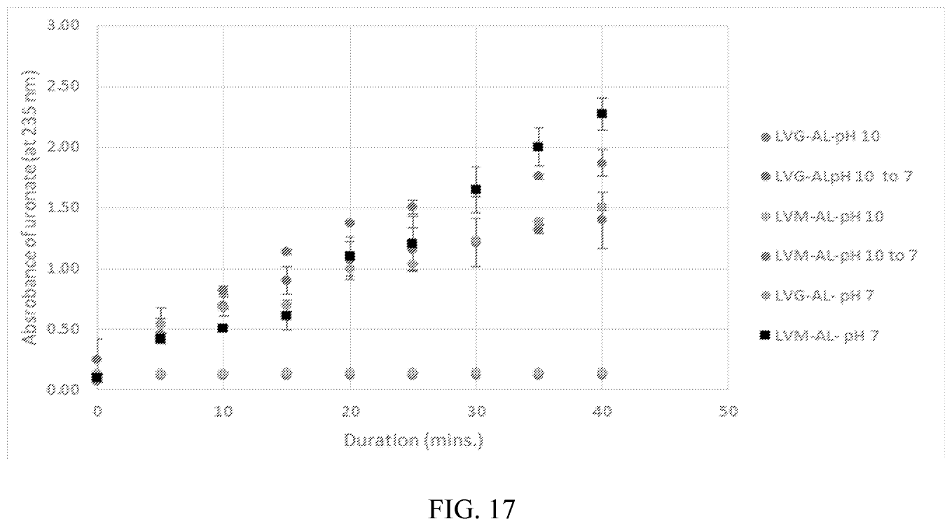

Alginate lyase enzyme and LVG alginate (G/M>1, molecular weight approx. 75 kDa-200 kDa) or LVM alginate (G/M<1, molecular weight approx. 75 kDa-200 kDa) were dissolved in 0.1 M carbonate buffer (pH 10.0) and 0.01 M phosphate buffer (pH 6.5) with the final concentration of 0.1 U/ml (0.005 U/mg of alginate) and 0.1% w/v respectively. The sample names of the respective reactions are LVM- or-LVG-AL pH 10 and LVG-Alginate-AL pH 7. Likewise, the alginate lyase enzyme was pre-incubated in pH 10.0, 0.1 M carbonate buffer for 15 mins, and then mixed with LVG alginate (G/M>1, molecular weight approx. 75 kDa-200 kDa) or LVM alginate (G/M<1, molecular weight approx. 75 kDa-200 kDa) in 0.01 M phosphate buffer to achieve the optimum pH 6.5 having the final concentration of 0.1 U/ml and 0.1% w/v respectively. The sample names of the respective reactions are LVM- or-LVG-AL pH 10 to 7. All the above-mentioned samples were incubated and the enzyme activity of the alginate lyase enzyme was determined by the absorbance of the degraded product at 235 nm wavelength (FIG. 17).

### Example 17: Effect of Sterilization on Alginate Beads Loaded with 0.05 U of Alginate Lyase Enzyme

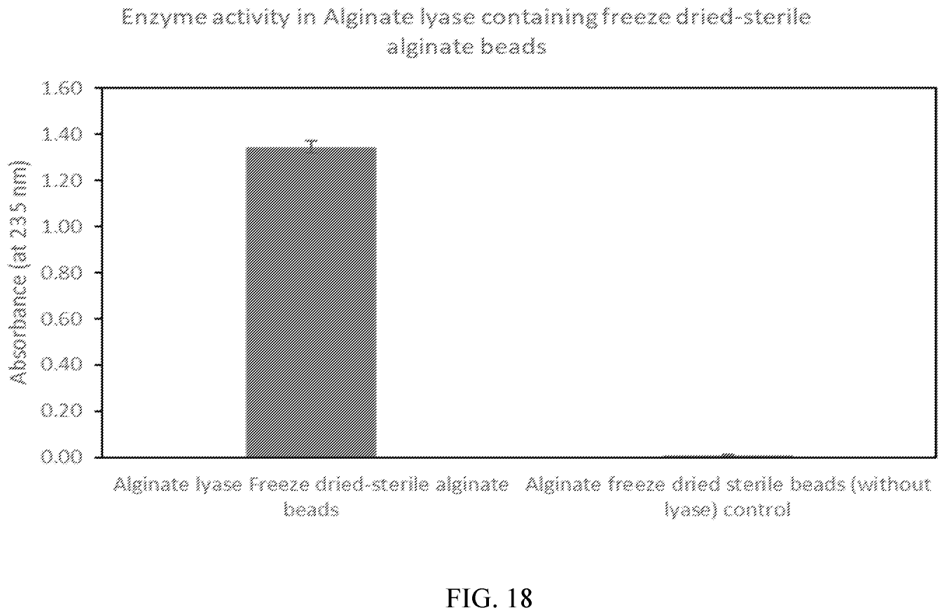

The lyophilized alginate beads containing 0.05 U (0.0025 U/mg of alginate) of alginate were sterilized using 10 kGy e-beam and subjected to self-degradation in saline solution at physiological conditions (pH 7 and temperature 37° C.) for 24 hours. FIG. 18 shows the presence of alginate lyase activity as indicated by an increase in absorbance recorded at 235 nm when compared to freeze dried sterile alginate beads control (without lyase). These results demonstrated that resorbable beads can be freeze dried, sterilized, and degraded on reconstitution in an aqueous solution (here, saline) under physiological conditions wherein the sterilization does not affect the alginate lyase enzyme activity.

### Example 18: Preparation and Degradation of Pectin Lyase Encapsulated Calcium-Crosslinked Pectin Beads

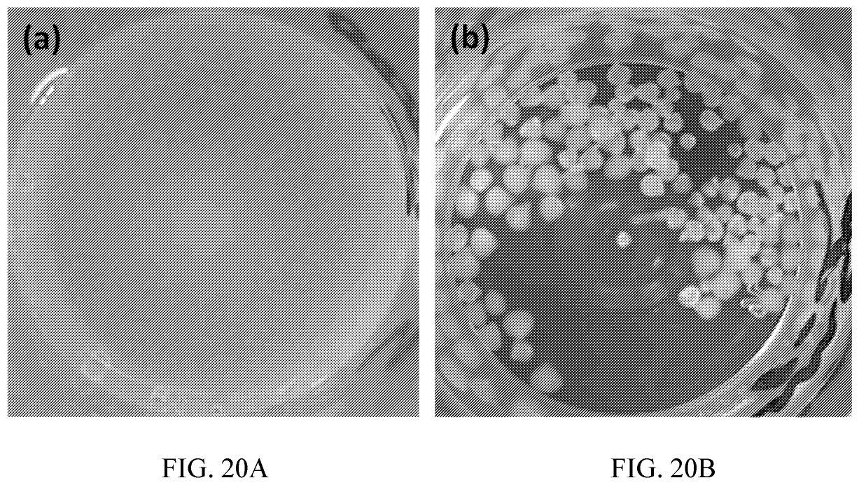

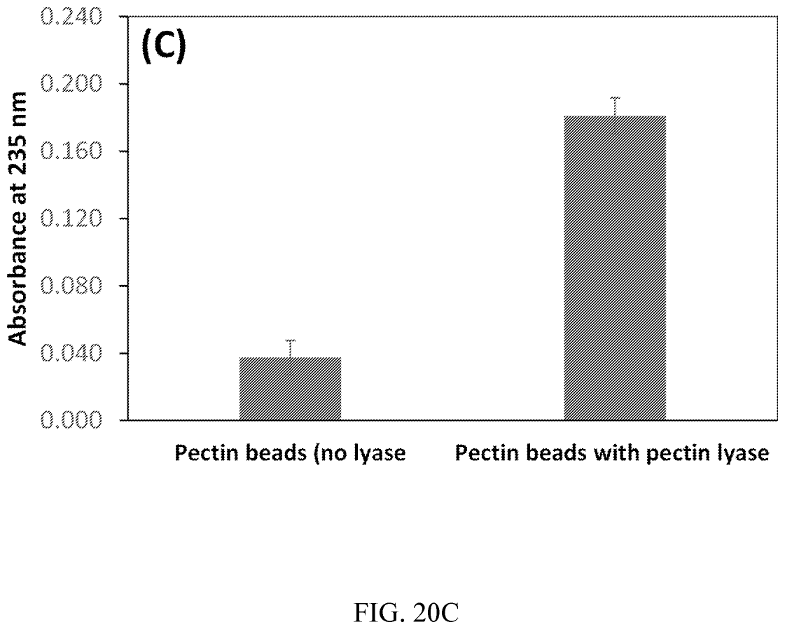

To prepare resorbable pectin lyase-loaded calcium-crosslinked pectin beads (isolated from citrus peel), 5 U of pectin lyase (pectinase) was mixed with 4% w/v pectin in 0.1 M HEPES buffer of pH 7.4. The temperature of this solution was maintained at 1-4° C. Then, the solution was added dropwise to 2% w/v CaCl₂) (gelling bath) to get the resorbable beads. The beads were allowed to crosslink in the gelling bath for 30 mins. After cross-linking, the beads were washed with 0.1 M HEPES buffer with a pH of 4.0 to remove the calcium chloride residues. Using the same approach, control non-degradable calcium-crosslinked pectin beads were prepared without lyase. Furthermore, the degradation of both beads was evaluated. To test these beads, an equal amount of 500 mg of wet beads was dispersed in 10 mL of pH 7.4 0.1 M HEPES buffer at 37° C. for 24 hours. The degradation of beads was recorded by imaging and UV-visible spectroscopy. It can be observed in FIGS. 20A-20C that the resorbable beads were completely disintegrated and formed a turbid solution, whereas pectin beads without lyase were intact after 24 hours. Likewise, the resorbable pectin lyase encapsulated calcium crosslinked pectin beads showed considerably higher absorbance as compared to the non-degradable beads.

### Example 19: Alginate Lyase Enzyme Concentration-Dependent Degradation of Alginate Particles

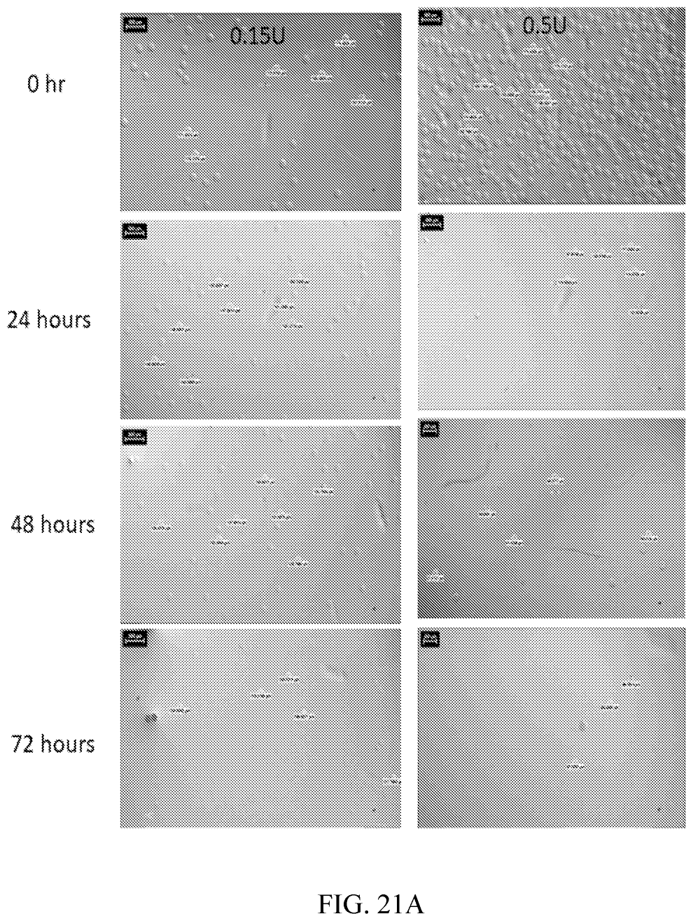

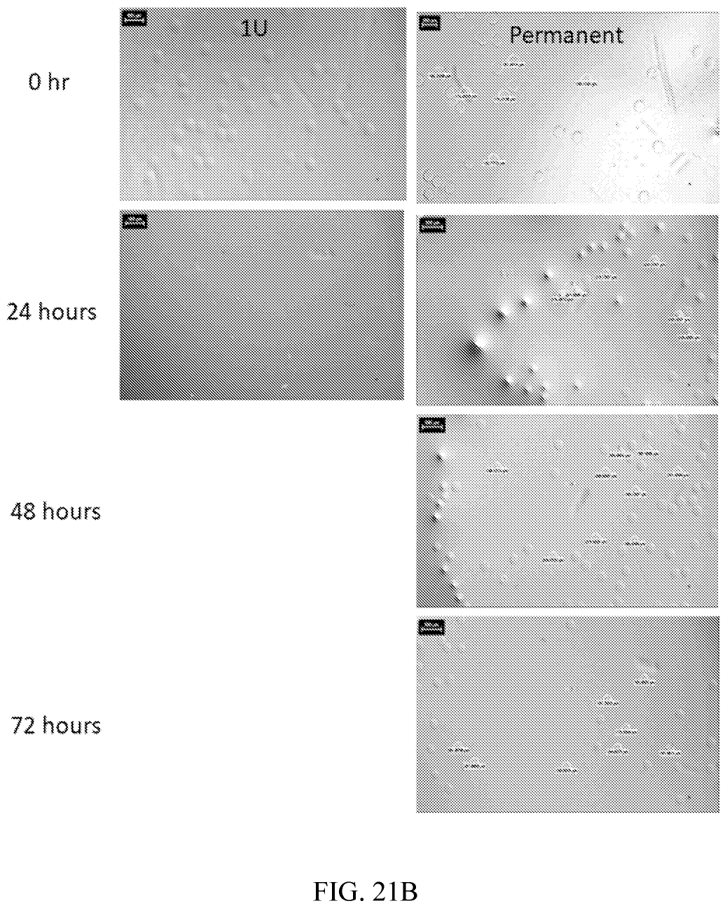

A pH 3.6 to 4.0 solution was made containing 2% w/v UP LVM sodium alginate, 4% w/v Trehalose (excipient), and 4% w/v dextran 70 (excipient). Then, the desired units (0.15 U, 0.5 U, and 1 U of alginate lyase were added and mixed for 3-5 mins to get the precursor solution. Microspheres were made using an electro-encapsulation process, wherein the droplets of the precursor solution were made by passing the precursor solution through the needle (ID 0.09 mm) at a fixed flow rate (3 mL/hr) under the influence of an electrostatic potential (3 kV). The droplets were crosslinked in a gelling bath containing 2% w/v calcium chloride and 4% w/v Trehalose (excipient). The beads were crosslinked for 60 mins. 100 μL of beads was dispersed in 1 mL of 0.1 M, pH 6.8 HEPES solution and the degradation of beads over 72 hours was evaluated microscopically. The alginate particles loaded with 1 U of enzyme rapidly degraded in 24 hours compared to those loaded with 0.15 U and 0.5 U of enzyme, which degraded in 72 hours (FIGS. 21A-21B). In the control/permanent beads sample (without enzyme), the Ca²⁺-crosslinked alginate particles remained intact for over 72 hours. These results demonstrated the alginate lyase enzyme concentration-dependent degradation of alginate particles.

### Example 20: Bail Out Procedure to Rapidly Degrade an Alginate Microsphere (with No Encapsulated Alginate Lyase)

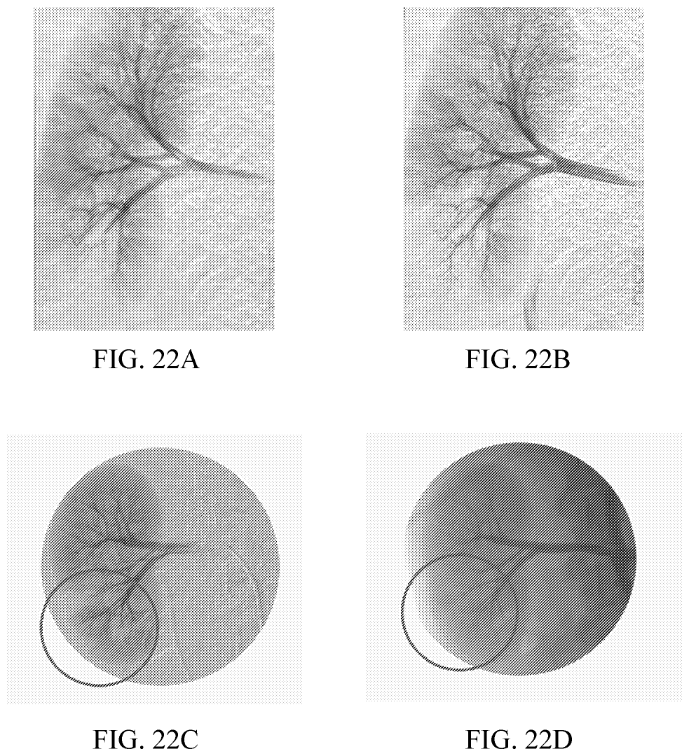

A vial of lyophilized alginate microsphere (without encapsulated alginate lyase) was reconstituted in saline and mixed with contrast. The alginate microspheres were injected into a kidney to create an occlusion. FIG. 22A shows a kidney segment immediately after injection of 1.8 mL of the alginate microspheres. After 2 hours of occlusion, alginate lyase dissolved in phosphate buffered saline (PBS) was injected at the occlusion site to clear the embolization. FIG. 22B shows partial revascularization of flow within 10 minutes after the injection of the alginate lyase bail out solution. The reperfusion of the blood vessel after injection of the bail out solution can be observed in FIG. 22C. 24 hours after alginate lyase injection, complete reperfusion of the occluded site can be observed as shown in FIG. 22D. This study demonstrated that the calcium ion crosslinked alginate microspheres devoid of encapsulated alginate lyase or permanent microspheres can be degraded by injecting alginate lyase enzyme at the embolic site under in vivo conditions.

### Example 21: Angiography of Porcine Kidney Embolized with 0.15 U and 0.0875 U Alginate Lyase Loaded Ca²⁺ Crosslinked Alginate Microspheres

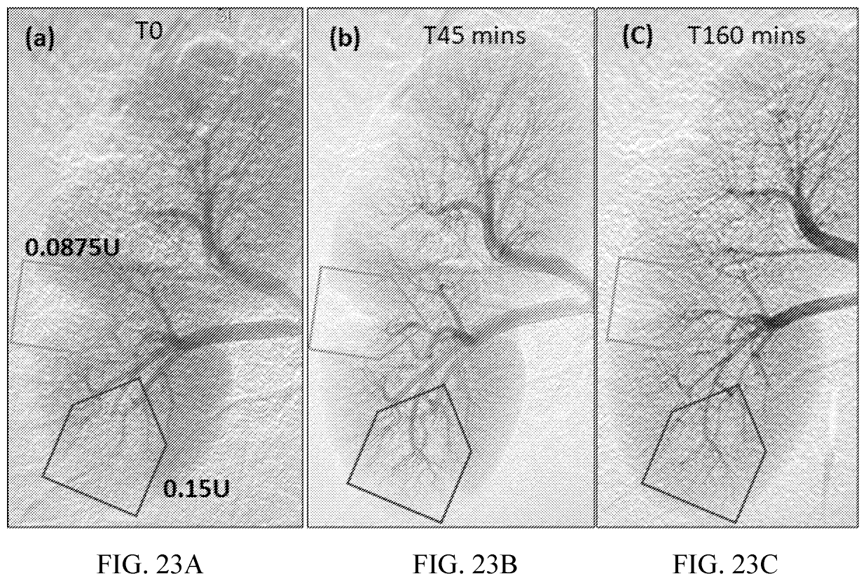

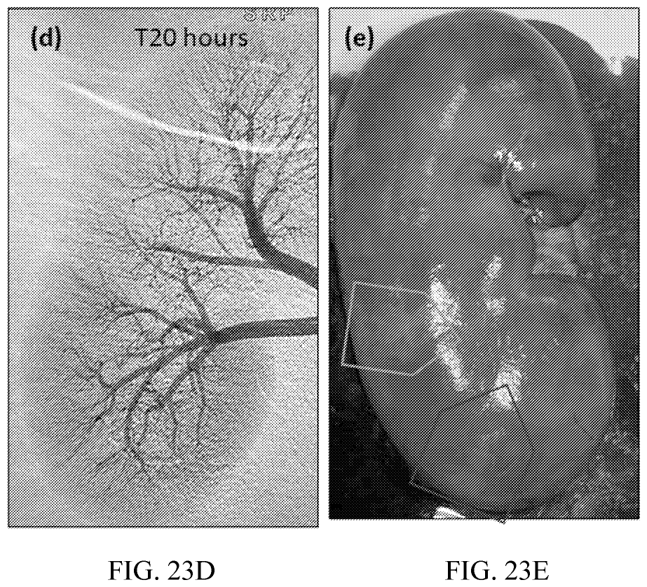

Vials of lyophilized resorbable alginate microspheres loaded with 0.15 U alginate lyase or 0.0875 U alginate lyase were reconstituted in saline and mixed with the contrast. The 0.15 U and 0.0875 U alginate lyase loaded alginate microspheres were injected into the caudal medial site of a porcine kidney to create an occlusion (FIG. 23A). It was observed that the blood vessels occluded with 0.15 U alginate lyase loaded alginate microspheres reperfused at an earlier time point (30 mins) compared to 0.0875 U alginate lyase loaded alginate microspheres (FIG. 23B). At 20 hours, a complete reperfusion of the occluded sites was observed (FIG. 23D) indicating the complete degradation of the 0.0875 U alginate lyase loaded alginate microspheres. The effect of embolization time can be observed from the discoloration of kidney parenchyma (FIG. 23E). The discoloration can be observed in the case of 0.0875 U alginate lyase loaded alginate microspheres, whereas the 0.15 U alginate lyase loaded alginate microspheres did not show any discoloration of the kidney parenchyma. This study shows the enzyme-dependent degradation of alginate microspheres under in vivo conditions and its application in creating transient embolization.

What is claimed is:

1. A microsphere capable of self-degradation upon rehydration, comprising:
an enzyme pre-treated by varying temperature, by varying pH, and/or with a metal-ion enzyme inhibitor; and
a crosslinked biomaterial;
wherein:
the crosslinked biomaterial forms a biologically derived microsphere encapsulating the enzyme; and
the microsphere is substantially free of water and/or sterilized.

2. The microsphere of claim 1, wherein at least one of (i)-(iv) applies:
(i) the enzyme is an enzyme that acts on the biomaterial, degrading the biomaterial;
(ii) the biomaterial comprises a polysaccharide, a protein, or a glycoprotein;
(iii) the microsphere further comprises a photoinitiator and the biomaterial comprises a photo-crosslinkable moiety which is photo-crosslinked; or
(iv) the biomaterial is crosslinked by a divalent metal ion.

3. The microsphere of claim 1 or 2, wherein the self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial.

4. The microsphere of any one of claims 1-3, wherein:
the enzyme activity is between about 0.0075 U/mg to 0.25 U/mg of the biomaterial and the microsphere degrades over a period of greater than about 20 minutes to less than about 4 hours;
the enzyme activity is between about 0.005 U/mg to about ≥0.0025 U/mg of the biomaterial and the microsphere degrades over a period of greater than about 5 days to less than about 30 days; or
the enzyme activity is less than about 0.0025 U/mg of the biomaterial, and the microsphere degrades over a period of greater than about 30 days.

5. The microsphere of any one of claims 1-4, wherein at least one of (i)-(iv) applies:
(i) the residual water content of the microsphere is between about 1% by mass and about 10% by mass;
(ii) the microsphere is lyophilized or dehydrated using super critical CO₂;
(iii) the microsphere is sterilized with about 6-10 kGy of gamma radiation; or
(iv) the microsphere further comprises an anti-inflammatory agent, a chemotherapeutic agent, an antioxidant, a corticosteroid, or a combination thereof.

6. The microsphere of any one of claims 1-5, wherein: the biomaterial comprises alginate and the enzyme is alginate lyase, the biomaterial comprises pectin and the enzyme is pectinase, the biomaterial comprises hyaluronic acid and the enzyme is hyaluronidase, the biomaterial comprises gelatin and the enzyme is a matrix metalloproteinase or protease, the biomaterial comprises albumin and the enzyme is peptidase, the biomaterial comprises collagen and the enzyme is protease, the biomaterial comprises fibrinogen and the enzyme is plasmin, the biomaterial comprises silk fibrin and the enzyme is protease, the biomaterial comprises starch and the enzyme is amylase, the biomaterial comprises chitosan and the enzyme is chitosanase or lysozyme, the biomaterial comprises agar/agarose and the enzyme is agarase, the biomaterial comprises carrageenan and the enzyme is carrageenase, the biomaterial comprises pullulan and the enzyme is pullulanase, the biomaterial comprises dextran and the enzyme is dextranase, the biomaterial comprises b-glycan and the enzyme is b-glycanase, the biomaterial comprises cellulose and the enzyme is cellulase, or the biomaterial comprises lignin and the enzyme is ligninase.

7. A method of preparing a microsphere capable of self-degradation upon rehydration, the method comprising:
forming droplets from a precursor solution, the precursor solution comprising:
(i) an enzyme pre-treated by varying temperature, by varying pH, and/or with a metal-ion enzyme inhibitor; and
(ii) a biomaterial;
contacting the droplets with a gelling bath comprising a cryoprotectant and a divalent metal ion, thereby cross-linking the biomaterial to form a biologically derived microsphere encapsulating the enzyme; and
dehydrating, and optionally sterilizing, the microsphere thereby substantially removing water from the microsphere.

8. The method of claim 7, wherein at least one of (i)-(vi) applies:
(i) the precursor solution further comprises one or more cryoprotectants;
(ii) the enzyme is an enzyme that acts on the biomaterial, degrading the biomaterial;
(iii) the biomaterial comprises a polysaccharide, a protein, or a glycoprotein;
(iv) the microsphere comprises: alginate particles encapsulating alginate lyase, pectin particles encapsulating pectinase, gelatin particles encapsulating a matrix metalloproteinase or protease, or carrageenan particles encapsulating carrageenase;
(v) the microsphere self-degrades in less than about 4 hours, over a period of greater than about 2 days to less than about 5 days, over a period of greater than about 5 days to less than about 30 days, or greater than about 30 days; or
(vi) the self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial.

9. The method of claim 7 or 8, wherein:
the droplets are contacted with the gelling bath for a range of 10 minutes to 1 hour and the resulting microspheres degrade over a period of greater than about 20 minutes to less than about 4 hours;
the droplets are contacted with the gelling bath for a range of 1 hour to 12 hours and the resulting microspheres degrade over a period of greater than about 5 days to less than about 30 days; or
the droplets are contacted with the gelling bath for a range of 12 hours to 24 hours and the resulting microspheres degrade over a period of greater than about 30 days.

10. The method of any one of claims 7-9, wherein at least one of (i)-(iv) applies:
(i) the residual water content of the microsphere is between about 1% by mass and about 10% by mass;
(ii) the dehydrating comprises lyophilizing the microsphere or drying the microsphere using super critical CO₂;
(iii) the sterilizing comprises irradiating the microsphere with 6-10 kGy of gamma radiation; or
(iv) the precursor solution and/or the gelling bath further comprise an anti-inflammatory agent, a chemotherapeutic agent, an antioxidant, or a combination thereof.

11. A method of preparing a photopolymerized microsphere capable of self-degradation upon rehydration, the method comprising:
forming droplets from a precursor solution, the precursor solution comprising:
(i) an enzyme pre-treated by varying temperature, by varying pH, and/or with a metal-ion enzyme inhibitor;
(ii) a biomaterial comprising a photo-crosslinkable moiety;
(iii) a photoinitiator;
irradiating the droplets, thereby cross-linking the biomaterial to form a photo-polymerized biologically derived microsphere encapsulating the enzyme; and
dehydrating, and optionally sterilizing, the microsphere thereby substantially removing water from the microsphere.

12. The method of claim 11, wherein at least one of (i)-(vi) applies:
(i) the photo-crosslinkable moiety is selected from an acrylate group, a methacrylate group, a vinyl group, and an allyl group;
(ii) the precursor solution further comprises one or more cryoprotectants;
(iii) the enzyme is an enzyme that acts on the biomaterial, degrading the biomaterial;
(iv) the biomaterial comprises a polysaccharide, a protein, or a glycoprotein;
(v) the microsphere self-degrades in less than about 4 hours, over a period of greater than about 2 days to less than about 5 days, over a period of greater than about 5 days to less than about 30 days, or greater than about 30 days; or
(vi) the self-degradation of the microsphere is controlled by one or more of: the pre-treatment of the enzyme, the concentration of the enzyme in the microsphere, the enzyme activity, the predetermined molecular weight of the biomaterial, and the amount of time that the droplets are irradiated.

13. The method of claim 11 or 12, wherein the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase.

14. The method of any one of claims 11-13, wherein at least one of (i)-(iv) applies:
(i) the residual water content of the microsphere is between about 1% by mass and about 10% by mass;
(ii) the dehydrating comprises lyophilizing the microsphere or drying the microsphere using super critical CO₂;
(iii) the sterilizing comprises irradiating the microsphere with 6-10 kGy of gamma radiation; or
(iv) the precursor solution further comprises an anti-inflammatory agent, a chemotherapeutic agent, an antioxidant, or a combination thereof.

15. A method of preparing a microsphere capable of self-degradation upon rehydration, the method comprising:
forming droplets from a precursor solution, the precursor solution comprising:
(i) a biomaterial comprising a covalently crosslinkable moiety; and
(ii) a homo-bifunctional crosslinking agent or a heterobifunctional crosslinking agent;
covalently cross-linking the biomaterial to form a biologically derived microsphere;
swelling an enzyme that has been pre-treated by varying temperature, by varying pH, and/or with a metal-ion enzyme inhibitor into the microsphere such that the biologically derived microsphere encapsulates the enzyme; and
dehydrating, and optionally sterilizing, the microsphere thereby substantially removing water from the microsphere.

16. The method of claim 15, wherein at least one of (i)-(vi) applies:
(i) the covalently crosslinkable moiety comprises an amine group or a carboxyl group;
(ii) the precursor solution further comprises one or more cryoprotectants;
(iii) the enzyme is an enzyme that acts on the biomaterial, degrading the biomaterial;
(iv) the biomaterial comprises a polysaccharide, a protein, or a glycoprotein;
(v) the microsphere self-degrades in less than about 4 hours, over a period of greater than about 2 days to less than about 5 days, over a period of greater than about 5 days to less than about 30 days, or greater than about 30 days; or
(vi) the self-degradation of the microsphere is controlled by one or more of: the pre-treatment of the enzyme, the concentration of the enzyme in the microsphere, the enzyme activity, the predetermined molecular weight of the biomaterial, and the homo-bifunctional crosslinking agent or heterobifunctional crosslinking used to crosslink the biomaterial.

17. The method of claim 15 or 16, wherein the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase.

18. The method of any one of claims 15-17, wherein at least one of (i)-(iv) applies:
(i) the residual water content of the microsphere is between about 1% by mass and about 10% by mass;
(ii) the dehydrating comprises lyophilizing the microsphere or drying the microsphere using super critical CO₂;
(iii) the sterilizing comprises irradiating the microsphere with 6-10 kGy of gamma radiation; or
(iv) the precursor solution further comprises an anti-inflammatory agent, a chemotherapeutic agent, an antioxidant, or a combination thereof.

19. A method of preparing a microsphere capable of self-degradation upon rehydration, the method comprising:
forming droplets from a precursor solution, the precursor solution comprising:
(i) a biomaterial comprising a covalently crosslinkable moiety;
(ii) an enzyme that has been pre-treated by varying temperature, by varying pH, and/or with a metal-ion enzyme inhibitor; and
(iii) a homo-bifunctional crosslinking agent or a heterobifunctional crosslinking agent;
covalently cross-linking the biomaterial to form a biologically derived microsphere encapsulating the enzyme; and
dehydrating, and optionally sterilizing, the microsphere thereby substantially removing water from the microsphere.

20. The method of claim 19, wherein at least one of (i)-(vi) applies:
(i) the covalently crosslinkable moiety comprises an amine group or a carboxyl group;
(ii) the precursor solution further comprises one or more cryoprotectants;
(iii) the enzyme is an enzyme that acts on the biomaterial, degrading the biomaterial;
(iv) the biomaterial comprises a polysaccharide, a protein, or a glycoprotein; or
(v) the microsphere self-degrades in less than about 4 hours, over a period of greater than about 2 days to less than about 5 days, over a period of greater than about 5 days to less than about 30 days, or greater than about 30 days; or
(vi) the self-degradation of the microsphere is controlled by one or more of: the pre-treatment of the enzyme, the concentration of the enzyme in the microsphere, the enzyme activity, the predetermined molecular weight of the biomaterial, and the homo-bifunctional crosslinking agent or heterobifunctional crosslinking used to crosslink the biomaterial.

21. The method of claim 19 or 20, wherein the microsphere comprises alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase.

22. The method of any one of claims 19-21, wherein at least one of (i)-(iv) applies:
(i) the residual water content of the microsphere is between about 1% by mass and about 10% by mass;
(ii) the dehydrating comprises lyophilizing the microsphere or drying the microsphere using super critical CO₂;
(iii) the sterilizing comprises irradiating the microsphere with 6-10 kGy of gamma radiation; or
(iv) the precursor solution further comprises an anti-inflammatory agent, a chemotherapeutic agent, an antioxidant, or a combination thereof.

23. A method of preparing a thermogelated microsphere capable of self-degradation upon rehydration, the method comprising:
heating a precursor solution comprising a biomaterial to melt the biomaterial;
adding to the precursor solution an enzyme pre-treated by varying temperature, by varying pH, and/or with a metal-ion enzyme inhibitor;
forming droplets from the precursor solution;
cooling the droplets to form a thermogelated biologically derived microsphere encapsulating the enzyme; and
dehydrating, and optionally sterilizing, the microsphere thereby substantially removing water from the microsphere.

24. The method of claim 23, wherein at least one of (i)-(v) applies:
(i) the precursor solution further comprises one or more cryoprotectants;
(ii) the enzyme is an enzyme that acts on the biomaterial, degrading the biomaterial;
(iii) the biomaterial comprises a polysaccharide, a protein, or a glycoprotein;
(iv) the microsphere self-degrades in less than about 4 hours, over a period of greater than about 2 days to less than about 5 days, over a period of greater than about 5 days to less than about 30 days, or greater than about 30 days; or
(v) the self-degradation of the microsphere is controlled by one or more of: the pre-treatment of the enzyme, the concentration of the enzyme in the microsphere, the enzyme activity, and the predetermined molecular weight of the biomaterial.

25. The method of claim 23 or 24, wherein the microsphere comprises pectin particles encapsulating pectinase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, or agar/agarose particles encapsulating agarose.

26. The method of any one of claims 23-25, wherein at least one of (i)-(iv) applies:
(i) the residual water content of the microsphere is between about 1% by mass and about 10% by mass;
(ii) the dehydrating comprises lyophilizing the microsphere or drying the microsphere using super critical CO₂;
(iii) the sterilizing comprises irradiating the microsphere with 6-10 kGy of gamma radiation; or
(iv) the precursor solution further comprises an anti-inflammatory agent, a chemotherapeutic agent, an antioxidant, or a combination thereof.

27. A method of preparing a microsphere capable of self-degradation, the method comprising:
forming a precursor solution, the precursor solution comprising:
(i) an enzyme; and
(ii) a biomaterial;
passing the precursor solution through a needle under the influence of an electrostatic potential, forming droplets; and
contacting the droplets with a gelling bath comprising a divalent metal ion, thereby cross-linking the biomaterial to form a biologically derived microsphere encapsulating the enzyme.

28. The method of claim 27, further comprising dehydrating, and optionally sterilizing, the microsphere thereby substantially removing water from the microsphere to form a microsphere capable of self-degradation upon rehydration.

29. The method of claim 27 or 28, wherein at least one of (i)-(v) applies:
(i) the precursor solution further comprises one or more cryoprotectants;
(ii) the enzyme is an enzyme that acts on the biomaterial, degrading the biomaterial;
(iii) the biomaterial comprises a polysaccharide, a protein, or a glycoprotein;
(iv) the microsphere self-degrades in less than about 4 hours, over a period of greater than about 2 days to less than about 5 days, over a period of greater than about 5 days to less than about 30 days, or greater than about 30 days; or
(v) the self-degradation of the microsphere is controlled by one or more of: the pre-treatment of the enzyme, the concentration of the enzyme in the microsphere, the enzyme activity, the predetermined molecular weight of the biomaterial, the divalent metal ion used to crosslink the biomaterial, and the amount of divalent metal ion used to crosslink the biomaterial.

30. The method of any one of claims 27-29, wherein the microsphere comprises: alginate particles encapsulating alginate lyase, pectin particles encapsulating pectinase, hyaluronic acid particles encapsulating hyaluronidase, gelatin particles encapsulating a matrix metalloproteinase or protease, albumin particles encapsulating peptidase, collagen particles encapsulating protease, fibrinogen particles encapsulating plasmin, silk fibrin particles encapsulating protease, starch particles encapsulating amylase, chitosan particles encapsulating chitosanase or lysozyme, agar/agarose particles encapsulating agarase, carrageenan particles encapsulating carrageenase, pullulan particles encapsulating pullulanase, dextran particles encapsulating dextranase, b-glycan particles encapsulating b-glycanase, cellulose particles encapsulating cellulase, or lignin particles encapsulating ligninase.

31. The method of any one of claims 28-30, wherein at least one of (i)-(iv) applies:
(i) the residual water content of the microsphere is between about 1% by mass and about 10% by mass;
(ii) the dehydrating comprises lyophilizing the microsphere or drying the microsphere using super critical CO₂;
(iii) the sterilizing comprises irradiating the microsphere with 6-10 kGy of gamma radiation; or
(iv) the precursor solution and/or the gelling bath further comprise an anti-inflammatory agent, a chemotherapeutic agent, an antioxidant, or a combination thereof.

32. A method of inducing a self-degrading embolism in a subject in need thereof, comprising administering a plurality of the microspheres of any one of claims 1-6 into a blood vessel of the subject.

33. The method of claim 32, wherein the blood vessel is a geniculate artery and/or the method induces a prostate arterial embolism, induces a uterine artery embolism, or the microsphere comprises a chemotherapeutic agent or is mixed with a chemotherapeutic agent and the method induces a transarterial chemoembolism (TACE).

34. A method of treating a disease or disorder in a subject in need thereof, comprising administering to the subject a plurality of the microspheres of any one of claims 1-6.

35. The method of claim 34, wherein the disease or disorder is selected from tendinopathy, osteoarthritis, frozen shoulder, tennis elbow (lateral epicondylitis), golfer's elbow (medial epicondylitis), pitcher's elbow (flexor tendinitis), Achilles tendinopathy, plantar fasciitis, symptomatic accessory navicular bone, hamstring tendinopathy, jumper's knee (patellar tendonitis), runner's knee (patellofemoral pain syndrome (PFPS)), pes anserine bursitis (knee pain), posterior tibial muscle tendinopathy, wrist (TFCC—Triangular FibroCartilage Complex) tendinopathy, trigger finger (stenosing flexor tenosynovitis), and haemarthrosis.

36. A method of rapidly degrading a microsphere in a subject, comprising administering to the subject a bail out solution, wherein a plurality of microspheres of any one of claims 1-6 was previously administered to the subject and the bail out solution comprises an enzyme capable of degrading the microspheres.

37. The method of claim 36, wherein the enzyme is complementary to the biomaterial used to form the plurality of microspheres.

38. The method of claim 36 or 37, wherein:
the microsphere comprises alginate particles encapsulating alginate lyase and the enzyme is alginate lyase,
the microsphere comprises pectin particles encapsulating pectinase and the enzyme is pectinase,
the microsphere comprises hyaluronic acid particles encapsulating hyaluronidase and the enzyme is hyaluronidase,
the microsphere comprises gelatin particles encapsulating a matrix metalloproteinase or protease and the enzyme is a matrix metalloproteinase or protease,
the microsphere comprises albumin particles encapsulating peptidase and the enzyme is peptidase,
the microsphere comprises collagen particles encapsulating protease and the enzyme is protease,
the microsphere comprises fibrinogen particles encapsulating plasmin and the enzyme is plasmin,
the microsphere comprises silk fibrin particles encapsulating protease and the enzyme is protease,
the microsphere comprises starch particles encapsulating amylase and the enzyme is amylase,
the microsphere comprises chitosan particles encapsulating chitosanase or lysozyme and the enzyme is chitosanase or lysozyme,
the microsphere comprises agar/agarose particles encapsulating agarase and the enzyme is agarase,
the microsphere comprises carrageenan particles encapsulating carrageenase and the enzyme is carrageenase,
the microsphere comprises pullulan particles encapsulating pullulanase and the enzyme is pullulanase,
the microsphere comprises dextran particles encapsulating dextranase and the enzyme is dextranase,
the microsphere comprises b-glycan particles encapsulating b-glycanase and the enzyme is b-glycanase,
the microsphere comprises cellulose particles encapsulating cellulase and the enzyme is cellulase, or
the microsphere comprises lignin particles encapsulating ligninase and the enzyme is ligninase.

39. The method of any one of claims 36-38, wherein the bail out solution further comprises a divalent metal chelator.

40. A method of rapidly degrading a divalent metal ion crosslinked microsphere in a subject, comprising administering to the subject a bail out solution, wherein a plurality of divalent metal ion crosslinked microspheres of any one of claims 1-6 was previously administered to the subject and the bail out solution comprises an anion, a phosphate buffer, or a combination thereof.

41. The method of claim 40, wherein at least one of (i)-(iii) applies:
(i) the anion comprises citrate;
(ii) the phosphate buffer comprises phosphate buffered saline; and
(iii) the microsphere comprises alginate particles encapsulating alginate lyase, pectin particles encapsulating pectinase, or carrageenan particles encapsulating carrageenase.

42. The method of claim 40 or 41, wherein the microsphere comprises alginate particles encapsulating alginate lyase, pectin particles encapsulating pectinase, or carrageenan particles encapsulating carrageenase; and wherein the anion comprises citrate; or
wherein the microsphere comprises alginate particles encapsulating alginate lyase, pectin particles encapsulating pectinase, or carrageenan particles encapsulating carrageenase; and wherein the phosphate buffer comprises phosphate buffered saline.

43. A kit comprising:
(i)
a plurality of microspheres of any one of claims 1-6; and
an enzyme capable of rapidly degrading the microspheres when dissolved to form a solution; or
(ii)
a plurality of divalent metal ion crosslinked microspheres of any one of claims 1-6; and
an inorganic salt capable of rapidly degrading the microspheres when dissolved to form a solution.

44. The kit of claim 43, wherein the enzyme of (i) is complementary to the biomaterial used to form the plurality of microspheres.

45. The kit of claim 43 or 44, wherein, for the plurality of microspheres of (i):
the microspheres comprise alginate particles encapsulating alginate lyase and the enzyme is alginate lyase,
the microspheres comprise pectin particles encapsulating pectinase and the enzyme is pectinase,
the microspheres comprise hyaluronic acid particles encapsulating hyaluronidase and the enzyme is hyaluronidase,
the microspheres comprise gelatin particles encapsulating a matrix metalloproteinase or protease and the enzyme is a matrix metalloproteinase or protease,
the microspheres comprise albumin particles encapsulating peptidase and the enzyme is peptidase,
the microspheres comprise collagen particles encapsulating protease and the enzyme is protease,
the microspheres comprise fibrinogen particles encapsulating plasmin and the enzyme is plasmin,
the microspheres comprise silk fibrin particles encapsulating protease and the enzyme is protease,
the microspheres comprise starch particles encapsulating amylase and the enzyme is amylase,
the microspheres comprise chitosan particles encapsulating chitosanase or lysozyme and the enzyme is chitosanase or lysozyme,
the microspheres comprise agar/agarose particles encapsulating agarase and the enzyme is agarase,
the microspheres comprise carrageenan particles encapsulating carrageenase and the enzyme is carrageenase,
the microspheres comprise pullulan particles encapsulating pullulanase and the enzyme is pullulanase,
the microsphere comprises dextran particles encapsulating dextranase and the enzyme is dextranase,
the microspheres comprise b-glycan particles encapsulating b-glycanase and the enzyme is b-glycanase,
the microspheres comprise cellulose particles encapsulating cellulase and the enzyme is cellulase, or
the microspheres comprise lignin particles encapsulating ligninase and the enzyme is ligninase.

46. The kit of claim 43, wherein, for the plurality of microspheres of (ii): the microspheres comprise alginate particles encapsulating alginate lyase, pectin particles encapsulating pectinase, or carrageenan particles encapsulating carrageenase.

47. The kit of claim 43 or 46, wherein the inorganic salt of (ii) releases citrate or phosphate when dissolved to form a solution.
